(19) United States
(12) Patent Application Publication
(10) Pub. No.: US 2024/0218391 A1
(43) Pub. Date: Jul. 4, 2024

(54) VESTIBULAR SUPPORTING CELL PROMOTERS AND USES THEREOF

(71) Applicant: Decibel Therapeutics, Inc., Boston, MA (US)

(72) Inventors: Joseph BURNS, Newton, MA (US); Tyler GIBSON, Boston, MA (US); Gabriela PREGERNIG, Cambridge, MA (US); Kathy SO, Brighton, MA (US)

(21) Appl. No.: 18/289,031

(22) PCT Filed: May 4, 2022
(86) PCT No.: PCT/US2022/027679

§ 371 Date: Oct. 31, 2023

Related U.S. Application Data
(60) Provisional application No. 63/184,015, filed on May 4, 2021.

(51) Int. Cl. C12N 15/86; A61K 48/00; C07K 14/47

(52) U.S. Cl. CPC C12N 15/86; A61K 48/0008; C07K 14/4705; C12N 2750/14143; C12N 2830/008

(57) ABSTRACT

The disclosure provides polynucleotides containing SLC6A14 promoters, as well as vectors containing the same, that can be used to promote expression of a transgene in vestibular supporting cells. The polynucleotides described herein may be operably linked to a transgene, such as a transgene encoding a protein of interest, so as to promote vestibular supporting cell expression of the transgene. The polynucleotides described herein may be operably linked to a transgene and used for the treatment of subjects having or at risk of developing vestibular dysfunction.

### SEQUENCE LISTING

The instant application contains a Sequence Listing which has been submitted electronically in ASCII format and is hereby incorporated by reference in its entirety. The ASCII copy, created on Apr. 28, 2022, is named 51471-010WO2_Sequence_Listing_4_28_22_ST25 and is 41,804 bytes in size.

### BACKGROUND

Vestibular dysfunction is a major public health issue that has profound consequences on quality of life. Approximately 35% of US adults age 40 years and older exhibit balance disorders and this proportion dramatically increases with age, leading to disruption of daily activities, decline in mood and cognition, and an increased prevalence of falls among the elderly. Vestibular dysfunction is often acquired, and has a variety of causes, including disease or infection, head trauma, ototoxic drugs, and aging. A common factor in the etiology of vestibular dysfunction is the damage to vestibular hair cells of the inner ear. Thus, therapies aimed at restoring hair cell function would be beneficial to patients suffering from vestibular dysfunction. Vestibular supporting cells are known to spontaneously differentiate into hair cells following damage and may, therefore, serve as a suitable therapeutic target for restoring hair cell function.

### SUMMARY OF THE INVENTION

The invention provides compositions and methods for promoting the expression of a gene of interest, such as a gene that promotes or improves hair cell or supporting cell function, regeneration, maturation, proliferation, or survival, in specific cell types. The compositions and methods described herein relate to Solute Carrier Family 6 Member 14 (SLC6A14) promoter sequences that can be used to induce expression of a transgene in vestibular supporting cells (VSCs) of the inner ear. The SLC6A14 promoter sequences described herein may be operably linked to a transgene and may be administered to a patient to treat vestibular dysfunction (e.g., vertigo, dizziness, imbalance, bilateral vestibulopathy, bilateral vestibular hypofunction, oscillopsia, or a balance disorder). The SLC6A14 promoter sequences described herein demonstrate high cell type-specificity by driving high expression of an operably linked transgene in vestibular supporting cells with much lower expression in other inner ear cell types, such as hair cells.

In a first aspect, the invention provides a nucleic acid vector comprising a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1. In some embodiments, the polynucleotide has at least 90% sequence to SEQ ID NO: 1. In some embodiments, the polynucleotide has at least 95% sequence to SEQ ID NO: 1. In some embodiments, the polynucleotide has the sequence of SEQ ID NO: 1.

In some embodiments, the polynucleotide is operably linked to a transgene. In some embodiments, the transgene is a heterologous transgene. In some embodiments, the transgene encodes a protein (e.g., a therapeutic protein or a reporter protein), a short hairpin RNA (shRNA), an antisense oligonucleotide (ASO), a nuclease (e.g., CRISPR Associated Protein 9 (Cas9), Transcription Activator-Like Effector Nuclease (TALEN), Zinc Finger Nuclease (ZFN), or guide RNA (gRNA)), or a microRNA. In some embodiments, the transgene encodes a protein.

In some embodiments, the polynucleotide directs vestibular supporting cell (VSC)-specific expression of the protein (e.g., a therapeutic protein or a reporter protein), shRNA, ASO, nuclease, or microRNA in a mammalian VSC. In some embodiments, the VSC is a human VSC.

In some embodiments, the protein encoded by the transgene operably linked to the polynucleotide is Spalt Like Transcription Factor 2 (Sall2), Calmodulin Binding Transcription Activator 1 (Camta1), Hes Related Family BHLH Transcription Factor With YRPW Motif 2 (Hey2), Gata Binding Protein 2 (Gata2), Hes Related Family BHLH Transcription Factor With YRPW Motif 1 (Hey1), Ceramide Synthase 2 (Lass2), SRY-Box 10 (Sox10), GATA Binding Protein 3 (Gata3), Cut Like Homeobox 1 (Cux1), Nuclear Receptor Subfamily 2 Group F Member (Nr2f1), Hes Related Family BHLH Transcription Factor (Hes1), RAR Related Orphan Receptor B (Rorb), Jun Proto-Oncogene AP-1 Transcription Factor Subunit (Jun), Zinc Finger Protein 667 (Zfp667), LIM Homeobox 3 (Lhx3), Nescient Helix-Loop-Helix 1 (Nhlh1), MAX Dimerization Protein 4 (Mxd4), Zinc Finger MIZ-Type Containing 1 (Zmiz1), Myelin Transcription Factor 1 (Myt1), Signal Transducer And Activator Of Transcription 3 (Stat3), BarH Like Homeobox 1 (Barhl1), Thymocyte Selection Associated High Mobility Group Box (Tox), Prospero Homeobox 1 (Prox1), Nuclear Factor I A (Nfia), Thyroid Hormone Receptor Beta (Thrb), MYCL Proto-Oncogene BHLH Transcription Factor (Mycl1), Lysine Demethylase 5A (Kdm5a), CAMP Responsive Element Binding Protein 3 Like 4 (Creb314), ETS Variant 1 (Etv1), Paternally Expressed 3 (Peg3), BTB Domain And CNC Homolog 2 (Bach2), ISL LIM Homeobox 1 (Isl1), Zinc Finger And BTB Domain Containing 38 (Zbtb38), Limb Bud And Heart Development (Lbh), Tubby Bipartite Transcription Factor (Tub), Ubiquitin C (Hmg20), RE1 Silencing Transcription Factor (Rest), Zinc Finger Protein 827 (Zfp827), AF4/FMR2 Family Member 3 (Aff3), PBX/Knotted 1 Homeobox 2 (Pknox2), AT-Rich Interaction Domain 3B (Arid3b), MLX Interacting Protein (MIxip), Zinc Finger Protein (Zfp532), IKAROS Family Zinc Finger 2 (Ikzf2), Spalt Like Transcription Factor 1 (Sall1), SIX Homeobox 2 (Six2), Spalt Like Transcription Factor 3 (Sall3), Lin-28 Homolog B (Lin28b), Regulatory Factor X7 (Rfx7), Brain Derived Neurotrophic Factor (Bdnf), Growth Factor Independent 1 Transcriptional Repressor (Gfi1), POU Class 4 Homeobox 3 (Pou4f3), MYC Proto-Oncogene BHLH Transcription Factor (Myc), β-catenin (Ctnnb1), SRY-Box 2 (Sox2), SRY-Box 4 (Sox4), SRY-Box 11 (Sox11), TEA Domain Transcription Factor 2 (Tead2), Atonal BHLH Transcription Factor 1 (Atoh1), or an Atoh1 variant.

In some embodiments, the protein encoded by the transgene operably linked to the polynucleotide is Atoh1 or an Atoh1 variant. In some embodiments, the Atoh1 variant has one or more amino acid substitutions selected from the group consisting of S328A, S331A, S334A, S328A/S331A, S328A/S334A, S331A/S334A, and S328A/S331A/S334 relative to SEQ ID NO: 4. In some embodiments, the protein is Atoh1 (e.g., human Atoh1). In some embodiments, the Atoh1 protein comprises the sequence of SEQ ID NO: 4 or a variant thereof having one or more (e.g., 1, 2, 3, 4, 5, 6, 7, 8, 9, 10, 11, 12, 13, 14, 15, 16, 17, 18, 19, 20 or more) conservative amino acid substitutions. In some embodiments, the Atoh1 protein comprises the sequence of SEQ ID NO: 6 or a variant thereof having one or more (e.g., 1, 2, 3, 4, 5, 6, 7, 8, 9, 10, 11, 12, 13, 14, 15, 16, 17, 18, 19, 20 or more) conservative amino acid substitutions. In some embodiments, no more than 10% (10%, 9%, 8%, 7%, 6%, 5%, 4%, 3%, 2%, 1%, or fewer) of the amino acids in the Atoh1 protein variant are conservative amino acid substitutions. In some embodiments, the Atoh1 protein has the sequence of SEQ ID NO: 4. In some embodiments, the Atoh1 protein is encoded by the sequence of SEQ ID NO: 5. In some embodiments, the Atoh1 protein has the sequence of SEQ ID NO: 6. In some embodiments, the Atoh1 protein is encoded by the sequence of SEQ ID NO: 7.

In some embodiments, the nucleic acid vector further includes inverted terminal repeat sequences (ITRs). In embodiments in which the nucleic acid vector includes a polynucleotide of the invention operably linked to a transgene, the nucleic acid vector includes a first ITR sequence 5′ of the polynucleotide and a second ITR sequence 3′ of the transgene. In some embodiments, the ITRs are AAV2 ITRs. In some embodiments, the ITRs have at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to AAV2 ITRs.

In some embodiments, the nucleic acid vector further includes a polyadenylation (poly(A)) sequence. In some embodiments, the poly(A) sequence is a bovine growth hormone (bGH) poly(A) signal sequence. In embodiments in which the nucleic acid vector includes a polynucleotide of the invention operably linked to a transgene, the poly(A) sequence is positioned 3′ of the transgene. In embodiments in which the nucleic acid vector includes first and second ITR sequences and a polynucleotide of the invention operably linked to a transgene, the poly(A) sequence is positioned 3′ of the transgene and 5′ of the second ITR sequence.

In some embodiments, the nucleic acid vector further includes a Woodchuck Posttranscriptional Regulatory Element (WPRE). In some embodiments, the WPRE has the sequence of SEQ ID NO: 8 or SEQ ID NO: 9. In embodiments in which the nucleic acid vector includes a polynucleotide of the invention operably linked to a transgene, the WPRE is positioned 3′ of the transgene. In embodiments in which the nucleic acid vector includes a polynucleotide of the invention operably linked to a transgene and a poly(A) sequence, the WPRE is positioned 3′ of the transgene and 5′ of the poly(A) sequence.

In some embodiments, the nucleic acid vector contains a polynucleotide sequence comprising the sequence of nucleotides 219-3831 of SEQ ID NO: 10.

In some embodiments, the nucleic acid vector contains a polynucleotide sequence comprising the sequence of nucleotides 219-3822 of SEQ ID NO: 11.

In some embodiments, the nucleic acid vector of the invention includes an SLC6A14 promoter (e.g., the polynucleotide of SEQ ID NO: 1) operably linked to a polynucleotide sequence encoding human Atoh1 (human ATOH1 protein=RefSeq Accession No. NP_005163 (SEQ ID NO: 4); mRNA sequence=RefSeq Accession No. NM_005172). In some more specific embodiments, the nucleic acid vector of the invention includes an SLC6A14 promoter of SEQ ID NO: 1 operably linked to a polynucleotide sequence encoding human Atoh1 (e.g., a polynucleotide sequence encoding SEQ ID NO: 4, such as the polynucleotide sequence of SEQ ID NO: 5). In some even more specific embodiments, the nucleic acid vector includes, in 5′ to 3′ order, a first inverted terminal repeat; an SLC6A14 promoter of SEQ ID NO: 1; a polynucleotide sequence encoding human Atoh1 operably linked to the SLC6A14 promoter; a polyadenylation sequence; and a second inverted terminal repeat. In further, more specific embodiments, the nucleic acid vector includes, in 5′ to 3′ order, a first inverted terminal repeat; an SLC6A14 promoter of SEQ ID NO: 1; a polynucleotide sequence encoding human Atoh1 operably linked to the SLC6A14 promoter; a WPRE; a polyadenylation sequence; and a second inverted terminal repeat. In even more specific embodiments, the nucleic acid vector includes nucleotides 219-3831 of SEQ ID NO: 10, flanked by inverted terminal repeats. In even more specific embodiments, the nucleic acid vector includes nucleotides 219-3831 of SEQ ID NO: 10, flanked by inverted terminal repeats, in which the 5′ inverted terminal repeat has at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to nucleotides 1-130 of SEQ ID NO: 10; and in which the 3′ inverted terminal repeat has at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to nucleotides 3919-4048 of SEQ ID NO: 10.

In some embodiments, the nucleic acid vector of the invention includes an SLC6A14 promoter (e.g., the polynucleotide of SEQ ID NO: 1) operably linked to a polynucleotide sequence encoding murine Atoh1 (murine ATOH1 protein=UniProt P48985 (SEQ ID NO: 6); mRNA sequence=RefSeq Accession No. NM_007500.5). In some more specific embodiments, the nucleic acid vector of the invention includes an SLC6A14 promoter of SEQ ID NO: 1 operably linked to a polynucleotide sequence encoding murine Atoh1 (e.g., a polynucleotide sequence encoding SEQ ID NO: 6, such as the polynucleotide sequence of SEQ ID NO: 7). In some even more specific embodiments, the nucleic acid vector includes, in 5′ to 3′ order, a first inverted terminal repeat; an SLC6A14 promoter of SEQ ID NO: 1; a polynucleotide sequence encoding murine Atoh1 operably linked to the SLC6A14 promoter; a polyadenylation sequence; and a second inverted terminal repeat. In further, more specific embodiments, the nucleic acid vector includes, in 5′ to 3′ order, a first inverted terminal repeat; an SLC6A14 promoter of SEQ ID NO: 1; a polynucleotide sequence encoding murine Atoh1 operably linked to the SLC6A14 promoter; a WPRE; a polyadenylation sequence; and a second inverted terminal repeat. In even more specific embodiments, the nucleic acid vector includes nucleotides 219-3822 of SEQ ID NO: 11, flanked by inverted terminal repeats. In even more specific embodiments, the nucleic acid vector includes nucleotides 219-3822 of SEQ ID NO: 11, flanked by inverted terminal repeats, in which the 5′ inverted terminal repeat has at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to nucleotides 1-130 of SEQ ID NO: 11; and in which the 3′ inverted terminal repeat has at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to nucleotides 3910-4039 of SEQ ID NO: 11.

In some embodiments, the nucleic acid vector is a viral vector, plasmid, cosmid, or artificial chromosome. In some embodiments, the nucleic acid vector is a viral vector selected from the group including an adeno-associated virus (AAV), an adenovirus, and a lentivirus. In some embodiments, the viral vector is an AAV vector. In some embodiments, the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S capsid. In some embodiments, the AAV vector has an AAV1 capsid. In some embodiments, the AAV vector has an AAV9 capsid. In some embodiments, the AAV vector has an AAV6 capsid. In some embodiments, the AAV vector has an AAV8 capsid. In some embodiments, the AAV vector has an Anc80 capsid. In some embodiments, the AAV vector has an Anc80L65 capsid. In some embodiments, the AAV vector has a DJ/9 capsid. In some embodiments, the AAV vector has a 7m8 capsid. In some embodiments, the AAV vector has an AAV2 capsid. In some embodiments, the AAV vector has a PHP.B capsid. In some embodiments, the AAV vector has an AAV2quad(Y-F) capsid. In some embodiments, the AAV vector has a PHP.S capsid. In some embodiments, the AAV vector has a PHP.eB capsid. In some embodiments, the AAV vector has an AAV3 capsid. In some embodiments, the AAV vector has an AAV4 capsid. In some embodiments, the AAV vector has an AAV5 capsid. In some embodiments, the AAV vector has an AAV7 capsid.

It should be understood by those of ordinary skill in the art that the creation of a viral vector of the invention typically requires the use of a plasmid of the invention together with additional plasmids that provide required elements for proper viral packaging and viability (e.g., for AAV, plasmids providing the appropriate AAV rep gene, cap gene and other genes (e.g., E2A and E4)). The combination of those plasmids in a producer cell line produces the viral vector. However, it will be understood by those of skill in the art, that for any given pair of inverted terminal repeat sequences in a transfer plasmid of the invention that is used to create the viral vector, the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom.

In another aspect, the invention provides a composition containing a nucleic acid vector of the invention. In some embodiments, the composition further includes a pharmaceutically acceptable carrier, diluent, or excipient.

In another aspect, the invention provides a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 operably linked to a transgene. In some embodiments, the polynucleotide has the sequence of SEQ ID NO: 1.

In some embodiments, the transgene is a heterologous transgene. In some embodiments of the foregoing aspect, the transgene encodes a protein (e.g., a therapeutic protein or a reporter protein), an shRNA, an ASO, a nuclease (e.g., Cas9, TALEN, ZFN, or gRNA), or a microRNA. In some embodiments, the transgene encodes a protein.

In some embodiments, the protein is Sox9, Sall2, Camta1, Hey2, Gata2, Hey1, Lass2, Sox10, Gata3, Cux1, Nr2f1, Hes1, Rorb, Jun, Zfp667, Lhx3, Nhlh1, Mxd4, Zmiz1, Myt1, Stat3, Barhl1, Tox, Prox1, Nfia, Thrb, Mycl1, Kdm5a, Creb314, Etv1, Peg3, Bach2, Isl1, Zbtb38, Lbh, Tub, Hmg20, Rest, Zfp827, Aff3, Pknox2, Arid3b, Mixip, Zfp532, Ikzf2, Sall1, Six2, Sall3, Lin28b, Rfx7, Bdnf, Gfi1, Pou4f3, Myc, Ctnnb1, Sox2, Sox4, Sox11, Tead2, Atoh1, or an Atoh1 variant (e.g., an Atoh1 variant having one or more amino acid substitutions selected from the group consisting of S328A, S331A, S334A, S328A/S331A, S328A/S334A, S331A/S334A, and S328A/S331A/S334). In some embodiments, the protein is Atoh1 (e.g., human Atoh1). In some embodiments, the Atoh1 protein comprises the sequence of SEQ ID NO: 4 or a variant thereof having one or more (e.g., 1, 2, 3, 4, 5, 6, 7, 8, 9, 10, 11, 12, 13, 14, 15, 16, 17, 18, 19, 20 or more) conservative amino acid substitutions. In some embodiments, no more than 10% (10%, 9%, 8%, 7%, 6%, 5%, 4%, 3%, 2%, 1%, or fewer) of the amino acids in the Atoh1 protein variant are conservative amino acid substitutions. In some embodiments, the Atoh1 protein has the sequence of SEQ ID NO: 4. In some embodiments, the Atoh1 protein is encoded by the sequence of SEQ ID NO: 5.

In another aspect, the invention provides a cell (e.g., a mammalian cell, e.g., a human cell, such as a VSC) including the polynucleotide or the nucleic acid vector of any of the foregoing aspects and embodiments. In some embodiments, the cell is a mammalian VSC. In some embodiments, the mammalian VSC is a human VSC. In some embodiments, the polynucleotide has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1.

In another aspect, the invention provides a method of expressing a transgene in a mammalian VSC by contacting the mammalian VSC with the nucleic acid vector or composition of any of the foregoing aspects and embodiments. In some embodiments, the transgene is specifically expressed in VSCs (e.g., expressed in a percentage of VSCs that is at least 2-fold, 5-fold, 10-fold, 50-fold, 100-fold, 200-fold, 300-fold, 400-fold, 500-fold or greater than the percentage of one or more other inner ear cells (e.g., hair cells) in which expression is observed). In some embodiments, the mammalian VSC is a human VSC.

In another aspect, the invention provides a method of treating a subject having or at risk of developing vestibular dysfunction by administering to an inner ear of the subject an effective amount of the nucleic acid vector or composition of any of the foregoing aspects and embodiments. In some embodiments, the vestibular dysfunction is vertigo, dizziness, imbalance, bilateral vestibulopathy (also known as bilateral vestibular hypofunction), oscillopsia, or a balance disorder. In some embodiments, the vestibular dysfunction is age-related vestibular dysfunction, head trauma-related vestibular dysfunction, disease or infection-related vestibular dysfunction, or ototoxic drug-induced vestibular dysfunction. In some embodiments, the vestibular dysfunction is associated with a genetic mutation. In some embodiments, the vestibular dysfunction is idiopathic vestibular dysfunction.

In another aspect, the invention provides a method of inducing or increasing vestibular hair cell regeneration in a subject in need thereof by administering to an inner ear of the subject an effective amount of the nucleic acid vector or composition of any of the foregoing aspects and embodiments.

In another aspect, the invention provides a method of inducing or increasing VSC proliferation in a subject in need thereof by administering to an inner ear of the subject an effective amount of the nucleic acid vector or composition of any of the foregoing aspects and embodiments.

In another aspect, the invention provides a method of inducing or increasing vestibular hair cell proliferation in a subject in need thereof by administering to an inner ear of the subject an effective amount of the nucleic acid vector or composition of any of the foregoing aspects and embodiments.

In another aspect, the invention provides a method of inducing or increasing vestibular hair cell maturation in a subject in need thereof by administering to an inner ear of the subject an effective amount of the nucleic acid vector or composition of any of the foregoing aspects and embodiments. In some embodiments, the vestibular hair cell is a regenerated vestibular hair cell.

In another aspect, the invention provides a method of increasing VSC survival in a subject in need thereof by administering to an inner ear of the subject an effective amount of the nucleic acid vector or composition of any of the foregoing aspects and embodiments.

In another aspect, the invention provides a method of increasing vestibular hair cell survival in a subject in need thereof by administering to an inner ear of the subject an effective amount of the nucleic acid vector or composition of any of the foregoing aspects and embodiments.

In another aspect, the invention provides a method of inducing or increasing vestibular hair cell innervation in a subject in need thereof by administering to an inner ear of the subject an effective amount of the nucleic acid vector or composition of any of the foregoing aspects and embodiments.

In some embodiments of any of the foregoing aspects, the subject has or is at risk of developing vestibular dysfunction (e.g., dizziness, vertigo, imbalance, bilateral vestibulopathy (bilateral vestibular hypofunction), oscillopsia, or a balance disorder).

In another aspect, the invention provides a method of treating a subject having or at risk of developing bilateral vestibulopathy by administering to an inner ear of the subject an effective amount of the nucleic acid vector or composition of any of the foregoing aspects and embodiments. In some embodiments, the bilateral vestibulopathy is ototoxic drug-induced bilateral vestibulopathy.

In some embodiments of any of the foregoing aspects, the ototoxic drug is selected from the group consisting of aminoglycosides, antineoplastic drugs, ethacrynic acid, furosemide, salicylates, and quinine.

In another aspect, the invention provides a method of treating a subject having or at risk of developing oscillopsia by administering to an inner ear of the subject an effective amount of the nucleic acid vector or composition of any of the foregoing aspects and embodiments.

In another aspect, the invention provides a method of treating a subject having or at risk of developing a balance disorder (e.g., imbalance) by administering to an inner ear of the subject an effective amount of the nucleic acid vector or composition of any of the foregoing aspects and embodiments.

In some embodiments of any of the foregoing aspects, the method further includes evaluating the vestibular function of the subject prior to administering the nucleic acid vector or composition. In some embodiments, the method further includes evaluating the vestibular function of the subject after administering the nucleic acid vector or composition.

In some embodiments of any of the foregoing aspects, the nucleic acid vector or composition is locally administered. In some embodiments, the nucleic acid vector or composition is administered to a semicircular canal. In some embodiments, the nucleic acid vector or composition is administered transtympanically or intratympanically (e.g., via transtympanic or intratympanic injection). In some embodiments, the nucleic acid vector or composition is administered to the perilymph or endolymph, such as through the oval window, round window, or semicircular canal (e.g., the horizontal canal), e.g., administration to a vestibular supporting cell. In some embodiments, the nucleic acid vector or composition of the invention is administered into the perilymph. In some embodiments, the nucleic acid vector or composition of the invention is administered into the endolymph. In some embodiments, the nucleic acid vector or composition of the invention is administered to or through the oval window. In some embodiments, the nucleic acid vector or composition of the invention is administered to or through the round window.

In some embodiments of any of the foregoing aspects, the nucleic acid vector or composition is administered in an amount sufficient to prevent or reduce vestibular dysfunction, delay the development of vestibular dysfunction, slow the progression of vestibular dysfunction, improve vestibular function, increase vestibular hair cell numbers, increase vestibular hair cell maturation, increase vestibular hair cell proliferation, increase vestibular hair cell regeneration, increase vestibular hair cell innervation, increase VSC proliferation, or increase VSC numbers.

In some embodiments of any of the foregoing aspects, the subject is a human.

In another aspect, the invention provides a kit containing a nucleic acid vector of the invention or a composition of the invention.

### Definitions

As used herein, “administration” refers to providing or giving a subject a therapeutic agent (e.g., a nucleic acid vector containing a Solute Carrier Family 6 Member 14 (SLC6A14) promoter operably linked to a transgene), by any effective route. Exemplary routes of administration are described herein below.

As used herein, the phrase “administering to the inner ear” refers to providing or giving a therapeutic agent described herein to a subject by any route that allows for transduction of inner ear cells. Exemplary routes of administration to the inner ear include administration into the perilymph or endolymph, such as to or through the oval window, round window, or semicircular canal (e.g., horizontal canal), or by transtympanic or intratympanic injection, e.g., administration to the vestibule, e.g., to a vestibular supporting cell.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally occurring amino acids in table 1 below.

TABLE 1
Representative physicochemical properties
of naturally occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non-polar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L, and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the terms “effective amount,” “therapeutically effective amount,” and a “sufficient amount” of a composition, vector construct, or viral vector described herein refer to a quantity sufficient to, when administered to the subject, including a mammal, for example a human, effect beneficial or desired results, including clinical results, and, as such, an “effective amount” or synonym thereto depends upon the context in which it is being applied. For example, in the context of treating vestibular dysfunction, it is an amount of the composition, vector construct, or viral vector sufficient to achieve a treatment response as compared to the response obtained without administration of the composition, vector construct, or viral vector. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, vector construct, or viral vector of the present disclosure is an amount which results in a beneficial or desired result in a subject as compared to a control. As defined herein, a therapeutically effective amount of a composition, vector construct, or viral vector of the present disclosure may be readily determined by one of ordinary skill by routine methods known in the art. Dosage regimen may be adjusted to provide the optimum therapeutic response.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human vestibular supporting cell).

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell, e.g., a human vestibular supporting cell). Exogenous materials include those that are provided from an external source to an organism or to cultured matter extracted there from.

As used herein, the term “exon” refers to a region within the coding region of a gene, the nucleotide sequence of which determines the amino acid sequence of the corresponding protein. The term exon also refers to the corresponding region of the RNA transcribed from a gene. Exons are transcribed into pre-mRNA and may be included in the mature mRNA depending on the alternative splicing of the gene. Exons that are included in the mature mRNA following processing are translated into protein, wherein the sequence of the exon determines the amino acid composition of the protein.

As used herein, the term “heterologous” refers to a combination of elements that is not naturally occurring. For example, a heterologous transgene refers to a transgene that is not naturally expressed by the promoter to which it is operably linked.

As used herein, the terms “increasing” and “decreasing” refer to modulating resulting in, respectively, greater or lesser amounts, of function, expression, or activity of a metric relative to a reference. For example, subsequent to administration of a composition in a method described herein, the amount of a marker of a metric (e.g., transgene expression) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, administration to the middle or inner ear, and administration to a mucous membrane of the subject, wherein the administration is intended to have a local and not a systemic effect.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “polynucleotide” refers to a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. The term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

As used herein, the term “promoter” refers to a recognition site on DNA that is bound by an RNA polymerase. The polymerase drives transcription of the transgene.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response, and other problem complications commensurate with a reasonable benefit/risk ratio.

As used herein, the term “transcription regulatory element” refers to a nucleic acid that controls, at least in part, the transcription of a gene of interest. Transcription regulatory elements may include promoters, enhancers, and other nucleic acids (e.g., polyadenylation signals) that control or help to control gene transcription. Examples of transcription regulatory elements are described, for example, in Lorence, Recombinant Gene Expression: Reviews and Protocols (Humana Press, New York, NY, 2012).

As used herein, the term “transfection” refers to any of a wide variety of techniques commonly used for the introduction of exogenous DNA into a prokaryotic or eukaryotic host cell, e.g., electroporation, lipofection, calcium phosphate precipitation, DEAE-dextran transfection, Nucleofection, squeeze-poration, sonoporation, optical transfection, magnetofection, impalefection and the like.

As used herein, the terms “subject” and “patient” refer to an animal (e.g., a mammal, such as a human). A subject to be treated according to the methods described herein may be one who has been diagnosed with vestibular dysfunction (e.g., dizziness, vertigo, or imbalance) or one at risk of developing these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transduction” and “transduce” refer to a method of introducing a vector construct or a part thereof into a cell. Wherein the vector construct is contained in a viral vector such as for example an AAV vector, transduction refers to viral infection of the cell and subsequent transfer and integration of the vector construct or part thereof into the cell genome.

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” includes a nucleic acid vector, e.g., a DNA vector, such as a plasmid, cosmid, or artificial chromosome, an RNA vector, a virus, or any other suitable replicon (e.g., viral vector). A variety of vectors have been developed for the delivery of polynucleotides encoding exogenous proteins into a prokaryotic or eukaryotic cell. Examples of such expression vectors are described in, e.g., Gellissen, Production of Recombinant Proteins: Novel Microbial and Eukaryotic Expression Systems (John Wiley & Sons, Marblehead, M A, 2006). Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of transgene as described herein include vectors that contain regulatory sequences, such as promoter and enhancer regions, which direct gene transcription. Other useful vectors for expression of a transgene contain polynucleotide sequences that enhance the rate of translation of the transgene or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the terms “vestibular supporting cell” and “VSC” refer to a collection of specialized epithelial cells in the vestibular system of the inner ear that are involved in vestibular hair cell development, survival, function, death, and phagocytosis. VSCs provide structural support to vestibular hair cells by anchoring them in the sensory epithelium and releasing neurotrophic factors important for hair cell innervation.

As used herein, the terms “vestibular supporting cell-specific expression” and “VSC-specific expression” refer to production of an RNA transcript or polypeptide primarily within vestibular supporting cells as compared to other cell types of the inner ear (e.g., vestibular hair cells, cochlear hair cells, cochlear supporting cells, glia, or other inner ear cell types). VSC expression of a transgene can be confirmed by comparing transgene expression (e.g., RNA or protein expression) between various cell types of the inner ear (e.g., VSCs vs. non-VSCs cells) using any standard technique (e.g., quantitative RT PCR, immunohistochemistry, western blot analysis, or measurement of the fluorescence of a reporter (e.g., GFP) operably linked to a promoter). A promoter that induces VSC-specific expression (a “VSC-specific promoter”) is a promoter that (i) induces expression (e.g., RNA or protein expression) of a transgene to which it is operably linked that is at least 50% greater (e.g., 50%, 75%, 100%, 125%, 150%, 175%, 200% greater or more) in VSCs, or (ii) induces expression of a transgene to which it is operably linked that is at least 2-fold greater (e.g. 5-fold, 10-fold, 50-fold, 100-fold, 200-fold, 300-fold, 400-fold, 500-fold greater or more) in VSCs, each as compared to at least 3 (e.g., 3, 4, 5, 6, 7, 8, 9, 10, or more) of the following inner ear cell types: vestibular ganglion cells, non-sensory epithelium cells of the vestibular organs, dark cells of the vestibular organs, mesenchymal cells of the vestibular organs, spiral ganglion cells, border cells, inner phalangeal cells, inner pillar cells, outer pillar cells, first row Deiter cells, second row Deiter cells, third row Deiter cells, Hensen's cells, Claudius cells, inner sulcus cells, outer sulcus cells, spiral prominence cells, root cells, interdental cells, basal cells of the stria vascularis, intermediate cells of the stria vascularis, marginal cells of the stria vascularis, inner hair cells, outer hair cells, vestibular hair cells, and Schwann cells.

As used herein, the term “wild-type” refers to a genotype with the highest frequency for a particular gene in a given organism.

### BRIEF DESCRIPTION OF THE DRAWINGS

FIGS. 1A-1C are a series of single-plane confocal fluorescent images comparing nuclear GFP expression in adult mouse utricles transduced with viral vectors encoding nuclear GFP under control of the SLC6A14v2 (SEQ ID NO: 3; top row) or SLC6A14v3 (SEQ ID NO: 1; bottom row) promoters. Supporting cell nuclei were immunolabeled with antibodies raised against SRY-Box Transcription Factor 2 (Sox2) protein, and hair cell nuclei were immunolabeled with antibodies raised against POU Class 4 Homeobox 3 (Pou4f3) protein. FIG. 1A shows the supporting cell (SC) nuclear layer, FIG. 1B shows the hair cell (HC) nuclear layer, and FIG. 1C shows the mesenchymal layer.

FIGS. 2A-2D are a series of graphs showing quantification of nuclear GFP expression in supporting cells (FIG. 2A), intensity of nuclear GFP expression in supporting cells (FIG. 2B), quantification of nuclear GFP expression in hair cells (FIG. 2C), and quantification in all cells other than supporting cells (FIG. 2D) in adult mouse utricles transduced with viral vectors encoding nuclear GFP under control of the SLC6A14v2 (SEQ ID NO: 3) or SLC6A14v3 (SEQ ID NO: 1) promoters.

FIG. 3 is a map of plasmid P530.

FIG. 4 is a map of plasmid P919.

FIG. 5 is a map of plasmid P990.

FIG. 6 is a map of plasmid P1071.

### DETAILED DESCRIPTION

Described herein are compositions and methods for inducing transgene expression specifically in vestibular supporting cells (VSCs) of the inner ear. The invention features polynucleotides containing regions of the Solute Carrier Family 6 Member 14 (SLC6A14) promoter that are capable of expressing a transgene specifically in VSCs. The invention also features nucleic acid vectors containing said promoters operably linked to polynucleotides encoding polypeptides or RNA molecules. The compositions and methods described herein can be used to express polynucleotides encoding proteins (e.g., therapeutic proteins, reporter proteins, or other proteins of interest) or RNA molecules (e.g., inhibitory RNA molecules) in VSCs, which provide structural and trophic support to vestibular hair cells and can be made to differentiate into hair cells, and, therefore, the compositions described herein can be administered to a subject (such as a mammalian subject, for example, a human) to treat disorders caused by dysfunction of vestibular hair cells, such as vertigo, dizziness, imbalance, bilateral vestibulopathy, oscillopsia, or a balance disorder.

### Supporting Cells

Supporting cells of the vestibular system are specialized epithelial cells that reside in the inner ear. VSCs constitute an anatomically and morphologically homogenous class of cells that mediate critical structural, developmental, and trophic activities necessary for normal vestibular function. VSCs are located within the utricle, saccule, and semicircular canals of the inner ear and act as structural anchors for vestibular hair cells, the primary sensory cells of the peripheral vestibular system involved in the sensation of movement that contributes to a sense of balance and spatial orientation. Formation of synapses onto hair cells from the vestibulocochlear nerve is mediated by neurotrophic factors secreted by VSCs, thereby subserving the establishment and maintenance of proper vestibular function.

Furthermore, VSCs act as important mediators of vestibular hair cell survival, death, and phagocytic clearance by virtue of their control of extracellular and intracellular calcium signaling and formation of phagocytic multicellular structures called phagosomes that maintain the integrity of the sensory epithelium by removing dead or dying hair cells. Damage to vestibular hair cells and genetic mutations that disrupt vestibular hair cell function are implicated in vestibular dysfunction, such as loss of balance and vertigo (e.g., dizziness). Gene therapy has recently emerged as an attractive therapeutic approach for treating vestibular dysfunction; however, the field lacks methods for targeting the nucleic acid vectors used in gene therapy to supporting cells of the vestibular system.

The present invention is based, in part, on the discovery that SLC6A14 is specifically expressed in VSCs of the inner ear. SLC6A14 is a gene encoding a sodium- and chloride-dependent neurotransmitter transporter capable of transporting both neutral and positively charged amino acids in a sodium- and chloride-dependent manner that had not been previously identified as expressed in the inner ear. The SLC6A14 promoter sequences disclosed herein induce gene expression in a VSC-specific manner in the inner ear. The compositions and methods described herein can, thus, be used to express a gene of interest in VSCs (e.g., a gene implicated in vestibular hair cell development, vestibular hair cell fate specification, vestibular hair cell regeneration, vestibular hair cell and/or VSC proliferation, vestibular hair cell innervation, or vestibular hair cell maturation, or a gene known to be disrupted, e.g., mutated, in subjects with vestibular dysfunction) to treat subjects having or at risk of developing vestibular dysfunction (e.g., vertigo, dizziness, loss of balance, bilateral vestibulopathy (e.g., bilateral vestibular hypofunction), oscillopsia, or a balance disorder). Cell type-specific gene expression can improve the safety and efficacy of gene therapy by reducing toxicity associated with off-target expression.

The compositions and methods described herein include an SLC6A14 promoter of SEQ ID NO: 1 that is capable of expressing a transgene in specifically VSCs, or variants thereof, such as nucleic acid sequences that have at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1.

The foregoing nucleic acid sequence is provided in Table 2, below.

TABLE 2
SLC6A14 promoter sequences

| SEQ | Description | |
|---|---|---|
| ID | of promoter | |
| NO: | sequence | Promoter sequence |
| 1 | SLC6A14v3 | ATACACTTATGTATATGTGCGATGTCAGTG |
| | (murine | TGTGTGCATATAAAGTCCCAAACAAGCCTG |
| | promoter) | TATGATATTGACCAACAAGGTCAAGGCAAA |
| | | GTTTTGATACTTTCAGGTCACAACCTCTCC |
| | | GCATCCCTCTCTACTTTGCTCTATCTGCCT |
| | | GAACTCCTGAGGACATGTTTCTACTGCAAA |
| | | TGGAAAATCCTTGTCAGCCAGTGAGGAACA |
| | | AAGGGACTATACATAGATGAAAACTTGGCT |
| | | CTCTGCTGGTTCCTTTGTTTGTATGAATTT |
| | | ATACAATTTGGTAAAACTGCCACCATGTCT |
| | | TACATGGACAGATTGAGTGTAGATTCTTTG |
| | | AATTTTTGATGAAGAGGCGCTGCACTGGTG |
| | | ATCGGAATTGCAGTCTTTCCTCTGTAGGTA |
| | | ACCTGGCTTGTTTCCTTACAGTTTACTTTC |
| | | TAGGCCTCGCCTTTCTCACAGAGTGAAGTC |
| | | CTTTGTTAAGGTTCGAATTTCCCATAAACC |
| | | TGCTCAATAATTTGTTTGTGTTTGGCTTCT |
| | | TTGAAATACTACACAAAGCAATCCTTGTAA |
| | | AAGGCAAAACTATTCCGAAGGCTGAGAAAG |
| | | GAGCTCCAGGACATAGATTCAAAGTCGCTC |
| | | TTTTCAGGTAGAGACAGCTGGGTAATCTTA |
| | | TCTTAACTGGCTACATTTCAAGGTTCCCAA |
| | | TTCAGGGGCTTTCCCCTCTGGGAGCAGCAT |
| | | TCTCTCCGGGTGATGAAGAGCTTTCTAGTG |
| | | AGGAGCAAAACTTTCAGAAAACCGGAGGGC |
| | | CCAGAGCAGTCTGGTCTGTTCACAAAAATT |
| | | ATAGCAAACAAAATAAGCCCGGCGGATTGG |
| | | GTCTCTCCTACCTCCAGCACCAGGGGAGAT |
| | | CAGCACTTGGCCCCAGGACAGAGACCTGAG |
| | | AAGTGAGGTTTGGAAGAAGCCAGGAATCCA |
| | | GGAAAGGAGGCAAGATTGCTAAGGCACCGG |
| | | CACAGCTCTGAGTCAAAAGTTGTCAGTCTT |
| | | CTTTGGCTCTGGCTGCGGAGCTCAATTGCT |
| | | CACAGCCCTGCCCTTTCCTAGGGCTGGGGC |
| | | AAGGAATTGCTACATTCAGGATTACCTGGG |
| | | GGAAAAACCAGAGGCTTGCTTTGGTCCCTT |
| | | CCGGTAATTGAAAGGACTGGCCGTCAGCGA |
| | | GGGGGAGGAGAGAGCTTCCCTCCATAAATG |
| | | GTCCCACCCCTGGGCAAGGTGGCTCACTTT |
| | | GGCAGGTAGCAACCGGGGAGTGTGCACCTG |
| | | CCACCAGTCAAGCTCAGCCAGACTGTGAGA |
| | | AGAGGAGAGGCGAGGCACACCAAGGGATCC |
| | | AGTGAACCaacGACAGATTGAAGTGCCCGA |
| | | ACTTCTTCAAGTGCAGACAGAAGGAGGTAG |
| | | GGTTCTGGAAGTTTCTGGTGGTGTAGGGGA |
| | | GTCAGGAAGGGAAAACAAGGAGGGAGAGTG |
| | | AGTCTTAGTTTTTTGCTTTCTGTAGCTGTT |
| | | CCTTATTTTGCATATTTCTTTCTCTTCAAC |
| | | TCTTTTCAAGTATGCCTGATACGTTGTTCT |
| | | CACGAAGTTGACGTGAAAAACAACTTTCCT |
| | | GCTGGTAGTTAGGAAACTTAGGAGCACCTC |
| | | AACCTGTACCTTGAGAACACCCAGAGAATG |
| | | CTGCTCTTTGTCGTTCTCTATACCGTGTTC |
| | | ATATGCTGCAGGGAAATGCAAAGAATGTAC |
| | | TGTCCTTATCTGACCCTGGGAGCATTCCAT |
| | | AGTCAAGCAGCAGCTATCAGGTTGGGAAAG |
| | | AGCTCCTCTCCAAGGTGTAACAGAAAAGGA |
| | | AAATGTTGATATTTTTCTTGTTTAGAAAGT |
| | | GACAGCTTCATCCGAGAAC |
| 3 | SLC6A14v2 | AAGCTGGGATGTTTCCTCATAGTTTACTTT |
| | (human | CTAGGCCTCATCTTTCTTACAGAGTGTGCT |
| | promoter) | CCTTTGTTAAGGTTAGAATTTCCCATAAAC |
| | | CTGCTCAATAATTTGTTTGTGTTTGGCTTC |
| | | TTTGAAATACTACACAAAGCAATCCCTGTA |
| | | AAAGGCAAAGCTGTCCTGAAGGCTGAGAAA |
| | | GGAGCCTGAGACATAGGCTCCAAGTTGCTC |
| | | TTTTCAGGCAGAGCCAGCTGGGTAATCTTA |
| | | TCTCAGATGGCTGCTTTTCAAGGTGCCCAA |
| | | TTCAGGGGCTTTTCCTCTGGGAGCAGCATT |
| | | TGCCCCAGGGAATCAAGTGCTTTCTAGTCA |
| | | GGGGCAAAACTTTGGGAAATCTGAGGACCC |
| | | AGGGTGGTATGGTCTGTTCAGGAGAATTTT |
| | | GGGGAACAGAATGGCCCCCTTCTCCCTCCA |
| | | GCACTTGTACAGATCAGCACTTGGCCCCAG |
| | | AACAGAGACCAGACTGAGAGGCGAGGTTAG |
| | | GAGGAAACAGGGGACCCAGGAAAGGCGGCT |
| | | AGATTGCAAACGTACCTACACAGCTCTGAG |
| | | TCAAAGGCTGTCAGTCATCTCGGCTCAGAC |
| | | TGCTCTGCTCTCCAGCAGCCCAGCCCTTTC |
| | | CCAGGGCTGGGGCAGGAGATTGCTACATGT |
| | | AGGCTTATCTGGGGAAAAACCAGAGCCTCA |
| | | CTTTAGTCCCTTCCGGTAATTGACACTACT |
| | | GGACACCCAGGAGGGGGAGGAGAGAGCTTC |
| | | TCTTCATAAATGTTCCCACCCCTGGGCAAG |
| | | GTGGCTCACTCTGGCAGGTAGGAACAGGGG |
| | | AGAGTGCACCTGCTACCAGTCAAGCTCAGC |
| | | CAGACTGCAAGAGGAGGCGAGGCG |

### Expression of Exogenous Nucleic Acids in Mammalian Cells

The compositions and methods described herein can be used to induce or increase the expression of proteins encoded by genes of interest (e.g., the wild-type form of a gene implicated in vestibular dysfunction, or a gene involved in vestibular hair cell development, vestibular hair cell fate specification, vestibular hair cell regeneration, vestibular hair cell and/or VSC proliferation, vestibular hair cell innervation, or vestibular hair cell maturation) in VSCs by administering a nucleic acid vector that contains an SLC6A14 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1) operably linked to a nucleic acid sequence that encodes a protein of interest. A wide array of methods has been established for the delivery of proteins to mammalian cells and for the stable expression of genes encoding proteins in mammalian cells. Proteins that can be expressed in connection with the compositions described herein (e.g., when the transgene encoding the protein is operably linked to an SLC6A14 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1) are proteins that are expressed in healthy VSCs (e.g., proteins that play a role in vestibular hair cell development, vestibular hair cell fate specification, vestibular hair cell regeneration, vestibular hair cell and/or VSC proliferation, vestibular hair cell innervation, or vestibular hair cell maturation, or proteins that are deficient in subjects with vestibular dysfunction), or other proteins of interest. Proteins that can be expressed in VSCs using the compositions and methods described herein include Spalt Like Transcription Factor 2 (Sall2), Calmodulin Binding Transcription Activator 1 (Camta1), Hes Related Family BHLH Transcription Factor With YRPW Motif 2 (Hey2), Gata Binding Protein 2 (Gata2), Hes Related Family BHLH Transcription Factor With YRPW Motif 1 (Hey1), Ceramide Synthase 2 (Lass2), SRY-Box 10 (Sox10), GATA Binding Protein 3 (Gata3), Cut Like Homeobox 1 (Cux1), Nuclear Receptor Subfamily 2 Group F Member (Nr2f1), Hes Related Family BHLH Transcription Factor (Hes1), RAR Related Orphan Receptor B (Rorb), Jun Proto-Oncogene AP-1 Transcription Factor Subunit (Jun), Zinc Finger Protein 667 (Zfp667), LIM Homeobox 3 (Lhx3), Nescient Helix-Loop-Helix 1 (Nhlh1), MAX Dimerization Protein 4 (Mxd4), Zinc Finger MIZ-Type Containing 1 (Zmiz1), Myelin Transcription Factor 1 (Myt1), Signal Transducer And Activator Of Transcription 3 (Stat3), BarH Like Homeobox 1 (Barhl1), Thymocyte Selection Associated High Mobility Group Box (Tox), Prospero Homeobox 1 (Prox1), Nuclear Factor I A (Nfia), Thyroid Hormone Receptor Beta (Thrb), MYCL Proto-Oncogene BHLH Transcription Factor (Mycl1), Lysine Demethylase 5A (Kdm5a), CAMP Responsive Element Binding Protein 3 Like 4 (Creb314), ETS Variant 1 (Etv1), Paternally Expressed 3 (Peg3), BTB Domain And CNC Homolog 2 (Bach2), ISL LIM Homeobox 1 (Isl1), Zinc Finger And BTB Domain Containing 38 (Zbtb38), Limb Bud And Heart Development (Lbh), Tubby Bipartite Transcription Factor (Tub), Ubiquitin C (Hmg20), RE1 Silencing Transcription Factor (Rest), Zinc Finger Protein 827 (Zfp827), AF4/FMR2 Family Member 3 (Aff3), PBX/Knotted 1 Homeobox 2 (Pknox2), AT-Rich Interaction Domain 3B (Arid3b), MLX Interacting Protein (Mixip), Zinc Finger Protein (Zfp532), IKAROS Family Zinc Finger 2 (Ikzf2), Spalt Like Transcription Factor 1 (Sall1), SIX Homeobox 2 (Six2), Spalt Like Transcription Factor 3 (Sall3), Lin-28 Homolog B (Lin28b), Regulatory Factor X7 (Rfx7), Brain Derived Neurotrophic Factor (Bdnf), Growth Factor Independent 1 Transcriptional Repressor (Gfi1), POU Class 4 Homeobox 3 (Pou4f3), MYC Proto-Oncogene BHLH Transcription Factor (Myc), β-catenin (Ctnnb1), SRY-Box 2 (Sox2), SRY-Box 4 (Sox4), SRY-Box 11 (Sox11), TEA Domain Transcription Factor 2 (Tead2), Atonal BHLH Transcription Factor 1 (Atoh1), and Atoh1 variants containing substitutions at amino acids 328, 331, and/or 334 (e.g., S328A, S331A, S334A, S328A/S331A, S328A/S334A, S331A/S334A, and S328A/S331A/S334). The polynucleotides (e.g., SLC6A14 promoters) described herein can also be used to express an inhibitory RNA molecule (e.g., a short hairpin RNA (shRNA), an antisense oligonucleotide (ASO)), a nuclease (e.g., CRISPR Associated Protein 9 (Cas9), Transcription Activator-Like Effector Nuclease (TALEN), Zinc Finger Nuclease (ZFN), or guide RNA (gRNA)), or a microRNA in VSCs.

In some embodiments, the protein that is expressed in VSCs using the compositions and methods described herein is Atoh1. An SLC6A14 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1) can be operably linked to a polynucleotide sequence that encodes wild-type Atoh1, or a variant thereof, such as a polynucleotide sequence that encodes a protein having at least 85% sequence identity (e.g., 85%, 90%, 95%, 96%, 97%, 98%, 99%, or more, sequence identity) to the amino acid sequence of wild-type mammalian (e.g., human or mouse) Atoh1 (e.g., SEQ ID NO: 4 or SEQ ID NO: 6). Exemplary Atoh1 amino acid and polynucleotide sequences are listed in Table 3, below.

In some embodiments, the polynucleotide sequence encoding an Atoh1 protein encodes an amino acid sequence that contains one or more conservative amino acid substitutions relative to SEQ ID NO: 4 (e.g., 1, 2, 3, 4, 5, 6, 7, 8, 9, 10, 11, 12, 13, 14, 15, 16, 17, 18, 19, 20 or more conservative amino acid substitutions), provided that the Atoh1 analog encoded retains the therapeutic function of wild-type Atoh1 (e.g., the ability to promote hair cell development). No more than 10% of the amino acids in the Atoh1 protein may be replaced with conservative amino acid substitutions. In some embodiments, the polynucleotide sequence that encodes Atoh1 is any polynucleotide sequence that, by redundancy of the genetic code, encodes SEQ ID NO: 4. The polynucleotide sequence that encodes Atoh1 can be partially or fully codon-optimized for expression (e.g., in human VSCs). Atoh1 may be encoded by a polynucleotide having the sequence of SEQ ID NO: 5. The Atoh1 protein may be a human Atoh1 protein or may be a homolog of the human Atoh1 protein from another mammalian species (e.g., mouse, rat, cow, horse, goat, sheep, donkey, cat, dog, rabbit, guinea pig, or other mammal).

TABLE 3
Atoh1 sequences

| SEQ | Description of | |
|---|---|---|
| ID | promoter | |
| NO: | sequence | Sequence |
| 4 | Human Atoh1 amino | MSRLLHAEEWAEVKELGDHH |
| | acid sequence, | RQPQPHHLPQPPPPPQPPAT |
| | RefSeq accession | LQAREHPVYPPELSLLDSTD |
| | number | PRAWLAPTLQGICTARAAQY |
| | NP_005163.1 | LLHSPELGASEAAAPRDEVD |
| | | GRGELVRRSSGGASSSKSPG |
| | | PVKVREQLCKLKGGVVVDEL |
| | | GCSRQRAPSSKQVNGVQKQR |
| | | RLAANARERRRMHGLNHAFD |
| | | QLRNVIPSFNNDKKLSKYET |
| | | LQMAQIYINALSELLQTPSG |
| | | GEQPPPPPASCKSDHHHLRT |
| | | AASYEGGAGNATAAGAQQAS |
| | | GGSQRPTPPGSCRTRFSAPA |
| | | SAGGYSVQLDALHFSTFEDS |
| | | ALTAMMAQKNLSPSLPGSIL |
| | | QPVQEENSKTSPRSHRSDGE |
| | | FSPHSHYSDSDEAS |
| 5 | Human ATOH1 | ATGTCCCGCCTGCTGCATGC |
| | protein coding | AGAAGAGTGGGCTGAAGTGA |
| | sequence, also | AGGAGTTGGGAGACCACCAT |
| | documented under | CGCCAGCCCCAGCCGCATCA |
| | RefSeq accession | TCTCCCGCAACCGCCGCCGC |
| | number | CGCCGCAGCCACCTGCAACT |
| | NM_005172.2 | TTGCAGGCGAGAGAGCATCC |
| | | CGTCTACCCGCCTGAGCTGT |
| | | CCCTCCTGGACAGCACCGAC |
| | | CCACGCGCCTGGCTGGCTCC |
| | | CACTTTGCAGGGCATCTGCA |
| | | CGGCACGCGCCGCCCAGTAT |
| | | TTGCTACATTCCCCGGAGCT |
| | | GGGTGCCTCAGAGGCCGCTG |
| | | CGCCCCGGGACGAGGTGGAC |
| | | GGCCGGGGGGAGCTGGTAAG |
| | | GAGGAGCAGCGGCGGTGCCA |
| | | GCAGCAGCAAGAGCCCCGGG |
| | | CCGGTGAAAGTGCGGGAACA |
| | | GCTGTGCAAGCTGAAAGGCG |
| | | GGGTGGTGGTAGACGAGCTG |
| | | GGCTGCAGCCGCCAACGGGC |
| | | CCCTTCCAGCAAACAGGTGA |
| | | ATGGGGTGCAGAAGCAGAGA |
| | | CGGCTAGCAGCCAACGCCAG |
| | | GGAGCGGCGCAGGATGCATG |
| | | GGCTGAACCACGCCTTCGAC |
| | | CAGCTGCGCAATGTTATCCC |
| | | GTCGTTCAACAACGACAAGA |
| | | AGCTGTCCAAATATGAGACC |
| | | CTGCAGATGGCCCAAATCTA |
| | | CATCAACGCCTTGTCCGAGC |
| | | TGCTACAAACGCCCAGCGGA |
| | | GGGGAACAGCCACCGCCGCC |
| | | TCCAGCCTCCTGCAAAAGCG |
| | | ACCACCACCACCTTCGCACC |
| | | GCGGCCTCCTATGAAGGGGG |
| | | CGCGGGCAACGCGACCGCAG |
| | | CTGGGGCTCAGCAGGCTTCC |
| | | GGAGGGAGCCAGCGGCCGAC |
| | | CCCGCCCGGGAGTTGCCGGA |
| | | CTCGCTTCTCAGCCCCAGCT |
| | | TCTGCGGGAGGGTACTCGGT |
| | | GCAGCTGGACGCTCTGCACT |
| | | TCTCGACTTTCGAGGACAGC |
| | | GCCCTGACAGCGATGATGGC |
| | | GCAAAAGAATTTGTCTCCTT |
| | | CTCTCCCCGGGAGCATCTTG |
| | | CAGCCAGTGCAGGAGGAAAA |
| | | CAGCAAAACTTCGCCTCGGT |
| | | CCCACAGAAGCGACGGGGAA |
| | | TTTTCCCCCCATTCCCATTA |
| | | CAGTGACTCGGATGAGGCAA |
| | | GT |
| 6 | Murine Atoh1 | MSRLLHAEEWAEVKELGDHH |
| | amino | RHPQPHHVPPLTPQPPATLQ |
| | acid sequence, | ARDLPVYPAELSLLDSTDPR |
| | UniProt P48985 | AWLTPTLQGLCTARAAQYLL |
| | | HSPELGASEAAAPRDEADSQ |
| | | GELVRRSGCGGLSKSPGPVK |
| | | VREQLCKLKGGVVVDELGCS |
| | | RQRAPSSKQVNGVQKQRRLA |
| | | ANARERRRMHGLNHAFDQLR |
| | | NVIPSFNNDKKLSKYETLQM |
| | | AQIYINALSELLQTPNVGEQ |
| | | PPPPTASCKNDHHHLRTASS |
| | | YEGGAGASAVAGAQPAPGGG |
| | | PRPTPPGPCRTRFSGPASSG |
| | | GYSVQLDALHFPAFEDRALT |
| | | AMMAQKDLSPSLPGGILQPV |
| | | QEDNSKTSPRSHRSDGEFSP |
| | | HSHYSDSDEAS |
| 7 | Murine ATOH1 | ATGTCCCGCCTGCTGCATGC |
| | protein coding | AGAAGAGTGGGCTGAGGTAA |
| | sequence, also | AAGAGTTGGGGGACCACCAT |
| | documented under | CGCCATCCCCAGCCGCACCA |
| | RefSeq accession | CGTCCCGCCGCTGACGCCAC |
| | number | AGCCACCTGCTACCCTGCAG |
| | NM_007500.5 | GCGAGAGACCTTCCCGTCTA |
| | | CCCGGCAGAACTGTCCCTCC |
| | | TGGATAGCACCGACCCACGC |
| | | GCCTGGCTGACTCCCACTTT |
| | | GCAGGGCCTCTGCACGGCAC |
| | | GCGCCGCCCAGTATCTGCTG |
| | | CATTCTCCCGAGCTGGGTGC |
| | | CTCCGAGGCCGCGGCGCCCC |
| | | GGGACGAGGCTGACAGCCAG |
| | | GGTGAGCTGGTAAGGAGAAG |
| | | CGGCTGTGGCGGCCTCAGCA |
| | | AGAGCCCCGGGCCCGTCAAA |
| | | GTACGGGAACAGCTGTGCAA |
| | | GCTGAAGGGTGGGGTTGTAG |
| | | TGGACGAGCTTGGCTGCAGC |
| | | CGCCAGCGAGCCCCTTCCAG |
| | | CAAACAGGTGAATGGGGTAC |
| | | AGAAGCAAAGGAGGCTGGCA |
| | | GCAAACGCAAGGGAACGGCG |
| | | CAGGATGCACGGGCTGAACC |
| | | ACGCCTTCGACCAGCTGCGC |
| | | AACGTTATCCCGTCCTTCAA |
| | | CAACGACAAGAAGCTGTCCA |
| | | AATATGAGACCCTACAGATG |
| | | GCCCAGATCTACATCAACGC |
| | | TCTGTCGGAGTTGCTGCAGA |
| | | CTCCCAATGTCGGAGAGCAA |
| | | CCGCCGCCGCCCACAGCTTC |
| | | CTGCAAAAATGACCACCATC |
| | | ACCTTCGCACCGCCTCCTCC |
| | | TATGAAGGAGGTGCGGGCGC |
| | | CTCTGCGGTAGCTGGGGCTC |
| | | AGCCAGCCCCGGGAGGGGGC |
| | | CCGAGACCTACCCCGCCCGG |
| | | GCCTTGCCGGACTCGCTTCT |
| | | CAGGCCCAGCTTCCTCTGGG |
| | | GGTTACTCGGTGCAGCTGGA |
| | | CGCTTTGCACTTCCCAGCCT |
| | | TCGAGGACAGGGCCCTAACA |
| | | GCGATGATGGCACAGAAGGA |
| | | CCTGTCGCCTTCGCTGCCCG |
| | | GGGGCATCCTGCAGCCTGTA |
| | | CAGGAGGACAACAGCAAAAC |
| | | ATCTCCCAGATCCCACAGAA |
| | | GTGACGGAGAGTTTTCCCCC |
| | | CACTCTCATTACAGTGACTC |
| | | TGATGAGGCCAGT |

### Polynucleotides Encoding Proteins of Interest

One platform that can be used to achieve therapeutically effective intracellular concentrations of proteins of interest in mammalian cells is via the stable expression of the gene encoding the protein of interest (e.g., by integration into the nuclear or mitochondrial genome of a mammalian cell, or by episomal concatemer formation in the nucleus of a mammalian cell). The gene is a polynucleotide that encodes the primary amino acid sequence of the corresponding protein. In order to introduce exogenous genes into a mammalian cell, genes can be incorporated into a vector. Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

Proteins of interest can also be introduced into a mammalian cell by targeting a vector containing a gene encoding a protein of interest to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding a protein of interest by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Examples of mammalian promoters have been described in Smith, et al., Mol. Sys. Biol., 3:73, online publication, the disclosure of which is incorporated herein by reference. The promoter used in the methods and compositions described herein is an SLC6A14 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1).

Once a polynucleotide encoding a protein of interest has been incorporated into the nuclear DNA of a mammalian cell, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms include tetracycline and doxycycline. These reagents are commercially available (Life Technologies, Carlsbad, CA) and can be administered to a mammalian cell in order to promote gene expression according to established protocols.

Other DNA sequence elements that may be included in polynucleotides for use in the compositions and methods described herein include enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode a protein of interest and additionally include a mammalian enhancer sequence. Many enhancer sequences are now known from mammalian genes, and examples include enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples include the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription include the CMV enhancer and RSV enhancer. An enhancer may be spliced into a vector containing a polynucleotide encoding a protein of interest, for example, at a position 5′ or 3′ to this gene. In a preferred orientation, the enhancer is positioned at the 5′ side of the promoter, which in turn is located 5′ relative to the polynucleotide encoding a protein of interest.

The nucleic acid vectors containing an SLC6A14 promoter described herein may include a WPRE. The WPRE acts at the mRNA level, by promoting nuclear export of transcripts and/or by increasing the efficiency of polyadenylation of the nascent transcript, thus increasing the total amount of mRNA in the cell. The addition of the WPRE to a vector can result in a substantial improvement in the level of transgene expression from several different promoters, both in vitro and in vivo. In some embodiments of the compositions and methods described herein, the WPRE has the sequence:

(SEQ ID NO: 8)
GATCCAATCAACCTCTGGATTACAAAATTTGTGAAAGATTGACTG
GTATTCTTAACTATGTTGCTCCTTTTACGCTATGTGGATACGCTG
CTTTAATGCCTTTGTATCATGCTATTGCTTCCCGTATGGCTTTCA
TTTTCTCCTCCTTGTATAAATCCTGGTTGCTGTCTCTTTATGAGG
AGTTGTGGCCCGTTGTCAGGCAACGTGGCGTGGTGTGCACTGTGT
TTGCTGACGCAACCCCCACTGGTTGGGGCATTGCCACCACCTGTC
AGCTCCTTTCCGGGACTTTCGCTTTCCCCCTCCCTATTGCCACGG
CGGAACTCATCGCCGCCTGCCTTGCCCGCTGCTGGACAGGGGCTC
GGCTGTTGGGCACTGACAATTCCGTGGTGTTGTCGGGGAAATCAT
CGTCCTTTCCTTGGCTGCTCGCCTGTGTTGCCACCTGGATTCTGC
GCGGGACGTCCTTCTGCTACGTCCCTTCGGCCCTCAATCCAGCGG
ACCTTCCTTCCCGCGGCCTGCTGCCGGCTCTGCGGCCTCTTCCGC
GTCTTCGA.

In other embodiments, the WPRE has the sequence:

(SEQ ID NO: 9)
AATCAACCTCTGGATTACAAAATTTGTGAAAGATTGACTGGTATT
CTTAACTATGTTGCTCCTTTTACGCTATGTGGATACGCTGCTTTA
ATGCCTTTGTATCATGCTATTGCTTCCCGTATGGCTTTCATTTTC
TCCTCCTTGTATAAATCCTGGTTAGTTCTTGCCACGGCGGAACTC
ATCGCCGCCTGCCTTGCCCGCTGCTGGACAGGGGCTCGGCTGTTG
GGCACTGACAATTCCGTGGTGTTTATTTGTGAAATTTGTGATGCT
ATTGCTTTATTTGTAACCATCTAGCTTTATTTGTGAAATTTGTGA
TGCTATTGCTTTATTTGTAACCATTATAAGCTGCAATAAACAAGT
TAACAACAACAATTGCATTCATTTTATGTTTCAGGTTCAGGGGGA
GATGTGGGAGGTTTTTTAAA.

In some embodiments, the nucleic acid vectors containing an SLC6A14 promoter described herein include a reporter sequence, which can be useful in verifying the expression of a gene operably linked to an SLC6A14 promoter in VSCs or which can be used to determine or confirm the vestibular supporting cell-specificity of the promoter. Reporter sequences that may be provided in a transgene include DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements that drive their expression, such as an SLC6A14 promoter, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for ß-galactosidase activity. Where the transgene is green fluorescent protein or luciferase, the vector carrying the signal may be measured visually by color or light production in a luminometer.

Transfer plasmids that may be used to produce nucleic acid vectors (e.g., AAV vectors) for use in the compositions and methods described herein are provided in Table 4. A transfer plasmid (e.g., a plasmid containing a DNA sequence to be delivered by a nucleic acid vector, e.g., to be delivered by an AAV) may be co-delivered into producer cells with a helper plasmid (e.g., a plasmid providing proteins necessary for AAV manufacture) and a rep/cap plasmid (e.g., a plasmid that provides AAV capsid proteins and proteins that insert the transfer plasmid DNA sequence into the capsid shell) to produce a nucleic acid vector (e.g., an AAV vector) for administration. The transfer plasmids provided in Table 4 can be used to produce nucleic acid vectors (e.g., AAV vectors) containing an SLC6A14 promoter operably linked to a transgene, such as a polynucleotide encoding Atoh1 (murine (SEQ ID NO: 11) or human (SEQ ID NO: 10) Atoh1) or a polynucleotide encoding GFP (SEQ ID NO: 2).

TABLE 4
Transfer plasmids

| SEQ | | |
|---|---|---|
| ID | | |
| NO. | Description | Plasmid Sequence |
| 2 | Plasmid P919 | CTGCGCGCTCGCTCGCTCAC |
| | 5′ ITR at | TGAGGCCGCCCGGGCAAAGC |
| | positions 1-130 | CCGGGCGTCGGGCGACCTTT |
| | SLC6A14v3 promoter | GGTCGCCCGGCCTCAGTGAG |
| | at positions | CGAGCGAGCGCGCAGAGAGG |
| | 219-1977 | GAGTGGCCAACTCCATCACT |
| | H2B sequence at | AGGGGTTCCTTGTAGTTAAT |
| | positions 1992-2369 | GATTAACCCGCCATGCTACT |
| | eGFP sequence at | TATCTACGTAGCCATGCTCT |
| | positions 2388-3107 | AGGAAGATCGGAATTCGCCC |
| | WPRE at positions | TTAAGCTAGCGGCGCGCCAT |
| | 3116-3663 | ACACTTATGTATATGTGCGA |
| | polyA at positions | TGTCAGTGTGTGTGCATATA |
| | 3748-3955 | AAGTCCCAAACAAGCCTGTA |
| | 3′ ITR at positions | TGATATTGACCAACAAGGTC |
| | 4043-4172 | AAGGCAAAGTTTTGATACTT |
| | | TCAGGTCACAACCTCTCCGC |
| | | ATCCCTCTCTACTTTGCTCT |
| | | ATCTGCCTGAACTCCTGAGG |
| | | ACATGTTTCTACTGCAAATG |
| | | GAAAATCCTTGTCAGCCAGT |
| | | GAGGAACAAAGGGACTATAC |
| | | ATAGATGAAAACTTGGCTCT |
| | | CTGCTGGTTCCTTTGTTTGT |
| | | ATGAATTTATACAATTTGGT |
| | | AAAACTGCCACCATGTCTTA |
| | | CATGGACAGATTGAGTGTAG |
| | | ATTCTTTGAATTTTTGATGA |
| | | AGAGGCGCTGCACTGGTGAT |
| | | CGGAATTGCAGTCTTTCCTC |
| | | TGTAGGTAACCTGGCTTGTT |
| | | TCCTTACAGTTTACTTTCTA |
| | | GGCCTCGCCTTTCTCACAGA |
| | | GTGAAGTCCTTTGTTAAGGT |
| | | TCGAATTTCCCATAAACCTG |
| | | CTCAATAATTTGTTTGTGTT |
| | | TGGCTTCTTTGAAATACTAC |
| | | ACAAAGCAATCCTTGTAAAA |
| | | GGCAAAACTATTCCGAAGGC |
| | | TGAGAAAGGAGCTCCAGGAC |
| | | ATAGATTCAAAGTCGCTCTT |
| | | TTCAGGTAGAGACAGCTGGG |
| | | TAATCTTATCTTAACTGGCT |
| | | ACATTTCAAGGTTCCCAATT |
| | | CAGGGGCTTTCCCCTCTGGG |
| | | AGCAGCATTCTCTCCGGGTG |
| | | ATGAAGAGCTTTCTAGTGAG |
| | | GAGCAAAACTTTCAGAAAAC |
| | | CGGAGGGCCCAGAGCAGTCT |
| | | GGTCTGTTCACAAAAATTAT |
| | | AGCAAACAAAATAAGCCCGG |
| | | CGGATTGGGTCTCTCCTACC |
| | | TCCAGCACCAGGGGAGATCA |
| | | GCACTTGGCCCCAGGACAGA |
| | | GACCTGAGAAGTGAGGTTTG |
| | | GAAGAAGCCAGGAATCCAGG |
| | | AAAGGAGGCAAGATTGCTAA |
| | | GGCACCGGCACAGCTCTGAG |
| | | TCAAAAGTTGTCAGTCTTCT |
| | | TTGGCTCTGGCTGCGGAGCT |
| | | CAATTGCTCACAGCCCTGCC |
| | | CTTTCCTAGGGCTGGGGCAA |
| | | GGAATTGCTACATTCAGGAT |
| | | TACCTGGGGGAAAAACCAGA |
| | | GGCTTGCTTTGGTCCCTTCC |
| | | GGTAATTGAAAGGACTGGCC |
| | | GTCAGCGAGGGGGAGGAGAG |
| | | AGCTTCCCTCCATAAATGGT |
| | | CCCACCCCTGGGCAAGGTGG |
| | | CTCACTTTGGCAGGTAGCAA |
| | | CCGGGGAGTGTGCACCTGCC |
| | | ACCAGTCAAGCTCAGCCAGA |
| | | CTGTGAGAAGAGGAGAGGCG |
| | | AGGCACACCAAGGGATCCAG |
| | | TGAACCAACGACAGATTGAA |
| | | GTGCCCGAACTTCTTCAAGT |
| | | GCAGACAGAAGGAGGTAGGG |
| | | TTCTGGAAGTTTCTGGTGGT |
| | | GTAGGGGAGTCAGGAAGGGA |
| | | AAACAAGGAGGGAGAGTGAG |
| | | TCTTAGTTTTTTGCTTTCTG |
| | | TAGCTGTTCCTTATTTTGCA |
| | | TATTTCTTTCTCTTCAACTC |
| | | TTTTCAAGTATGCCTGATAC |
| | | GTTGTTCTCACGAAGTTGAC |
| | | GTGAAAAACAACTTTCCTGC |
| | | TGGTAGTTAGGAAACTTAGG |
| | | AGCACCTCAACCTGTACCTT |
| | | GAGAACACCCAGAGAATGCT |
| | | GCTCTTTGTCGTTCTCTATA |
| | | CCGTGTTCATATGCTGCAGG |
| | | GAAATGCAAAGAATGTACTG |
| | | TCCTTATCTGACCCTGGGAG |
| | | CATTCCATAGTCAAGCAGCA |
| | | GCTATCAGGTTGGGAAAGAG |
| | | CTCCTCTCCAAGGTGTAACA |
| | | GAAAAGGAAAATGTTGATAT |
| | | TTTTCTTGTTTAGAAAGTGA |
| | | CAGCTTCATCCGAGAACGCG |
| | | GCCGCGCCACCATGCCAGAG |
| | | CCAGCGAAGTCTGCTCCCGC |
| | | CCCGAAAAAGGGCTCCAAGA |
| | | AGGCGGTGACTAAGGCGCAG |
| | | AAGAAAGGCGGCAAGAAGCG |
| | | CAAGCGCAGCCGCAAGGAGA |
| | | GCTATTCCATCTATGTGTAC |
| | | AAGGTTCTGAAGCAGGTCCA |
| | | CCCTGACACCGGCATTTCGT |
| | | CCAAGGCCATGGGCATCATG |
| | | AATTCGTTTGTGAACGACAT |
| | | TTTCGAGCGCATCGCAGGTG |
| | | AGGCTTCCCGCCTGGCGCAT |
| | | TACAACAAGCGCTCGACCAT |
| | | CACCTCCAGGGAGATCCAGA |
| | | CGGCCGTGCGCCTGCTGCTG |
| | | CCTGGGGAGTTGGCCAAGCA |
| | | CGCCGTGTCCGAGGGTACTA |
| | | AGGCCATCACCAAGTACACC |
| | | AGCGCTAAGGATCCACCGGT |
| | | CGCCACCATGGTGAGCAAGG |
| | | GCGAGGAGCTGTTCACCGGG |
| | | GTGGTGCCCATCCTGGTCGA |
| | | GCTGGACGGCGACGTAAACG |
| | | GCCACAAGTTCAGCGTGTCC |
| | | GGCGAGGGCGAGGGCGATGC |
| | | CACCTACGGCAAGCTGACCC |
| | | TGAAGTTCATCTGCACCACC |
| | | GGCAAGCTGCCCGTGCCCTG |
| | | GCCCACCCTCGTGACCACCC |
| | | TGACCTACGGCGTGCAGTGC |
| | | TTCAGCCGCTACCCCGACCA |
| | | CATGAAGCAGCACGACTTCT |
| | | TCAAGTCCGCCATGCCCGAA |
| | | GGCTACGTCCAGGAGCGCAC |
| | | CATCTTCTTCAAGGACGACG |
| | | GCAACTACAAGACCCGCGCC |
| | | GAGGTGAAGTTCGAGGGCGA |
| | | CACCCTGGTGAACCGCATCG |
| | | AGCTGAAGGGCATCGACTTC |
| | | AAGGAGGACGGCAACATCCT |
| | | GGGGCACAAGCTGGAGTACA |
| | | ACTACAACAGCCACAACGTC |
| | | TATATCATGGCCGACAAGCA |
| | | GAAGAACGGCATCAAGGTGA |
| | | ACTTCAAGATCCGCCACAAC |
| | | ATCGAGGACGGCAGCGTGCA |
| | | GCTCGCCGACCACTACCAGC |
| | | AGAACACCCCCATCGGCGAC |
| | | GGCCCCGTGCTGCTGCCCGA |
| | | CAACCACTACCTGAGCACCC |
| | | AGTCCGCCCTGAGCAAAGAC |
| | | CCCAACGAGAAGCGCGATCA |
| | | CATGGTCCTGCTGGAGTTCG |
| | | TGACCGCCGCCGGGATCACT |
| | | CTCGGCATGGACGAGCTGTA |
| | | CAAGTAATAAGCTTGGATCC |
| | | AATCAACCTCTGGATTACAA |
| | | AATTTGTGAAAGATTGACTG |
| | | GTATTCTTAACTATGTTGCT |
| | | CCTTTTACGCTATGTGGATA |
| | | CGCTGCTTTAATGCCTTTGT |
| | | ATCATGCTATTGCTTCCCGT |
| | | ATGGCTTTCATTTTCTCCTC |
| | | CTTGTATAAATCCTGGTTGC |
| | | TGTCTCTTTATGAGGAGTTG |
| | | TGGCCCGTTGTCAGGCAACG |
| | | TGGCGTGGTGTGCACTGTGT |
| | | TTGCTGACGCAACCCCCACT |
| | | GGTTGGGGCATTGCCACCAC |
| | | CTGTCAGCTCCTTTCCGGGA |
| | | CTTTCGCTTTCCCCCTCCCT |
| | | ATTGCCACGGCGGAACTCAT |
| | | CGCCGCCTGCCTTGCCCGCT |
| | | GCTGGACAGGGGCTCGGCTG |
| | | TTGGGCACTGACAATTCCGT |
| | | GGTGTTGTCGGGGAAATCAT |
| | | CGTCCTTTCCTTGGCTGCTC |
| | | GCCTGTGTTGCCACCTGGAT |
| | | TCTGCGCGGGACGTCCTTCT |
| | | GCTACGTCCCTTCGGCCCTC |
| | | AATCCAGCGGACCTTCCTTC |
| | | CCGCGGCCTGCTGCCGGCTC |
| | | TGCGGCCTCTTCCGCGTCTT |
| | | CGAACAATTGCATCGGACAC |
| | | ATCTTGGCGTTTTACAACGT |
| | | CGTGACTGGGAAAACCCTGG |
| | | CGTTACCCAACTTAAGATCT |
| | | GCCTCGACTGTGCCTTCTAG |
| | | TTGCCAGCCATCTGTTGTTT |
| | | GCCCCTCCCCCGTGCCTTCC |
| | | TTGACCCTGGAAGGTGCCAC |
| | | TCCCACTGTCCTTTCCTAAT |
| | | AAAATGAGGAAATTGCATCG |
| | | CATTGTCTGAGTAGGTGTCA |
| | | TTCTATTCTGGGGGGTGGGG |
| | | TGGGGCAGGACAGCAAGGGG |
| | | GAGGATTGGGAAGACAATAG |
| | | CAGGCATGCTGGGGACTCGA |
| | | GTTAAGGGCGAATTCCCGAT |
| | | AAGGATOTTCCTAGAGCATG |
| | | GCTACGTAGATAAGTAGCAT |
| | | GGGGGTTAATCATTAACTAC |
| | | AAGGAACCCCTAGTGATGGA |
| | | GTTGGCCACTCCCTCTCTGC |
| | | GCGCTCGCTCGCTCACTGAG |
| | | GCCGGGCGACCAAAGGTCGC |
| | | CCGACGCCCGGGCTTTGCCC |
| | | GGGCGGCCTCAGTGAGCGAG |
| | | CGAGCGCGCAGCCTTAATTA |
| | | ACCTAATTCACTGGCCGTCG |
| | | TTTTACAACGTCGTGACTGG |
| | | GAAAACCCTGGCGTTACCCA |
| | | ACTTAATCGCCTTGCAGCAC |
| | | ATCCCCCTTTCGCCAGCTGG |
| | | CGTAATAGCGAAGAGGCCCG |
| | | CACCGATCGCCCTTCCCAAC |
| | | AGTTGCGCAGCCTGAATGGC |
| | | GAATGGGACGCGCCCTGTAG |
| | | CGGCGCATTAAGCGCGGCGG |
| | | GTGTGGTGGTTACGCGCAGC |
| | | GTGACCGCTACACTTGCCAG |
| | | CGCCCTAGCGCCCGCTCCTT |
| | | TCGCTTTCTTCCCTTCCTTT |
| | | CTCGCCACGTTCGCCGGCTT |
| | | TCCCCGTCAAGCTCTAAATC |
| | | GGGGGCTCCCTTTAGGGTTC |
| | | CGATTTAGTGCTTTACGGCA |
| | | CCTCGACCCCAAAAAACTTG |
| | | ATTAGGGTGATGGTTCACGT |
| | | AGTGGGCCATCGCCCTGATA |
| | | GACGGTTTTTCGCCCTTTGA |
| | | CGTTGGAGTCCACGTTCTTT |
| | | AATAGTGGACTCTTGTTCCA |
| | | AACTGGAACAACACTCAACC |
| | | CTATCTCGGTCTATTCTTTT |
| | | GATTTATAAGGGATTTTGCC |
| | | GATTTCGGCCTATTGGTTAA |
| | | AAAATGAGCTGATTTAACAA |
| | | AAATTTAACGCGAATTTTAA |
| | | CAAAATATTAACGCTTACAA |
| | | TTTAGGTGGCACTTTTCGGG |
| | | GAAATGTGCGCGGAACCCCT |
| | | ATTTGTTTATTTTTCTAAAT |
| | | ACATTCAAATATGTATCCGC |
| | | TCATGAGACAATAACCCTGA |
| | | TAAATGCTTCAATAATATTG |
| | | AAAAAGGAAGAGTATGAGTA |
| | | TTCAACATTTCCGTGTCGCC |
| | | CTTATTCCCTTTTTTGCGGC |
| | | ATTTTGCCTTCCTGTTTTTG |
| | | CTCACCCAGAAACGCTGGTG |
| | | AAAGTAAAAGATGCTGAAGA |
| | | TCAGTTGGGTGCACGAGTGG |
| | | GTTACATCGAACTGGATCTC |
| | | AACAGCGGTAAGATCCTTGA |
| | | GAGTTTTCGCCCCGAAGAAC |
| | | GTTTTCCAATGATGAGCACT |
| | | TTTAAAGTTCTGCTATGTGG |
| | | CGCGGTATTATCCCGTATTG |
| | | ACGCCGGGCAAGAGCAACTC |
| | | GGTCGCCGCATACACTATTC |
| | | TCAGAATGACTTGGTTGAGT |
| | | ACTCACCAGTCACAGAAAAG |
| | | CATCTTACGGATGGCATGAC |
| | | AGTAAGAGAATTATGCAGTG |
| | | CTGCCATAACCATGAGTGAT |
| | | AACACTGCGGCCAACTTACT |
| | | TCTGACAACGATCGGAGGAC |
| | | CGAAGGAGCTAACCGCTTTT |
| | | TTGCACAACATGGGGGATCA |
| | | TGTAACTCGCCTTGATCGTT |
| | | GGGAACCGGAGCTGAATGAA |
| | | GCCATACCAAACGACGAGCG |
| | | TGACACCACGATGCCTGTAG |
| | | CAATGGCAACAACGTTGCGC |
| | | AAACTATTAACTGGCGAACT |
| | | ACTTACTCTAGCTTCCCGGC |
| | | AACAATTAATAGACTGGATG |
| | | GAGGCGGATAAAGTTGCAGG |
| | | ACCACTTCTGCGCTCGGCCC |
| | | TTCCGGCTGGCTGGTTTATT |
| | | GCTGATAAATCTGGAGCCGG |
| | | TGAGCGTGGGTCTCGCGGTA |
| | | TCATTGCAGCACTGGGGCCA |
| | | GATGGTAAGCCCTCCCGTAT |
| | | CGTAGTTATCTACACGACGG |
| | | GGAGTCAGGCAACTATGGAT |
| | | GAACGAAATAGACAGATCGC |
| | | TGAGATAGGTGCCTCACTGA |
| | | TTAAGCATTGGTAACTGTCA |
| | | GACCAAGTTTACTCATATAT |
| | | ACTTTAGATTGATTTAAAAC |
| | | TTCATTTTTAATTTAAAAGG |
| | | ATCTAGGTGAAGATCCTTTT |
| | | TGATAATCTCATGACCAAAA |
| | | TCCCTTAACGTGAGTTTTCG |
| | | TTCCACTGAGCGTCAGACCC |
| | | CGTAGAAAAGATCAAAGGAT |
| | | CTTCTTGAGATCCTTTTTTT |
| | | CTGCGCGTAATCTGCTGCTT |
| | | GCAAACAAAAAAACCACCGC |
| | | TACCAGCGGTGGTTTGTTTG |
| | | CCGGATCAAGAGCTACCAAC |
| | | TCTTTTTCCGAAGGTAACTG |
| | | GCTTCAGCAGAGCGCAGATA |
| | | CCAAATACTGTTCTTCTAGT |
| | | GTAGCCGTAGTTAGGCCACC |
| | | ACTTCAAGAACTCTGTAGCA |
| | | CCGCCTACATACCTCGCTCT |
| | | GCTAATCCTGTTACCAGTGG |
| | | CTGCTGCCAGTGGCGATAAG |
| | | TCGTGTCTTACCGGGTTGGA |
| | | CTCAAGACGATAGTTACCGG |
| | | ATAAGGCGCAGCGGTCGGGC |
| | | TGAACGGGGGGTTCGTGCAC |
| | | ACAGCCCAGCTTGGAGCGAA |
| | | CGACCTACACCGAACTGAGA |
| | | TACCTACAGCGTGAGCTATG |
| | | AGAAAGCGCCACGCTTCCCG |
| | | AAGGGAGAAAGGCGGACAGG |
| | | TATCCGGTAAGCGGCAGGGT |
| | | CGGAACAGGAGAGCGCACGA |
| | | GGGAGCTTCCAGGGGGAAAC |
| | | GCCTGGTATCTTTATAGTCC |
| | | TGTCGGGTTTCGCCACCTCT |
| | | GACTTGAGCGTCGATTTTTG |
| | | TGATGCTCGTCAGGGGGGCG |
| | | GAGCCTATGGAAAAACGCCA |
| | | GCAACGCGGCCTTTTTACGG |
| | | TTCCTGGCCTTTTGCTGGCC |
| | | TTTTGCTCACATGTTCTTTC |
| | | CTGCGTTATCCCCTGATTCT |
| | | GTGGATAACCGTATTACCGC |
| | | CTTTGAGTGAGCTGATACCG |
| | | CTCGCCGCAGCCGAACGACC |
| | | GAGCGCAGCGAGTCAGTGAG |
| | | CGAGGAAGCGGAAGAGCGCC |
| | | CAATACGCAAACCGCCTCTC |
| | | CCCGCGCGTTGGCCGATTCA |
| | | TTAATGCAGCTGGCACGACA |
| | | GGTTTCCCGACTGGAAAGCG |
| | | GGCAGTGAGCGCAACGCAAT |
| | | TAATGTGAGTTAGCTCACTC |
| | | ATTAGGCACCCCAGGCTTTA |
| | | CACTTTATGCTTCCGGCTCG |
| | | TATGTTGTGTGGAATTGTGA |
| | | GCGGATAACAATTTCACACA |
| | | GGAAACAGCTATGACCATGA |
| | | TTACGCCAGATTTAATTAAG |
| | | GCCTTAATTAGG |
| 10 | Plasmid P990 | CTGCGCGCTCGCTCGCTCAC |
| | 5′ ITR at | TGAGGCCGCCCGGGCAAAGC |
| | positions 1-130 | CCGGGCGTCGGGCGACCTTT |
| | SLC6A14v3 promoter | GGTCGCCCGGCCTCAGTGAG |
| | at positions | CGAGCGAGCGCGCAGAGAGG |
| | 219-1977 | GAGTGGCCAACTCCATCACT |
| | Human Atoh1 | AGGGGTTCCTTGTAGTTAAT |
| | sequence at | GATTAACCCGCCATGCTACT |
| | positions | TATCTACGTAGCCATGCTCT |
| | 1992-3053 | AGGAAGATCGGAATTCGCCC |
| | WPRE at positions | TTAAGCTAGCGGCGCGCCAT |
| | 3064-3611 | ACACTTATGTATATGTGCGA |
| | polyA at positions | TGTCAGTGTGTGTGCATATA |
| | 3624-3831 | AAGTCCCAAACAAGCCTGTA |
| | 3′ ITR at positions | TGATATTGACCAACAAGGTC |
| | 3919-4048 | AAGGCAAAGTTTTGATACTT |
| | | TCAGGTCACAACCTCTCCGC |
| | | ATCCCTCTCTACTTTGCTCT |
| | | ATCTGCCTGAACTCCTGAGG |
| | | ACATGTTTCTACTGCAAATG |
| | | GAAAATCCTTGTCAGCCAGT |
| | | GAGGAACAAAGGGACTATAC |
| | | ATAGATGAAAACTTGGCTCT |
| | | CTGCTGGTTCCTTTGTTTGT |
| | | ATGAATTTATACAATTTGGT |
| | | AAAACTGCCACCATGTCTTA |
| | | CATGGACAGATTGAGTGTAG |
| | | ATTCTTTGAATTTTTGATGA |
| | | AGAGGCGCTGCACTGGTGAT |
| | | CGGAATTGCAGTCTTTCCTC |
| | | TGTAGGTAACCTGGCTTGTT |
| | | TCCTTACAGTTTACTTTCTA |
| | | GGCCTCGCCTTTCTCACAGA |
| | | GTGAAGTCCTTTGTTAAGGT |
| | | TCGAATTTCCCATAAACCTG |
| | | CTCAATAATTTGTTTGTGTT |
| | | TGGCTTCTTTGAAATACTAC |
| | | ACAAAGCAATCCTTGTAAAA |
| | | GGCAAAACTATTCCGAAGGC |
| | | TGAGAAAGGAGCTCCAGGAC |
| | | ATAGATTCAAAGTCGCTCTT |
| | | TTCAGGTAGAGACAGCTGGG |
| | | TAATCTTATCTTAACTGGCT |
| | | ACATTTCAAGGTTCCCAATT |
| | | CAGGGGCTTTCCCCTCTGGG |
| | | AGCAGCATTCTCTCCGGGTG |
| | | ATGAAGAGCTTTCTAGTGAG |
| | | GAGCAAAACTTTCAGAAAAC |
| | | CGGAGGGCCCAGAGCAGTCT |
| | | GGTCTGTTCACAAAAATTAT |
| | | AGCAAACAAAATAAGCCCGG |
| | | CGGATTGGGTCTCTCCTACC |
| | | TCCAGCACCAGGGGAGATCA |
| | | GCACTTGGCCCCAGGACAGA |
| | | GACCTGAGAAGTGAGGTTTG |
| | | GAAGAAGCCAGGAATCCAGG |
| | | AAAGGAGGCAAGATTGCTAA |
| | | GGCACCGGCACAGCTCTGAG |
| | | TCAAAAGTTGTCAGTCTTCT |
| | | TTGGCTCTGGCTGCGGAGCT |
| | | CAATTGCTCACAGCCCTGCC |
| | | CTTTCCTAGGGCTGGGGCAA |
| | | GGAATTGCTACATTCAGGAT |
| | | TACCTGGGGGAAAAACCAGA |
| | | GGCTTGCTTTGGTCCCTTCC |
| | | GGTAATTGAAAGGACTGGCC |
| | | GTCAGCGAGGGGGAGGAGAG |
| | | AGCTTCCCTCCATAAATGGT |
| | | CCCACCCCTGGGCAAGGTGG |
| | | CTCACTTTGGCAGGTAGCAA |
| | | CCGGGGAGTGTGCACCTGCC |
| | | ACCAGTCAAGCTCAGCCAGA |
| | | CTGTGAGAAGAGGAGAGGCG |
| | | AGGCACACCAAGGGATCCAG |
| | | TGAACCAACGACAGATTGAA |
| | | GTGCCCGAACTTCTTCAAGT |
| | | GCAGACAGAAGGAGGTAGGG |
| | | TTCTGGAAGTTTCTGGTGGT |
| | | GTAGGGGAGTCAGGAAGGGA |
| | | AAACAAGGAGGGAGAGTGAG |
| | | TCTTAGTTTTTTGCTTTCTG |
| | | TAGCTGTTCCTTATTTTGCA |
| | | TATTTCTTTCTCTTCAACTC |
| | | TTTTCAAGTATGCCTGATAC |
| | | GTTGTTCTCACGAAGTTGAC |
| | | GTGAAAAACAACTTTCCTGC |
| | | TGGTAGTTAGGAAACTTAGG |
| | | AGCACCTCAACCTGTACCTT |
| | | GAGAACACCCAGAGAATGCT |
| | | GCTCTTTGTCGTTCTCTATA |
| | | CCGTGTTCATATGCTGCAGG |
| | | GAAATGCAAAGAATGTACTG |
| | | TCCTTATCTGACCCTGGGAG |
| | | CATTCCATAGTCAAGCAGCA |
| | | GCTATCAGGTTGGGAAAGAG |
| | | CTCCTCTCCAAGGTGTAACA |
| | | GAAAAGGAAAATGTTGATAT |
| | | TTTTCTTGTTTAGAAAGTGA |
| | | CAGCTTCATCCGAGAACGCG |
| | | GCCGCGCCACCATGTCCCGC |
| | | CTGCTGCATGCAGAAGAGTG |
| | | GGCTGAAGTGAAGGAGTTGG |
| | | GAGACCACCATCGCCAGCCC |
| | | CAGCCGCATCATCTCCCGCA |
| | | ACCGCCGCCGCCGCCGCAGC |
| | | CACCTGCAACTTTGCAGGCG |
| | | AGAGAGCATCCCGTCTACCC |
| | | GCCTGAGCTGTCCCTCCTGG |
| | | ACAGCACCGACCCACGCGCC |
| | | TGGCTGGCTCCCACTTTGCA |
| | | GGGCATCTGCACGGCACGCG |
| | | CCGCCCAGTATTTGCTACAT |
| | | TCCCCGGAGCTGGGTGCCTC |
| | | AGAGGCCGCTGCGCCCCGGG |
| | | ACGAGGTGGACGGCCGGGGG |
| | | GAGCTGGTAAGGAGGAGCAG |
| | | CGGCGGTGCCAGCAGCAGCA |
| | | AGAGCCCCGGGCCGGTGAAA |
| | | GTGCGGGAACAGCTGTGCAA |
| | | GCTGAAAGGGGGGTGGTGGT |
| | | AGACGAGCTGGGCTGCAGCC |
| | | GCCAACGGGCCCCTTCCAGC |
| | | AAACAGGTGAATGGGGTGCA |
| | | GAAGCAGAGACGGCTAGCAG |
| | | CCAACGCCAGGGAGCGGCGC |
| | | AGGATGCATGGGCTGAACCA |
| | | CGCCTTCGACCAGCTGCGCA |
| | | ATGTTATCCCGTCGTTCAAC |
| | | AACGACAAGAAGCTGTCCAA |
| | | ATATGAGACCCTGCAGATGG |
| | | CCCAAATCTACATCAACGCC |
| | | TTGTCCGAGCTGCTACAAAC |
| | | GCCCAGCGGAGGGGAACAGC |
| | | CACCGCCGCCTCCAGCCTCC |
| | | TGCAAAAGCGACCACCACCA |
| | | CCTTCGCACCGCGGCCTCCT |
| | | ATGAAGGGGGCGCGGGCAAC |
| | | GCGACCGCAGCTGGGGCTCA |
| | | GCAGGCTTCCGGAGGGAGCC |
| | | AGCGGCCGACCCCGCCCGGG |
| | | AGTTGCCGGACTCGCTTCTC |
| | | AGCCCCAGCTTCTGCGGGAG |
| | | GGTACTCGGTGCAGCTGGAC |
| | | GCTCTGCACTTCTCGACTTT |
| | | CGAGGACAGCGCCCTGACAG |
| | | CGATGATGGCGCAAAAGAAT |
| | | TTGTCTCCTTCTCTCCCCGG |
| | | GAGCATCTTGCAGCCAGTGC |
| | | AGGAGGAAAACAGCAAAACT |
| | | TCGCCTCGGTCCCACAGAAG |
| | | CGACGGGGAATTTTCCCCCC |
| | | ATTCCCATTACAGTGACTCG |
| | | GATGAGGCAAGTTAGAAGCT |
| | | TGGATCCAATCAACCTCTGG |
| | | ATTACAAAATTTGTGAAAGA |
| | | TTGACTGGTATTCTTAACTA |
| | | TGTTGCTCCTTTTACGCTAT |
| | | GTGGATACGCTGCTTTAATG |
| | | CCTTTGTATCATGCTATTGO |
| | | TTCCCGTATGGCTTTCATTT |
| | | TCTCCTCCTTGTATAAATCC |
| | | TGGTTGCTGTCTCTTTATGA |
| | | GGAGTTGTGGCCCGTTGTCA |
| | | GGCAACGTGGCGTGGTGTGC |
| | | ACTGTGTTTGCTGACGCAAC |
| | | CCCCACTGGTTGGGGCATTG |
| | | CCACCACCTGTCAGCTCCTT |
| | | TCCGGGACTTTCGCTTTCCC |
| | | CCTCCCTATTGCCACGGCGG |
| | | AACTCATCGCCGCCTGCCTT |
| | | GCCCGCTGCTGGACAGGGGC |
| | | TCGGCTGTTGGGCACTGACA |
| | | ATTCCGTGGTGTTGTCGGGG |
| | | AAATCATCGTCCTTTCCTTG |
| | | GCTGCTCGCCTGTGTTGCCA |
| | | CCTGGATTCTGCGCGGGACG |
| | | TCCTTCTGCTACGTCCCTTC |
| | | GGCCCTCAATCCAGCGGACC |
| | | TTCCTTCCCGCGGCCTGCTG |
| | | CCGGCTCTGCGGCCTCTTCC |
| | | GCGTCTTCGAGATCTGCCTC |
| | | GACTGTGCCTTCTAGTTGCC |
| | | AGCCATCTGTTGTTTGCCCC |
| | | TCCCCCGTGCCTTCCTTGAC |
| | | CCTGGAAGGTGCCACTCCCA |
| | | CTGTCCTTTCCTAATAAAAT |
| | | GAGGAAATTGCATCGCATTG |
| | | TCTGAGTAGGTGTCATTCTA |
| | | TTCTGGGGGGGGGGTGGGGC |
| | | AGGACAGCAAGGGGGAGGAT |
| | | TGGGAAGACAATAGCAGGCA |
| | | TGCTGGGGACTCGAGTTAAG |
| | | GGCGAATTCCCGATAAGGAT |
| | | CTTCCTAGAGCATGGCTACG |
| | | TAGATAAGTAGCATGGCGGG |
| | | TTAATCATTAACTACAAGGA |
| | | ACCCCTAGTGATGGAGTTGG |
| | | CCACTCCCTCTCTGCGCGCT |
| | | CGCTCGCTCACTGAGGCCGG |
| | | GCGACCAAAGGTCGCCCGAC |
| | | GCCCGGGCTTTGCCCGGGCG |
| | | GCCTCAGTGAGCGAGCGAGC |
| | | GCGCAGCCTTAATTAACCTA |
| | | ATTCACTGGCCGTCGTTTTA |
| | | CAACGTCGTGACTGGGAAAA |
| | | CCCTGGCGTTACCCAACTTA |
| | | ATCGCCTTGCAGCACATCCC |
| | | CCTTTCGCCAGCTGGCGTAA |
| | | TAGCGAAGAGGCCCGCACCG |
| | | ATCGCCCTTCCCAACAGTTG |
| | | CGCAGCCTGAATGGCGAATG |
| | | GGACGCGCCCTGTAGCGGCG |
| | | CATTAAGCGCGGCGGGTGTG |
| | | GTGGTTACGCGCAGCGTGAC |
| | | CGCTACACTTGCCAGCGCCC |
| | | TAGCGCCCGCTCCTTTCGCT |
| | | TTCTTCCCTTCCTTTCTCGC |
| | | CACGTTCGCCGGCTTTCCCC |
| | | GTCAAGCTCTAAATCGGGGG |
| | | CTCCCTTTAGGGTTCCGATT |
| | | TAGTGCTTTACGGCACCTCG |
| | | ACCCCAAAAAACTTGATTAG |
| | | GGTGATGGTTCACGTAGTGG |
| | | GCCATCGCCCTGATAGACGG |
| | | TTTTTCGCCCTTTGACGTTG |
| | | GAGTCCACGTTCTTTAATAG |
| | | TGGACTCTTGTTCCAAACTG |
| | | GAACAACACTCAACCCTATC |
| | | TCGGTCTATTCTTTTGATTT |
| | | ATAAGGGATTTTGCCGATTT |
| | | CGGCCTATTGGTTAAAAAAT |
| | | GAGCTGATTTAACAAAAATT |
| | | TAACGCGAATTTTAACAAAA |
| | | TATTAACGCTTACAATTTAG |
| | | GTGGCACTTTTCGGGGAAAT |
| | | GTGCGCGGAACCCCTATTTG |
| | | TTTATTTTTCTAAATACATT |
| | | CAAATATGTATCCGCTCATG |
| | | AGACAATAACCCTGATAAAT |
| | | GCTTCAATAATATTGAAAAA |
| | | GGAAGAGTATGAGCCATATT |
| | | CAACGGGAAACGTCGAGGCC |
| | | GCGATTAAATTCCAACATGG |
| | | ATGCTGATTTATATGGGTAT |
| | | AAATGGGCTCGCGATAATGT |
| | | CGGGCAATCAGGTGCGACAA |
| | | TCTATCGCTTGTATGGGAAG |
| | | CCCGATGCGCCAGAGTTGTT |
| | | TCTGAAACATGGCAAAGGTA |
| | | GCGTTGCCAATGATGTTACA |
| | | GATGAGATGGTCAGACTAAA |
| | | CTGGCTGACGGAATTTATGC |
| | | CTCTTCCGACCATCAAGCAT |
| | | TTTATCCGTACTCCTGATGA |
| | | TGCATGGTTACTCACCACTG |
| | | CGATCCCCGGAAAAACAGCA |
| | | TTCCAGGTATTAGAAGAATA |
| | | TCCTGATTCAGGTGAAAATA |
| | | TTGTTGATGCGCTGGCAGTG |
| | | TTCCTGCGCCGGTTGCATTC |
| | | GATTCCTGTTTGTAATTGTC |
| | | CTTTTAACAGCGATCGCGTA |
| | | TTTCGTCTTGCTCAGGCGCA |
| | | ATCACGAATGAATAACGGTT |
| | | TGGTTGATGCGAGTGATTTT |
| | | GATGACGAGCGTAATGGCTG |
| | | GCCTGTTGAACAAGTCTGGA |
| | | AAGAAATGCATAAACTTTTG |
| | | CCATTCTCACCGGATTCAGT |
| | | CGTCACTCATGGTGATTTCT |
| | | CACTTGATAACCTTATTTTT |
| | | GACGAGGGGAAATTAATAGG |
| | | TTGTATTGATGTTGGACGAG |
| | | TCGGAATCGCAGACCGATAC |
| | | CAGGATCTTGCCATCCTATG |
| | | GAACTGCCTCGGTGAGTTTT |
| | | CTCCTTCATTACAGAAACGG |
| | | CTTTTTCAAAAATATGGTAT |
| | | TGATAATCCTGATATGAATA |
| | | AATTGCAGTTTCATTTGATG |
| | | CTCGATGAGTTTTTCTAACT |
| | | GTCAGACCAAGTTTACTCAT |
| | | ATATACTTTAGATTGATTTA |
| | | AAACTTCATTTTTAATTTAA |
| | | AAGGATCTAGGTGAAGATCC |
| | | TTTTTGATAATCTCATGACC |
| | | AAAATCCCTTAACGTGAGTT |
| | | TTCGTTCCACTGAGCGTCAG |
| | | ACCCCGTAGAAAAGATCAAA |
| | | GGATCTTCTTGAGATCCTTT |
| | | TTTTCTGCGCGTAATCTGCT |
| | | GCTTGCAAACAAAAAAACCA |
| | | CCGCTACCAGCGGTGGTTTG |
| | | TTTGCCGGATCAAGAGCTAC |
| | | CAACTCTTTTTCCGAAGGTA |
| | | ACTGGCTTCAGCAGAGCGCA |
| | | GATACCAAATACTGTTCTTC |
| | | TAGTGTAGCCGTAGTTAGGC |
| | | CACCACTTCAAGAACTCTGT |
| | | AGCACCGCCTACATACCTCG |
| | | CTCTGCTAATCCTGTTACCA |
| | | GTGGCTGCTGCCAGTGGCGA |
| | | TAAGTCGTGTCTTACCGGGT |
| | | TGGACTCAAGACGATAGTTA |
| | | CCGGATAAGGCGCAGCGGTC |
| | | GGGCTGAACGGGGGGTTCGT |
| | | GCACACAGCCCAGCTTGGAG |
| | | CGAACGACCTACACCGAACT |
| | | GAGATACCTACAGCGTGAGC |
| | | TATGAGAAAGCGCCACGCTT |
| | | CCCGAAGGGAGAAAGGCGGA |
| | | CAGGTATCCGGTAAGCGGCA |
| | | GGGTCGGAACAGGAGAGCGC |
| | | ACGAGGGAGCTTCCAGGGGG |
| | | AAACGCCTGGTATCTTTATA |
| | | GTCCTGTCGGGTTTCGCCAC |
| | | CTCTGACTTGAGCGTCGATT |
| | | TTTGTGATGCTCGTCAGGGG |
| | | GGCGGAGCCTATGGAAAAAC |
| | | GCCAGCAACGCGGCCTTTTT |
| | | ACGGTTCCTGGCCTTTTGCT |
| | | GGCCTTTTGCTCACATGTTC |
| | | TTTCCTGCGTTATCCCCTGA |
| | | TTCTGTGGATAACCGTATTA |
| | | CCGCCTTTGAGTGAGCTGAT |
| | | ACCGCTCGCCGCAGCCGAAC |
| | | GACCGAGCGCAGCGAGTCAG |
| | | TGAGCGAGGAAGCGGAAGAG |
| | | CGCCCAATACGCAAACCGCC |
| | | TCTCCCCGCGCGTTGGCCGA |
| | | TTCATTAATGCAGCTGGCAC |
| | | GACAGGTTTCCCGACTGGAA |
| | | AGCGGGCAGTGAGCGCAACG |
| | | CAATTAATGTGAGTTAGCTC |
| | | ACTCATTAGGCACCCCAGGC |
| | | TTTACACTTTATGCTTCCGG |
| | | CTCGTATGTTGTGTGGAATT |
| | | GTGAGCGGATAACAATTTCA |
| | | CACAGGAAACAGCTATGACC |
| | | ATGATTACGCCAGATTTAAT |
| | | TAAGGCCTTAATTAGG |
| 11 | Plasmid P1071 | CTGCGCGCTCGCTCGCTCAC |
| | 5′ ITR at | TGAGGCCGCCCGGGCAAAGC |
| | positions 1-130 | CCGGGCGTCGGGCGACCTTT |
| | SLC6A14v3 | GGTCGCCCGGCCTCAGTGAG |
| | promoter at | CGAGCGAGCGCGCAGAGAGG |
| | positions | GAGTGGCCAACTCCATCACT |
| | 219-1977 | AGGGGTTCCTTGTAGTTAAT |
| | Murine Atoh1 | GATTAACCCGCCATGCTACT |
| | sequence at | TATCTACGTAGCCATGCTCT |
| | positions | AGGAAGATCGGAATTCGCCC |
| | 1992-3044 | TTAAGCTAGCGGCGCGCCAT |
| | WPRE at | ACACTTATGTATATGTGCGA |
| | positions 3055-3602 | TGTCAGTGTGTGTGCATATA |
| | polyA at | AAGTCCCAAACAAGCCTGTA |
| | positions 3615-3822 | TGATATTGACCAACAAGGTC |
| | 3′ ITR at | AAGGCAAAGTTTTGATACTT |
| | positions 3910-4039 | TCAGGTCACAACCTCTCCGC |
| | | ATCCCTCTCTACTTTGCTCT |
| | | ATCTGCCTGAACTCCTGAGG |
| | | ACATGTTTCTACTGCAAATG |
| | | GAAAATCCTTGTCAGCCAGT |
| | | GAGGAACAAAGGGACTATAC |
| | | ATAGATGAAAACTTGGCTCT |
| | | CTGCTGGTTCCTTTGTTTGT |
| | | ATGAATTTATACAATTTGGT |
| | | AAAACTGCCACCATGTCTTA |
| | | CATGGACAGATTGAGTGTAG |
| | | ATTCTTTGAATTTTTGATGA |
| | | AGAGGCGCTGCACTGGTGAT |
| | | CGGAATTGCAGTCTTTCCTC |
| | | TGTAGGTAACCTGGCTTGTT |
| | | TCCTTACAGTTTACTTTCTA |
| | | GGCCTCGCCTTTCTCACAGA |
| | | GTGAAGTCCTTTGTTAAGGT |
| | | TCGAATTTCCCATAAACCTG |
| | | CTCAATAATTTGTTTGTGTT |
| | | TGGCTTCTTTGAAATACTAC |
| | | ACAAAGCAATCCTTGTAAAA |
| | | GGCAAAACTATTCCGAAGGC |
| | | TGAGAAAGGAGCTCCAGGAC |
| | | ATAGATTCAAAGTCGCTCTT |
| | | TTCAGGTAGAGACAGCTGGG |
| | | TAATCTTATCTTAACTGGCT |
| | | ACATTTCAAGGTTCCCAATT |
| | | CAGGGGCTTTCCCCTCTGGG |
| | | AGCAGCATTCTCTCCGGGTG |
| | | ATGAAGAGCTTTCTAGTGAG |
| | | GAGCAAAACTTTCAGAAAAC |
| | | CGGAGGGCCCAGAGCAGTCT |
| | | GGTCTGTTCACAAAAATTAT |
| | | AGCAAACAAAATAAGCCCGG |
| | | CGGATTGGGTCTCTCCTACC |
| | | TCCAGCACCAGGGGAGATCA |
| | | GCACTTGGCCCCAGGACAGA |
| | | GACCTGAGAAGTGAGGTTTG |
| | | GAAGAAGCCAGGAATCCAGG |
| | | AAAGGAGGCAAGATTGCTAA |
| | | GGCACCGGCACAGCTCTGAG |
| | | TCAAAAGTTGTCAGTCTTCT |
| | | TTGGCTCTGGCTGCGGAGCT |
| | | CAATTGCTCACAGCCCTGCC |
| | | CTTTCCTAGGGCTGGGGCAA |
| | | GGAATTGCTACATTCAGGAT |
| | | TACCTGGGGGAAAAACCAGA |
| | | GGCTTGCTTTGGTCCCTTCC |
| | | GGTAATTGAAAGGACTGGCC |
| | | GTCAGCGAGGGGGAGGAGAG |
| | | AGCTTCCCTCCATAAATGGT |
| | | CCCACCCCTGGGCAAGGTGG |
| | | CTCACTTTGGCAGGTAGCAA |
| | | CCGGGGAGTGTGCACCTGCC |
| | | ACCAGTCAAGCTCAGCCAGA |
| | | CTGTGAGAAGAGGAGAGGCG |
| | | AGGCACACCAAGGGATCCAG |
| | | TGAACCAACGACAGATTGAA |
| | | GTGCCCGAACTTCTTCAAGT |
| | | GCAGACAGAAGGAGGTAGGG |
| | | TTCTGGAAGTTTCTGGTGGT |
| | | GTAGGGGAGTCAGGAAGGGA |
| | | AAACAAGGAGGGAGAGTGAG |
| | | TCTTAGTTTTTTGCTTTCTG |
| | | TAGCTGTTCCTTATTTTGCA |
| | | TATTTCTTTCTCTTCAACTC |
| | | TTTTCAAGTATGCCTGATAC |
| | | GTTGTTCTCACGAAGTTGAC |
| | | GTGAAAAACAACTTTCCTGC |
| | | TGGTAGTTAGGAAACTTAGG |
| | | AGCACCTCAACCTGTACCTT |
| | | GAGAACACCCAGAGAATGCT |
| | | GCTCTTTGTCGTTCTCTATA |
| | | CCGTGTTCATATGCTGCAGG |
| | | GAAATGCAAAGAATGTACTG |
| | | TCCTTATCTGACCCTGGGAG |
| | | CATTCCATAGTCAAGCAGCA |
| | | GCTATCAGGTTGGGAAAGAG |
| | | CTCCTCTCCAAGGTGTAACA |
| | | GAAAAGGAAAATGTTGATAT |
| | | TTTTCTTGTTTAGAAAGTGA |
| | | CAGCTTCATCCGAGAACGCG |
| | | GCCGCGCCACCATGTCCCGC |
| | | CTGCTGCATGCAGAAGAGTG |
| | | GGCTGAGGTAAAAGAGTTGG |
| | | GGGACCACCATCGCCATCCC |
| | | CAGCCGCACCACGTCCCGCC |
| | | GCTGACGCCACAGCCACCTG |
| | | CTACCCTGCAGGCGAGAGAC |
| | | CTTCCCGTCTACCCGGCAGA |
| | | ACTGTCCCTCCTGGATAGCA |
| | | CCGACCCACGCGCCTGGCTG |
| | | ACTCCCACTTTGCAGGGCCT |
| | | CTGCACGGCACGCGCCGCCC |
| | | AGTATCTGCTGCATTCTCCC |
| | | GAGCTGGGTGCCTCCGAGGC |
| | | CGCGGCGCCCCGGGACGAGG |
| | | CTGACAGCCAGGGTGAGCTG |
| | | GTAAGGAGAAGCGGCTGTGG |
| | | CGGCCTCAGCAAGAGCCCCG |
| | | GGCCCGTCAAAGTACGGGAA |
| | | CAGCTGTGCAAGCTGAAGGG |
| | | TGGGGTTGTAGTGGACGAGC |
| | | TTGGCTGCAGCCGCCAGCGA |
| | | GCCCCTTCCAGCAAACAGGT |
| | | GAATGGGGTACAGAAGCAAA |
| | | GGAGGCTGGCAGCAAACGCA |
| | | AGGGAACGGCGCAGGATGCA |
| | | CGGGCTGAACCACGCCTTCG |
| | | ACCAGCTGCGCAACGTTATC |
| | | CCGTCCTTCAACAACGACAA |
| | | GAAGCTGTCCAAATATGAGA |
| | | CCCTACAGATGGCCCAGATC |
| | | TACATCAACGCTCTGTCGGA |
| | | GTTGCTGCAGACTCCCAATG |
| | | TCGGAGAGCAACCGCCGCCG |
| | | CCCACAGCTTCCTGCAAAAA |
| | | TGACCACCATCACCTTCGCA |
| | | CCGCCTCCTCCTATGAAGGA |
| | | GGTGCGGGCGCCTCTGCGGT |
| | | AGCTGGGGCTCAGCCAGCCC |
| | | CGGGAGGGGGCCCGAGACCT |
| | | ACCCCGCCCGGGCCTTGCCG |
| | | GACTCGCTTCTCAGGCCCAG |
| | | CTTCCTCTGGGGGTTACTCG |
| | | GTGCAGCTGGACGCTTTGCA |
| | | CTTCCCAGCCTTCGAGGACA |
| | | GGGCCCTAACAGCGATGATG |
| | | GCACAGAAGGACCTGTCGCC |
| | | TTCGCTGCCCGGGGGCATCC |
| | | TGCAGCCTGTACAGGAGGAC |
| | | AACAGCAAAACATCTCCCAG |
| | | ATCCCACAGAAGTGACGGAG |
| | | AGTTTTCCCCCCACTCTCAT |
| | | TACAGTGACTCTGATGAGGC |
| | | CAGTTAGAAGCTTGGATCCA |
| | | ATCAACCTCTGGATTACAAA |
| | | ATTTGTGAAAGATTGACTGG |
| | | TATTCTTAACTATGTTGCTC |
| | | CTTTTACGCTATGTGGATAC |
| | | GCTGCTTTAATGCCTTTGTA |
| | | TCATGCTATTGCTTCCCGTA |
| | | TGGCTTTCATTTTCTCCTCC |
| | | TTGTATAAATCCTGGTTGCT |
| | | GTCTCTTTATGAGGAGTTGT |
| | | GGCCCGTTGTCAGGCAACGT |
| | | GGCGTGGTGTGCACTGTGTT |
| | | TGCTGACGCAACCCCCACTG |
| | | GTTGGGGCATTGCCACCACC |
| | | TGTCAGCTCCTTTCCGGGAC |
| | | TTTCGCTTTCCCCCTCCCTA |
| | | TTGCCACGGCGGAACTCATC |
| | | GCCGCCTGCCTTGCCCGCTG |
| | | CTGGACAGGGGCTCGGCTGT |
| | | TGGGCACTGACAATTCCGTG |
| | | GTGTTGTCGGGGAAATCATC |
| | | GTCCTTTCCTTGGCTGCTCG |
| | | CCTGTGTTGCCACCTGGATT |
| | | CTGCGCGGGACGTCCTTCTG |
| | | CTACGTCCCTTCGGCCCTCA |
| | | ATCCAGCGGACCTTCCTTCC |
| | | CGCGGCCTGCTGCCGGCTCT |
| | | GCGGCCTCTTCCGCGTCTTC |
| | | GAGATCTGCCTCGACTGTGC |
| | | CTTCTAGTTGCCAGCCATCT |
| | | GTTGTTTGCCCCTCCCCCGT |
| | | GCCTTCCTTGACCCTGGAAG |
| | | GTGCCACTCCCACTGTCCTT |
| | | TCCTAATAAAATGAGGAAAT |
| | | TGCATCGCATTGTCTGAGTA |
| | | GGTGTCATTCTATTCTGGGG |
| | | GGTGGGGTGGGGCAGGACAG |
| | | CAAGGGGGAGGATTGGGAAG |
| | | ACAATAGCAGGCATGCTGGG |
| | | GACTCGAGTTAAGGGCGAAT |
| | | TCCCGATAAGGATOTTCCTA |
| | | GAGCATGGCTACGTAGATAA |
| | | GTAGCATGGCGGGTTAATCA |
| | | TTAACTACAAGGAACCCCTA |
| | | GTGATGGAGTTGGCCACTCC |
| | | CTCTCTGCGCGCTCGCTCGC |
| | | TCACTGAGGCCGGGCGACCA |
| | | AAGGTCGCCCGACGCCCGGG |
| | | CTTTGCCCGGGCGGCCTCAG |
| | | TGAGCGAGCGAGCGCGCAGC |
| | | CTTAATTAACCTAATTCACT |
| | | GGCCGTCGTTTTACAACGTC |
| | | GTGACTGGGAAAACCCTGGC |
| | | GTTACCCAACTTAATCGCCT |
| | | TGCAGCACATCCCCCTTTCG |
| | | CCAGCTGGCGTAATAGCGAA |
| | | GAGGCCCGCACCGATCGCCC |
| | | TTCCCAACAGTTGCGCAGCC |
| | | TGAATGGCGAATGGGACGCG |
| | | CCCTGTAGCGGCGCATTAAG |
| | | CGCGGCGGGTGTGGTGGTTA |
| | | CGCGCAGCGTGACCGCTACA |
| | | CTTGCCAGCGCCCTAGCGCC |
| | | CGCTCCTTTCGCTTTCTTCC |
| | | CTTCCTTTCTCGCCACGTTC |
| | | GCCGGCTTTCCCCGTCAAGC |
| | | TCTAAATCGGGGGCTCCCTT |
| | | TAGGGTTCCGATTTAGTGCT |
| | | TTACGGCACCTCGACCCCAA |
| | | AAAACTTGATTAGGGTGATG |
| | | GTTCACGTAGTGGGCCATCG |
| | | CCCTGATAGACGGTTTTTCG |
| | | CCCTTTGACGTTGGAGTCCA |
| | | CGTTCTTTAATAGTGGACTC |
| | | TTGTTCCAAACTGGAACAAC |
| | | ACTCAACCCTATCTCGGTCT |
| | | ATTCTTTTGATTTATAAGGG |
| | | ATTTTGCCGATTTCGGCCTA |
| | | TTGGTTAAAAAATGAGCTGA |
| | | TTTAACAAAAATTTAACGCG |
| | | AATTTTAACAAAATATTAAC |
| | | GCTTACAATTTAGGTGGCAC |
| | | TTTTCGGGGAAATGTGCGCG |
| | | GAACCCCTATTTGTTTATTT |
| | | TTCTAAATACATTCAAATAT |
| | | GTATCCGCTCATGAGACAAT |
| | | AACCCTGATAAATGCTTCAA |
| | | TAATATTGAAAAAGGAAGAG |
| | | TATGAGCCATATTCAACGGG |
| | | AAACGTCGAGGCCGCGATTA |
| | | AATTCCAACATGGATGCTGA |
| | | TTTATATGGGTATAAATGGG |
| | | CTCGCGATAATGTCGGGCAA |
| | | TCAGGTGCGACAATCTATCG |
| | | CTTGTATGGGAAGCCCGATG |
| | | CGCCAGAGTTGTTTCTGAAA |
| | | CATGGCAAAGGTAGCGTTGC |
| | | CAATGATGTTACAGATGAGA |
| | | TGGTCAGACTAAACTGGCTG |
| | | ACGGAATTTATGCCTCTTCC |
| | | GACCATCAAGCATTTTATCC |
| | | GTACTCCTGATGATGCATGG |
| | | TTACTCACCACTGCGATCCC |
| | | CGGAAAAACAGCATTCCAGG |
| | | TATTAGAAGAATATCCTGAT |
| | | TCAGGTGAAAATATTGTTGA |
| | | TGCGCTGGCAGTGTTCCTGC |
| | | GCCGGTTGCATTCGATTCCT |
| | | GTTTGTAATTGTCCTTTTAA |
| | | CAGCGATCGCGTATTTCGTC |
| | | TTGCTCAGGCGCAATCACGA |
| | | ATGAATAACGGTTTGGTTGA |
| | | TGCGAGTGATTTTGATGACG |
| | | AGCGTAATGGCTGGCCTGTT |
| | | GAACAAGTCTGGAAAGAAAT |
| | | GCATAAACTTTTGCCATTCT |
| | | CACCGGATTCAGTCGTCACT |
| | | CATGGTGATTTCTCACTTGA |
| | | TAACCTTATTTTTGACGAGG |
| | | GGAAATTAATAGGTTGTATT |
| | | GATGTTGGACGAGTCGGAAT |
| | | CGCAGACCGATACCAGGATC |
| | | TTGCCATCCTATGGAACTGC |
| | | CTCGGTGAGTTTTCTCCTTC |
| | | ATTACAGAAACGGCTTTTTC |
| | | AAAAATATGGTATTGATAAT |
| | | CCTGATATGAATAAATTGCA |
| | | GTTTCATTTGATGCTCGATG |
| | | AGTTTTTCTAACTGTCAGAC |
| | | CAAGTTTACTCATATATACT |
| | | TTAGATTGATTTAAAACTTC |
| | | ATTTTTAATTTAAAAGGATC |
| | | TAGGTGAAGATCCTTTTTGA |
| | | TAATCTCATGACCAAAATCC |
| | | CTTAACGTGAGTTTTCGTTC |
| | | CACTGAGCGTCAGACCCCGT |
| | | AGAAAAGATCAAAGGATCTT |
| | | CTTGAGATCCTTTTTTTCTG |
| | | CGCGTAATCTGCTGCTTGCA |
| | | AACAAAAAAACCACCGCTAC |
| | | CAGCGGTGGTTTGTTTGCCG |
| | | GATCAAGAGCTACCAACTCT |
| | | TTTTCCGAAGGTAACTGGCT |
| | | TCAGCAGAGCGCAGATACCA |
| | | AATACTGTTCTTCTAGTGTA |
| | | GCCGTAGTTAGGCCACCACT |
| | | TCAAGAACTCTGTAGCACCG |
| | | CCTACATACCTCGCTCTGCT |
| | | AATCCTGTTACCAGTGGCTG |
| | | CTGCCAGTGGCGATAAGTCG |
| | | TGTCTTACCGGGTTGGACTC |
| | | AAGACGATAGTTACCGGATA |
| | | AGGCGCAGCGGTCGGGCTGA |
| | | ACGGGGGGTTCGTGCACACA |
| | | GCCCAGCTTGGAGCGAACGA |
| | | CCTACACCGAACTGAGATAC |
| | | CTACAGCGTGAGCTATGAGA |
| | | AAGCGCCACGCTTCCCGAAG |
| | | GGAGAAAGGCGGACAGGTAT |
| | | CCGGTAAGCGGCAGGGTCGG |
| | | AACAGGAGAGCGCACGAGGG |
| | | AGCTTCCAGGGGGAAACGCC |
| | | TGGTATCTTTATAGTCCTGT |
| | | CGGGTTTCGCCACCTCTGAC |
| | | TTGAGCGTCGATTTTTGTGA |
| | | TGCTCGTCAGGGGGGCGGAG |
| | | CCTATGGAAAAACGCCAGCA |
| | | ACGCGGCCTTTTTACGGTTC |
| | | CTGGCCTTTTGCTGGCCTTT |
| | | TGCTCACATGTTCTTTCCTG |
| | | CGTTATCCCCTGATTCTGTG |
| | | GATAACCGTATTACCGCCTT |
| | | TGAGTGAGCTGATACCGCTC |
| | | GCCGCAGCCGAACGACCGAG |
| | | CGCAGCGAGTCAGTGAGCGA |
| | | GGAAGCGGAAGAGCGCCCAA |
| | | TACGCAAACCGCCTCTCCCC |
| | | GCGCGTTGGCCGATTCATTA |
| | | ATGCAGCTGGCACGACAGGT |
| | | TTCCCGACTGGAAAGCGGGC |
| | | AGTGAGCGCAACGCAATTAA |
| | | TGTGAGTTAGCTCACTCATT |
| | | AGGCACCCCAGGCTTTACAC |
| | | TTTATGCTTCCGGCTCGTAT |
| | | GTTGTGTGGAATTGTGAGCG |
| | | GATAACAATTTCACACAGGA |
| | | AACAGCTATGACCATGATTA |
| | | CGCCAGATTTAATTAAGGCC |
| | | TTAATTAGG |

### Methods for the Delivery of Exogenous Nucleic Acids to Target Cells

Techniques that can be used to introduce a transgene, such as a transgene operably linked to an SLC6A14 promoter described herein, into a target cell (e.g., a mammalian cell) are well known in the art. For instance, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for instance, by direct fusion of the liposome with the cell membrane or by endocytosis of the complex. Lipofection is described in detail, for instance, in U.S. Pat. No. 7,442,386, the disclosure of which is incorporated herein by reference. Similar techniques that exploit ionic interactions with the cell membrane to provoke the uptake of foreign nucleic acids include contacting a cell with a cationic polymer-nucleic acid complex. Exemplary cationic molecules that associate with polynucleotides so as to impart a positive charge favorable for interaction with the cell membrane include activated dendrimers (described, e.g., in Dennig, Topics in Current Chemistry 228:227 (2003), the disclosure of which is incorporated herein by reference) polyethyleneimine, and diethylaminoethyl (DEAE)-dextran, the use of which as a transfection agent is described in detail, for instance, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for instance, in US 2010/0227406, the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107: 1870 (2010), the disclosure of which is incorporated herein by reference.

Magnetofection can also be used to deliver nucleic acids to target cells. The magnetofection principle is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, RNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane to permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For instance, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Vectors for Delivery of Exogenous Nucleic Acids to Target Cells

In addition to achieving high rates of transcription and translation, stable expression of an exogenous gene in a mammalian cell can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Examples of expression vectors are described in, e.g., Gellissen, Production of Recombinant Proteins: Novel Microbial and Eukaryotic Expression Systems (John Wiley & Sons, Marblehead, M A, 2006). Expression vectors for use in the compositions and methods described herein contain an SLC6A14 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1) operably linked to a polynucleotide sequence that encodes a protein of interest, as well as, e.g., additional sequence elements used for the expression of these agents and/or the integration of these polynucleotide sequences into the genome of a mammalian cell. Vectors that can contain an SLC6A14 promoter operably linked to a transgene encoding a protein of interest include plasmids (e.g., circular DNA molecules that can autonomously replicate inside a cell), cosmids (e.g., pWE or sCos vectors), artificial chromosomes (e.g., a human artificial chromosome (HAC), a yeast artificial chromosome (YAC), a bacterial artificial chromosome (BAC), or a P1-derived artificial chromosome (PAC)), and viral vectors. Certain vectors that can be used for the expression of a protein of interest include plasmids that contain regulatory sequences, such as enhancer regions, which direct gene transcription. Other useful vectors for expression of a protein of interest contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### Viral Vectors for Nucleic Acid Delivery

Viral genomes provide a rich source of vectors that can be used for the efficient delivery of a gene of interest into the genome of a target cell (e.g., a mammalian cell, such as a human cell). Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, β-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, 1996)). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, U.S. Pat. No. 5,801,030, the disclosure of which is incorporated herein by reference as it pertains to viral vectors for use in gene therapy.

### AAV Vectors for Nucleic Acid Delivery

In some embodiments, polynucleotides of the compositions and methods described herein are incorporated into rAAV vectors and/or virions in order to facilitate their introduction into a cell (e.g., a VSC). rAAV vectors useful in the compositions and methods described herein are recombinant nucleic acid constructs that include (1) an SLC6A14 promoter described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1), (2) a heterologous sequence to be expressed, and (3) viral sequences that facilitate stability and expression of the heterologous genes. The viral sequences may include those sequences of AAV that are required in cis for replication and packaging (e.g., functional ITRs) of the DNA into a virion. In typical applications, the transgene encodes a protein that can promote or increase vestibular hair cell development, vestibular hair cell fate specification, vestibular hair cell regeneration, vestibular hair cell and/or VSC proliferation, vestibular hair cell innervation, or vestibular hair cell maturation, or a wild-type form of a vestibular hair cell protein that is mutated in subjects with forms of hereditary vestibular dysfunction that may be useful for improving vestibular function in subjects carrying a mutation associated with vestibular dysfunction (e.g., dizziness, vertigo, imbalance, bilateral vestibulopathy, bilateral vestibular hypofunction, oscillopsia, or a balance disorder). Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. For use in the methods and compositions described herein, the ITRs can be AAV2 ITRs. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The polynucleotides and vectors described herein (e.g., an SLC6A14 promoter operably linked to a transgene encoding a protein of interest) can be incorporated into a rAAV virion in order to facilitate introduction of the polynucleotide or vector into a cell (e.g., a VSC). The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10, 11, rh10, rh39, rh43, rh74, Anc80, Anc80L65, DJ/8, DJ/9, 7m8, PHP.B, PHP.eB, and PHP.S. For targeting VSCs, AAV1, AAV2, AAV2quad(Y-F), AAV3, AAV4, AAV5, AAV6, AAV7, AAV8, AAV9, Anc80, Anc80L65, 7m8, PHP.B, PHP.eB, or PHP.S serotypes may be particularly useful. Serotypes evolved for transduction of the retina may also be used in the methods and compositions described herein. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV2quad(Y-F), AAV3, AAV4, AAV5, AAV6, AAV7, AAV8, etc.). Techniques involving the construction and use of pseudotyped rAAV virions are known in the art and are described, for instance, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

In some embodiments, the nucleic acid vector (e.g., an AAV vector) includes an SLC6A14 promoter described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1) operably linked to a polynucleotide sequence encoding human Atoh1 (human ATOH1 protein=RefSeq Accession No. NP_005163 (SEQ ID NO: 4); mRNA sequence=RefSeq Accession No. NM_005172). In some embodiments, the SLC6A14 promoter is the SLC6A14 promoter of SEQ ID NO: 1 (also represented by nucleotides 219-1977 of SEQ ID NO: 10) and it is operably linked to a polynucleotide sequence encoding human Atoh1. In some embodiments, the polynucleotide sequence encoding human Atoh1 is SEQ ID NO: 5. In some embodiments, the polynucleotide sequence that encodes human Atoh1 is any polynucleotide sequence that, by redundancy of the genetic code, encodes SEQ ID NO: 4. The polynucleotide sequence that encodes human Atoh1 can be partially or fully codon-optimized for expression. In some embodiments, the vector includes, in 5′ to 3′ order, a first inverted terminal repeat; an SLC6A14 promoter of SEQ ID NO: 1; a polynucleotide sequence encoding human Atoh1 operably linked to the SLC6A14 promoter; a polyadenylation sequence; and a second inverted terminal repeat. In some embodiments, the nucleic acid vector includes, in 5′ to 3′ order, a first inverted terminal repeat; an SLC6A14 promoter of SEQ ID NO: 1; a polynucleotide sequence encoding human Atoh1 operably linked to the SLC6A14 promoter; a Woodchuck Posttranscriptional Regulatory Element (WPRE); a polyadenylation sequence; and a second inverted terminal repeat. In some embodiments, the WPRE has the sequence of SEQ ID NO: 8 or SEQ ID NO: 9. In some embodiments, the WPRE has the sequence of SEQ ID NO: 8. In some embodiments, the WPRE has the sequence of nucleotides 3064-3611 of SEQ ID NO: 10. In some embodiments, the polyadenylation sequence has the sequence of nucleotides 3624-3831 of SEQ ID NO: 10. In certain embodiments, the nucleic acid vector includes nucleotides 219-3831 of SEQ ID NO: 10, flanked by inverted terminal repeats. In some embodiments, the inverted terminal repeats are AAV2 inverted terminal repeats. In some embodiments, the inverted terminal repeats are any variant of AAV2 inverted terminal repeats that can be encapsidated by a plasmid that carries the AAV2 Rep gene. In particular embodiments, the nucleic acid vector includes nucleotides 219-3831 of SEQ ID NO: 10, flanked by inverted terminal repeats, in which the 5′ inverted terminal repeat has at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to nucleotides 1-130 of SEQ ID NO: 10; and in which the 3′ inverted terminal repeat has at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to nucleotides 3919-4048 of SEQ ID NO: 10. In some embodiments, the nucleic acid vector is a viral vector. In some embodiments, the viral vector is an AAV vector. In some embodiments, the AAV vector has an AAV8 capsid.

In some embodiments, the nucleic acid vector (e.g., an AAV vector) includes an SLC6A14 promoter described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1) operably linked to a polynucleotide sequence encoding murine Atoh1 (murine ATOH1 protein=UniProt P48985 (SEQ ID NO: 6); mRNA sequence=RefSeq Accession No. NM_007500.5). In some embodiments, the SLC6A14 promoter is the SLC6A14 promoter of SEQ ID NO: 1 (also represented by nucleotides 219-1977 of SEQ ID NO: 11) and it is operably linked to a polynucleotide sequence encoding murine Atoh1. In some embodiments, the polynucleotide sequence encoding murine Atoh1 is SEQ ID NO: 7. In some embodiments, the polynucleotide sequence that encodes murine Atoh1 is any polynucleotide sequence that, by redundancy of the genetic code, encodes SEQ ID NO: 6. The polynucleotide sequence that encodes murine Atoh1 can be partially or fully codon-optimized for expression. In some embodiments, the vector includes, in 5′ to 3′ order, a first inverted terminal repeat; an SLC6A14 promoter of SEQ ID NO: 1; a polynucleotide sequence encoding murine Atoh1 operably linked to the SLC6A14 promoter; a polyadenylation sequence; and a second inverted terminal repeat. In some embodiments, the nucleic acid vector includes, in 5′ to 3′ order, a first inverted terminal repeat; an SLC6A14 promoter of SEQ ID NO: 1; a polynucleotide sequence encoding murine Atoh1 operably linked to the SLC6A14 promoter; a Woodchuck Posttranscriptional Regulatory Element (WPRE); a polyadenylation sequence; and a second inverted terminal repeat. In some embodiments, the WPRE has the sequence of SEQ ID NO: 8 or SEQ ID NO: 9. In some embodiments, the WPRE has the sequence of SEQ ID NO: 8. In some embodiments, the WPRE has the sequence of nucleotides 3055-3602 of SEQ ID NO: 11. In some embodiments, the polyadenylation sequence has the sequence of nucleotides 3615-3822 of SEQ ID NO: 11. In certain embodiments, the nucleic acid vector includes nucleotides 219-3822 of SEQ ID NO: 11, flanked by inverted terminal repeats. In some embodiments, the inverted terminal repeats are AAV2 inverted terminal repeats. In some embodiments, the inverted terminal repeats are any variant of AAV2 inverted terminal repeats that can be encapsidated by a plasmid that carries the AAV2 Rep gene. In particular embodiments, the nucleic acid vector includes nucleotides 219-3822 of SEQ ID NO: 11, flanked by inverted terminal repeats, in which the 5′ inverted terminal repeat has at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to nucleotides 1-130 of SEQ ID NO: 11; and in which the 3′ inverted terminal repeat has at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to nucleotides 3910-4039 of SEQ ID NO: 11. In some embodiments, the nucleic acid vector is a viral vector. In some embodiments, the viral vector is an AAV vector. In some embodiments, the AAV vector has an AAV8 capsid.

It should be understood by those of ordinary skill in the art that the creation of a viral vector of the invention typically requires the use of a plasmid of the invention together with additional plasmids that provide required elements for proper viral packaging and viability (e.g., for AAV, plasmids providing the appropriate AAV rep gene, cap gene and other genes (e.g., E2A and E4)). The combination of those plasmids in a producer cell line produces the viral vector. However, it will be understood by those of skill in the art, that for any given pair of inverted terminal repeat sequences in a transfer plasmid of the invention that is used to create the viral vector, the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom.

### Pharmaceutical Compositions

The polynucleotides described herein (e.g., an SLC6A14 promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1) may be operably linked to a transgene (e.g., a transgene encoding a protein of interest, an shRNA, an ASO, or a nuclease (e.g., Cas9, TALEN, ZFN, or gRNA), or a transgene that can be transcribed to produce a microRNA) and incorporated into a vehicle for administration into a patient, such as a human patient suffering from vestibular dysfunction. Pharmaceutical compositions containing vectors, such as viral vectors, that contain a polynucleotide described herein operably linked to a transgene can be prepared using methods known in the art. For example, such compositions can be prepared using, e.g., physiologically acceptable carriers, excipients, or stabilizers (Remington: The Science and Practice of Pharmacology 22nd edition, Allen, L. Ed. (2013); incorporated herein by reference), and in a desired form, e.g., in the form of lyophilized formulations or aqueous solutions.

Mixtures of nucleic acid vectors (e.g., viral vectors) containing an SLC6A14 promoter described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1) operably linked to a transgene may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. For local administration to the middle or inner ear, the composition may be formulated to contain a synthetic perilymph solution. An exemplary synthetic perilymph solution includes 20-200 mM NaCl, 1-5 mM KCl, 0.1-10 mM CaCl₂), 1-10 mM glucose, and 2-50 mM HEPEs, with a pH between about 6 and 9 and an osmolality of about 300 mOsm/kg. The person responsible for administration will, in any event, determine the appropriate dose for the individual subject. Moreover, for human administration, preparations may meet sterility, pyrogenicity, general safety, and purity standards as required by FDA Office of Biologics standards.

### Methods of Treatment

The compositions described herein may be administered to a subject having or at risk of developing vestibular dysfunction by a variety of routes, such as local administration to the middle or inner ear (e.g., administration into the perilymph or endolymph, such as through the oval window, round window, or semicircular canal (e.g., the horizontal canal), or by transtympanic or intratympanic injection, e.g., administration to a vestibular supporting cell or hair cell), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. The most suitable route for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bi-monthly, monthly, or bi-weekly).

Subjects that may be treated as described herein are subjects having or at risk of developing vestibular dysfunction. The compositions and methods described herein can be used to treat subjects having or at risk of developing damage to vestibular hair cells (e.g., damage related to disease or infection, head trauma, ototoxic drugs (e.g., aminoglycosides), or aging), subjects having or at risk of developing vestibular dysfunction (e.g., dizziness, vertigo, imbalance, bilateral vestibulopathy (also called bilateral vestibular hypofunction), oscillopsia, or a balance disorder), subjects carrying a genetic mutation associated with vestibular dysfunction, or subjects with a family history of hereditary vestibular dysfunction. In some embodiments, the disease associated with damage to or loss of hair cells (e.g., vestibular hair cells) is an autoimmune disease or condition in which an autoimmune response contributes to hair cell damage or death. Autoimmune diseases linked to vestibular dysfunction include autoimmune inner ear disease (AIED), polyarteritis nodosa (PAN), Cogan's syndrome, relapsing polychondritis, systemic lupus erythematosus (SLE), Wegener's granulomatosis, Sjögren's syndrome, and Behcet's disease. Some infectious conditions, such as Lyme disease and syphilis can also cause vestibular dysfunction (e.g., by triggering autoantibody production). Viral infections, such as rubella, cytomegalovirus (CMV), lymphocytic choriomeningitis virus (LCMV), HSV types 1&2, West Nile virus (WNV), human immunodeficiency virus (HIV) varicella zoster virus (VZV), measles, and mumps, can also cause vestibular dysfunction. In some embodiments, the subject has vestibular dysfunction that is associated with or results from loss of hair cells (e.g., vestibular hair cells). In some embodiments, compositions and methods described herein can be used to treat a subject having or at risk of developing oscillopsia. In some embodiments, compositions and methods described herein can be used to treat a subject having or at risk of developing bilateral vestibulopathy. In some embodiments, the compositions and methods described herein can be used to treat a subject having or at risk of developing a balance disorder (e.g., imbalance). The methods described herein may include a step of screening a subject for one or more mutations in genes known to be associated with vestibular dysfunction prior to treatment with or administration of the compositions described herein. A subject can be screened for a genetic mutation using standard methods known to those of skill in the art (e.g., genetic testing). The methods described herein may also include a step of assessing vestibular function in a subject prior to treatment with or administration of the compositions described herein. Vestibular function may be assessed using standard tests, such as eye movement testing (e.g., electronystagmogram (ENG) or videonystagmogram (VNG)), tests of the vestibulo-ocular reflex (VOR) (e.g., the head impulse test (Halmagyi-Curthoys test), which can be performed at the bedside or using a video-head impulse test (VHIT), or the caloric reflex test), posturography, rotary-chair testing, ECOG, vestibular evoked myogenic potentials (VEMP), and specialized clinical balance tests, such as those described in Mancini and Horak, Eur J Phys Rehabil Med, 46:239 (2010). These tests can also be used to assess vestibular function in a subject after treatment with or administration of the compositions described herein. The compositions and methods described herein may also be administered as a preventative treatment to patients at risk of developing vestibular dysfunction, e.g., patients who have a family history of vestibular dysfunction (e.g., inherited vestibular dysfunction), patients carrying a genetic mutation associated with vestibular dysfunction who do not yet exhibit symptoms of vestibular dysfunction, or patients exposed to risk factors for acquired vestibular dysfunction (e.g., disease or infection, head trauma, ototoxic drugs, or aging). The compositions and methods described herein can also be used to treat a subject with idiopathic vestibular dysfunction.

The compositions and methods described herein can be used to induce or increase hair cell regeneration in a subject (e.g., vestibular hair cell regeneration), and/or to induce or increase proliferation of vestibular hair cells and/or VSCs. Subjects that may benefit from compositions that promote or induce vestibular hair cell regeneration, vestibular hair cell innervation, and/or vestibular hair cell and/or VSC proliferation include subjects having or at risk of developing vestibular dysfunction as a result of loss of hair cells (e.g., loss of vestibular hair cells related to trauma (e.g., head trauma), disease or infection, ototoxic drugs, or aging), and subjects with abnormal vestibular hair cells (e.g., vestibular hair cells that do not function properly compared to normal vestibular hair cells), damaged vestibular hair cells (e.g., vestibular hair cell damage related to trauma (e.g., head trauma), disease or infection, ototoxic drugs, or aging), or reduced vestibular hair cell numbers due to genetic mutations or congenital abnormalities. The compositions and methods described herein can also be used to promote or increase vestibular hair cell maturation, which can lead to improved vestibular function. In some embodiments, the compositions and methods described herein promote or increase the maturation of regenerated vestibular hair cells (e.g., promote or increase the maturation of vestibular hair cells formed in response to expression of a composition described herein, such as a composition containing an SLC6A14 promoter operably linked to a transgene, in VSCs). The compositions and methods described herein can also promote or increase VSC and/or vestibular hair cell survival and/or improve VSC function.

The compositions and methods described herein can also be used to prevent or reduce vestibular dysfunction caused by ototoxic drug-induced hair cell damage or death (e.g., vestibular hair cell damage or death) in subjects who have been treated with ototoxic drugs, or who are currently undergoing or soon to begin treatment with ototoxic drugs. Ototoxic drugs are toxic to the cells of the inner ear, and can cause vestibular dysfunction (e.g., vertigo, dizziness, imbalance, bilateral vestibulopathy, or oscillopsia). Drugs that have been found to be ototoxic include aminoglycoside antibiotics (e.g., gentamycin, neomycin, streptomycin, tobramycin, kanamycin, vancomycin, and amikacin), viomycin, antineoplastic drugs (e.g., platinum-containing chemotherapeutic agents, such as cisplatin, carboplatin, and oxaliplatin), loop diuretics (e.g., ethacrynic acid and furosemide), salicylates (e.g., aspirin, particularly at high doses), and quinine. In some embodiments, the methods and compositions described herein can be used to treat bilateral vestibulopathy. In some embodiments, the methods and compositions described herein can be used to treat bilateral vestibulopathy or oscillopsia due to aminoglycoside ototoxicity (e.g., the methods and compositions described herein can be used to reduce aminoglycoside-induced vestibular hair cell damage or death, or to promote or increase hair cell regeneration and/or hair cell or VSC proliferation in a subject with aminoglycoside-induced bilateral vestibulopathy or oscillopsia).

The transgene operably linked to an SLC6A14 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1) for treatment of a subject as described herein can be a transgene that encodes a protein expressed in healthy VSCs (e.g., a protein that plays a role in vestibular hair cell development, vestibular hair cell fate specification, vestibular hair cell regeneration, vestibular hair cell and/or VSC proliferation, vestibular hair cell maturation, or vestibular hair cell innervation, or a protein that is deficient in a subject with vestibular dysfunction), another protein of interest (e.g., a therapeutic protein or a reporter protein, such as a fluorescent protein, lacZ, or luciferase), an shRNA, an ASO, a nuclease, or a microRNA. The transgene may be selected based on the cause of the subject's vestibular dysfunction (e.g., if the subject's vestibular dysfunction is associated with a particular genetic mutation, the transgene can be a wild-type form of the gene that is mutated in the subject, or if the subject has vestibular dysfunction associated with loss of hair cells, the transgene can encode a protein that promotes vestibular hair cell regeneration, vestibular hair cell innervation, or vestibular hair cell and/or VSC proliferation), the severity of the subject's vestibular dysfunction, the health of the subject's hair cells, the subject's age, the subject's family history of vestibular dysfunction, or other factors. The proteins that may be expressed by a transgene operably linked an SLC6A14 promoter for treatment of a subject as described herein include Sox9, Sall2, Camta1, Hey2, Gata2, Hey1, Lass2, Sox10, Gata3, Cux1, Nr2f1, Hes1, Rorb, Jun, Zfp667, Lhx3, Nhlh1, Mxd4, Zmiz1, Myt1, Stat3, Barhl1, Tox, Prox1, Nfia, Thrb, Mycl1, Kdm5a, Creb314, Etv1, Peg3, Bach2, Isl1, Zbtb38, Lbh, Tub, Hmg20, Rest, Zfp827, Aff3, Pknox2, Arid3b, Mixip, Zfp532, Ikzf2, Sall1, Six2, Sall3, Lin28b, Rfx7, Bdnf, Gfi1, Pou4f3, Myc, Ctnnb1, Sox2, Sox4, Sox11, Tead2, Atoh1, and an Atoh1 variant containing substitutions at amino acids 328, 331, and/or 334 (e.g., S328A, S331A, S334A, S328A/S331A, S328A/S334A, S331A/S334A, and 328A/S331A/S334).

Treatment may include administration of a composition containing a nucleic acid vector (e.g., an AAV viral vector) containing an SLC6A14 promoter described herein (e.g., SEQ ID NO: 1) in various unit doses. Each unit dose will ordinarily contain a predetermined quantity of the therapeutic composition. The quantity to be administered, and the particular route of administration and formulation, are within the skill of those in the clinical arts. A unit dose need not be administered as a single injection but may include continuous infusion over a set period of time. Dosing may be performed using a syringe pump to control infusion rate in order to minimize damage to the inner ear (e.g., the vestibular labyrinth). In cases in which the nucleic acid vectors are AAV vectors (e.g., AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S vectors), the viral vectors may be administered to the patient at a dose of, for example, from about 1×10⁹ vector genomes (VG)/mL to about 1×10¹⁶ VG/mL (e.g., 1×10^{9 VG/mL,} 2×10⁹ VG/mL, 3×10⁹ VG/mL, 4×10⁹ VG/mL, 5×10⁹ VG/mL, 6×10⁹ VG/mL, 7×10⁹ VG/mL, 8×10⁹ VG/mL, 9×10⁹ VG/mL, 1×10¹⁰ VG/mL, 2×10¹⁰ VG/mL, 3×10¹⁰ VG/mL, 4×10¹⁰ VG/mL, 5×10¹⁰ VG/mL, 6×10¹⁰ VG/mL, 7×10¹⁰ VG/mL, 8×10¹⁰ VG/mL, 9×10¹⁰ VG/mL, 1×10¹¹ VG/mL, 2×10¹¹ VG/mL, 3×10¹¹ VG/mL, 4×10¹¹ VG/mL, 5×10¹¹ VG/mL, 6×10¹¹ VG/mL, 7×10¹¹ VG/mL, 8×10¹¹ VG/mL, 9×10¹¹ VG/mL, 1×10¹² VG/mL, 2×10¹² VG/mL, 3×10¹² VG/mL, 4×10¹² VG/mL, 5×10¹² VG/mL, 6×10¹² VG/mL, 7×10¹² VG/mL, 8×10¹² VG/mL, 9×10¹² VG/mL, 1×10¹³ VG/mL, 2×10¹³ VG/mL, 3×10¹³ VG/mL, 4×10¹³ VG/mL, 5×10¹³ VG/mL, 6×10¹³ VG/mL, 7×10¹³ VG/mL, 8×10¹³ VG/mL, 9×10¹³ VG/mL, 1×10¹⁴ VG/mL, 2×10¹⁴ VG/mL, 3×10¹⁴ VG/mL, 4×10¹⁴ VG/mL, 5×10¹⁴ VG/mL, 6×10¹⁴ VG/mL, 7×10¹⁴ VG/mL, 8×10¹⁴ VG/mL, 9×10¹⁴ VG/mL, 1×10¹⁵ VG/mL, 2×10¹⁵ VG/mL, 3×10¹⁵ VG/mL, 4×10¹⁵ VG/mL, 5×10¹⁵ VG/mL, 6×10¹⁵ VG/mL, 7×10¹⁵ VG/mL, 8×10¹⁵ VG/mL, 9×10¹⁵ VG/mL, or 1×10¹⁶ VG/mL) in a volume of 1 μL to 200 μL (e.g., 1, 2, 3, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μL). The AAV vectors may be administered to the subject at a dose of about 1×10⁷ VG/ear to about 2×10¹⁵ VG/ear (e.g., 1×10⁷ VG/ear, 2×10⁷ VG/ear, 3×10⁷ VG/ear, 4×10⁷ VG/ear, 5×10⁷ VG/ear, 6×10⁷ VG/ear, 7×10⁷ VG/ear, 8×10⁷ VG/ear, 9×10⁷ VG/ear, 1×10⁸ VG/ear, 2×10⁸ VG/ear, 3×10⁸ VG/ear, 4×10⁸ VG/ear, 5×10⁸ VG/ear, 6×10⁸ VG/ear, 7×10⁸ VG/ear, 8×10⁸ VG/ear, 9×10⁸ VG/ear, 1×10⁹ VG/ear, 2×10⁹ VG/ear, 3×10⁹ VG/ear, 4×10⁹ VG/ear, 5×10⁹ VG/ear, 6×10⁹ VG/ear, 7×10⁹ VG/ear, 8×10⁹ VG/ear, 9×10⁹ VG/ear, 1×10¹⁰ VG/ear, 2×10¹⁰ VG/ear, 3×10¹⁰ VG/ear, 4×10¹⁰ VG/ear, 5×10¹⁰ VG/ear, 6×10¹⁰ VG/ear, 7×10¹⁰ VG/ear, 8×10¹⁰ VG/ear, 9×10¹⁰ VG/ear, 1×10¹¹ VG/ear, 2×10¹¹ VG/ear, 3×10¹¹ VG/ear, 4×10¹¹ VG/ear, 5×10¹¹ VG/ear, 6×10¹¹ VG/ear, 7×10¹¹ VG/ear, 8×10¹¹ VG/ear, 9×10¹¹ VG/ear, 1×10¹² VG/ear, 2×10¹² VG/ear, 3×10¹² VG/ear, 4×10¹² VG/ear, 5×10¹² VG/ear, 6×10¹² VG/ear, 7×10¹² VG/ear, 8×10¹² VG/ear, 9×10¹² VG/ear, 1×10¹³ VG/ear, 2×10¹³ VG/ear, 3×10¹³ VG/ear, 4×10¹³ VG/ear, 5×10¹³ VG/ear, 6×10¹³ VG/ear, 7×10¹³ VG/ear, 8×10¹³ VG/ear, 9×10¹³ VG/ear, 1×10¹⁴ VG/ear, 2×10¹⁴ VG/ear, 3×10¹⁴ VG/ear, 4×10¹⁴ VG/ear, 5×10¹⁴ VG/ear, 6×10¹⁴ VG/ear, 7×10¹⁴ VG/ear, 8×10¹⁴ VG/ear, 9×10¹⁴ VG/ear, 1×10¹⁵ VG/ear, or 2×10¹⁵ VG/ear).

The compositions described herein are administered in an amount sufficient to improve vestibular function (e.g., improve balance or reduce dizziness or vertigo), treat bilateral vestibulopathy, treat oscillopsia, treat a balance disorder, increase expression of a protein encoded by a transgene operably linked to an SLC6A14 promoter, increase function of a protein encoded by a transgene operably linked to an SLC6A14 promoter, promote or increase hair cell development, increase hair cell numbers (e.g., promote or induce hair cell regeneration or proliferation), increase or induce hair cell maturation (e.g., the maturation of regenerated hair cells), improve hair cell function, improve VSC function, promote or increase VSC and/or vestibular hair cell survival, and/or promote or increase VSC proliferation. Vestibular function may be evaluated using standard tests for balance and vertigo (e.g., eye movement testing (e.g., ENG or VNG), VOR testing (e.g., head impulse testing (Halmagyi-Curthoys testing, e.g., VHIT), or caloric reflex testing), posturography, rotary-chair testing, ECOG, VEMP, and specialized clinical balance tests) and may be improved by 5% or more (e.g., 5%, 10%, 15%, 20%, 30%, 40%, 50%, 60%, 70%, 80%, 90%, 100%, 125%, 150%, 200% or more) compared to measurements obtained prior to treatment. The compositions described herein may also be administered in an amount sufficient to slow or prevent the development or progression of vestibular dysfunction (e.g., in subjects who carry a genetic mutation associated with vestibular dysfunction, who have a family history of vestibular dysfunction (e.g., hereditary vestibular dysfunction), or who have been exposed to risk factors associated with vestibular dysfunction (e.g., ototoxic drugs, head trauma, or disease or infection) but who do not exhibit vestibular dysfunction (e.g., vertigo, dizziness, or imbalance), or in subjects exhibiting mild to moderate vestibular dysfunction). Expression of the protein encoded by the transgene operably linked to an SLC6A14 promoter in the nucleic acid vector administered to the subject may be evaluated using immunohistochemistry, Western blot analysis, quantitative real-time PCR, or other methods known in the art for detection protein or mRNA, and may be increased by 5% or more (e.g., 5%, 10%, 15%, 20%, 30%, 40%, 50%, 60%, 70%, 80%, 90%, 100%, 125%, 150%, 200% or more) compared to expression prior to administration of a composition described herein. Hair cell numbers, hair cell function, hair cell maturation, hair cell regeneration, or function of the protein encoded by the nucleic acid vector administered to the subject may be evaluated indirectly based on tests of vestibular function, and may be increased by 5% or more (e.g., 5%, 10%, 15%, 20%, 30%, 40%, 50%, 60%, 70%, 80%, 90%, 100%, 125%, 150%, 200% or more) compared to hair cell numbers, hair cell function, hair cell maturation, hair cell regeneration, or function of the protein prior to administration of a composition described herein or compared to an untreated subject. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months, or more following administration of the composition depending on the dose and route of administration used for treatment. Depending on the outcome of the evaluation, the patient may receive additional treatments.

### Kits

The compositions described herein can be provided in a kit for use in treating vestibular dysfunction. Compositions may include an SLC6A14 promoter described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1), nucleic acid vectors containing such polynucleotides, and nucleic acid vectors containing a polynucleotide described herein operably linked to a transgene encoding a protein of interest (e.g., a protein that can be expressed in VSCs to treat vestibular dysfunction. The nucleic acid vectors may be packaged in an AAV virus capsid (e.g., AAV1, AAV2, AAV2quad(Y-F), AAV3, AAV4, AAV5, AAV6, AAV7, AAV8, AAV9, Anc80, 7m8, PHP.B, PHP.eB, or PHP.S). The kit can further include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Determination of SLC6A14v2 and SLC6A14v3 Promoter Activity in Murine Vestibular Organs In Vivo

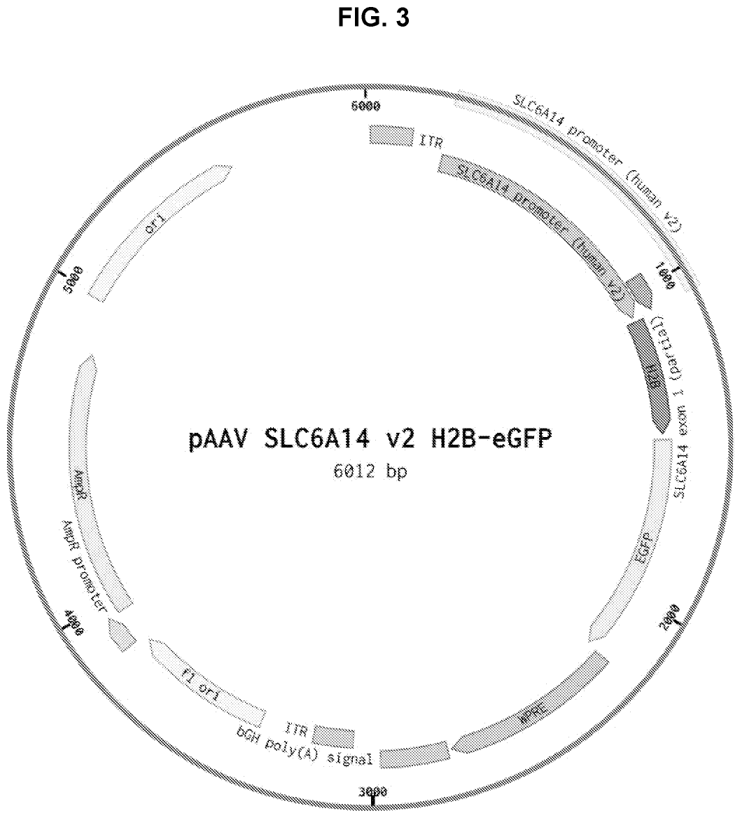

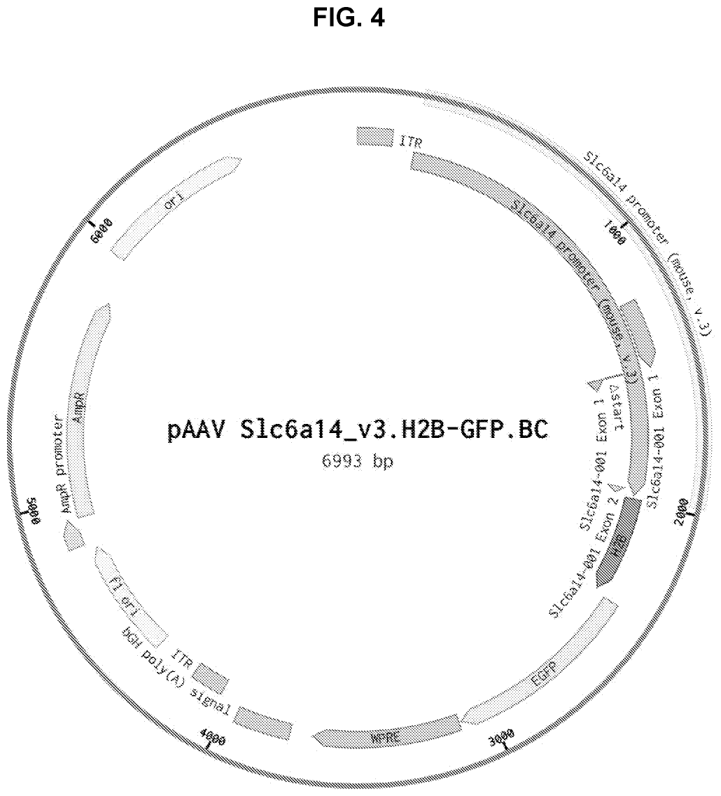

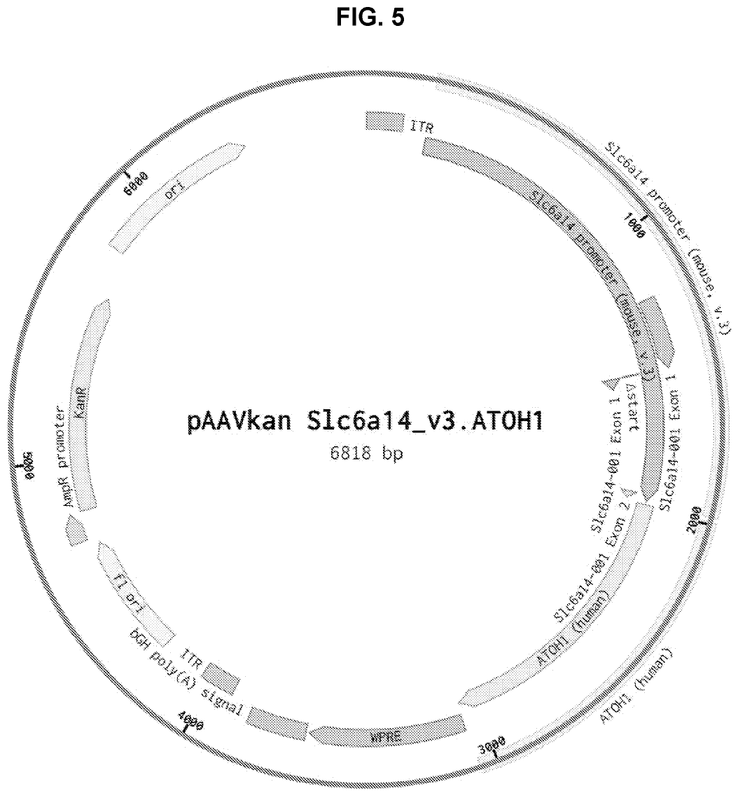

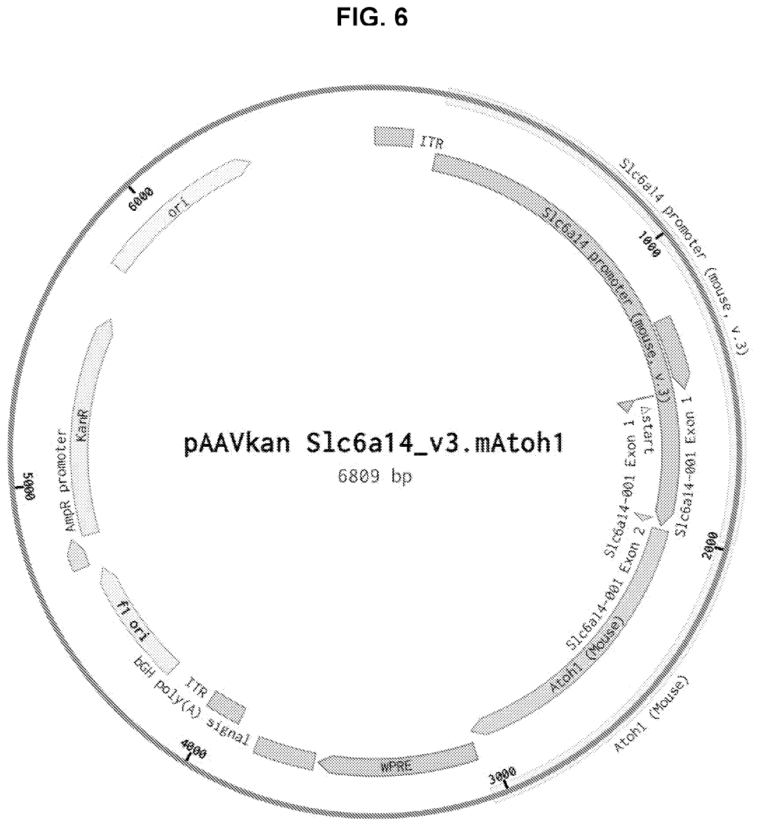

To compare the activity of the SLC6A14v2 and SLC6A14v3 promoters in vivo, a human SLC6A14 promoter (SEQ ID NO: 3) driving nuclear-directed H2B-GFP fusion protein (from plasmid P530; FIG. 3) and a murine SLC6A14 promoter (SEQ ID NO: 1) driving nuclear-directed H2B-GFP fusion protein (from plasmid P919 (SEQ ID NO: 2); FIG. 4) were separately packaged into AAV8 and 1 μL of virus was delivered by injection into the posterior semicircular canal of male eight week-old C57BL/6 mice at a dose of 2.0×10¹⁰ vg/ear (n=6 mice per virus). After two weeks, animals were subsequently euthanized by CO2 and perfused with PBS followed by neutral buffered formalin (NBF). Temporal bones were extracted, utricles were micro-dissected, and fluorescence immunolabeling for the hair cell nuclear marker Pou4f3 (1:200, sc-1980, Santa Cruz Biotechnology, Dallas, Texas, USA) and supporting cell nuclear marker Sox2 (1:200, AF2018, R&D Systems, Inc., Minneapolis, Minnesota, USA) was performed.

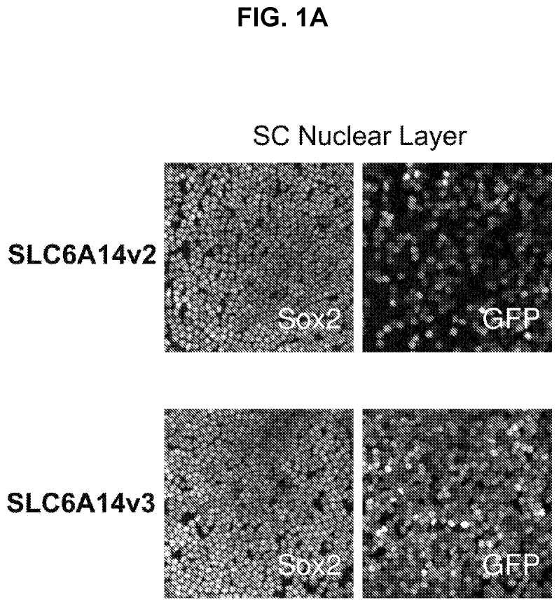

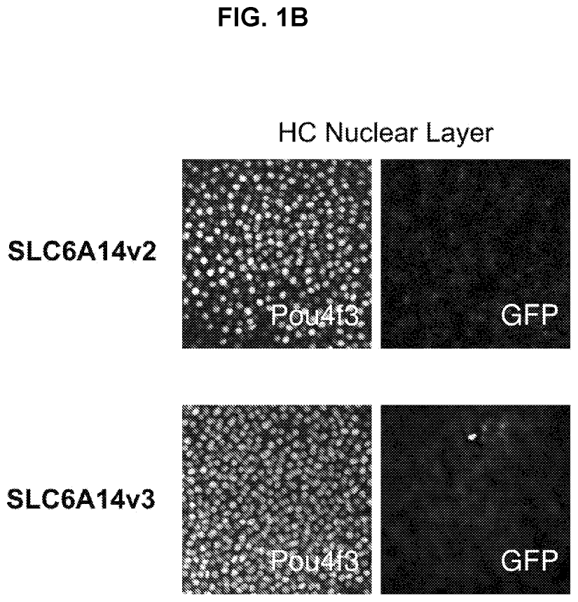

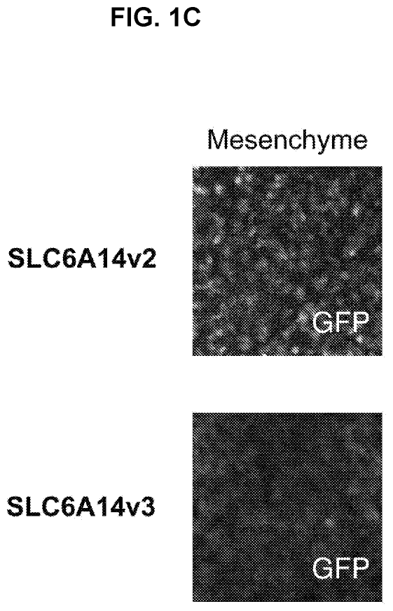

The organs were whole mounted on glass slides and imaged on a Zeiss LSM 800 confocal microscope (FIGS. 1A-1C). For each utricle, a z-stack of confocal images was collected with a 20×/0.8NA objective, which has sufficient field of view to capture the entire utricle. Each z-stack spanned the hair cell nuclear layer, supporting cell nuclear layer, and mesenchymal layer with z-thickness set to the Nyquist criterion. The images in FIGS. 1A-1C show zoomed regions of a single z-plane within the z-stack at the indicated depth. Nuclear GFP expression is shown in the supporting cell nuclear layer within the sensory epithelium (FIG. 1A), the hair cell nuclear layer within the sensory epithelium (FIG. 1B), and in the mesenchymal layer underneath the sensory epithelium (FIG. 1C). Comparable levels of nuclear GFP expression were detected across the supporting cell nuclear layer for both promoters. Nuclear GFP expression was substantially less in the hair cell nuclear layer and mesenchyme; however, more labeling was visible in the mesenchyme with SLC6A14v2 promoter (FIG. 1C, top row) compared to the SLC6A14v3 promoter (FIG. 1C, bottom row).

Quantitative measurements of GFP-expressing nuclei were made using an automated algorithm for 3D counting in Imaris software that determines the number of GFP-positive nuclei within the entire utricle, the intensity of GFP fluorescence, and co-positivity for Pou4f3 and/or Sox2 immunolabeling. Statistical analyses were performed in GraphPad Prism.

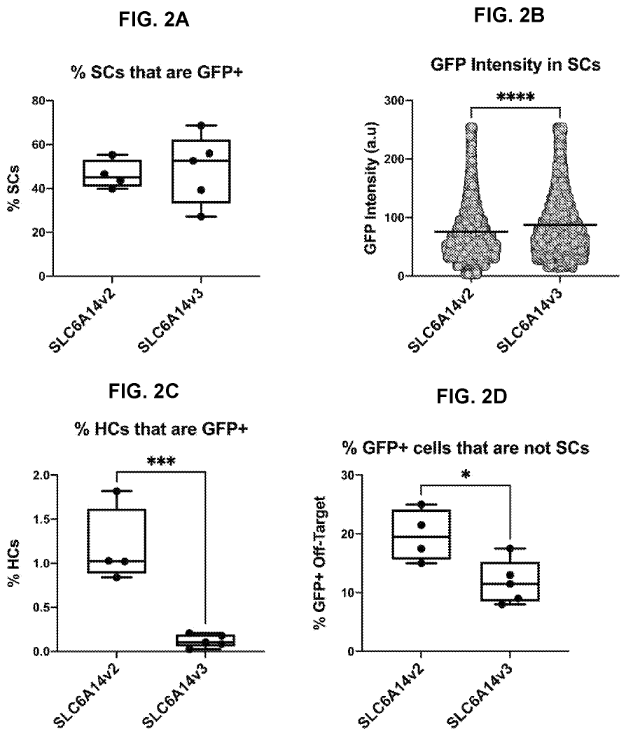

The percentage of supporting cells with detectable levels of nuclear GFP were comparable between the SLC6A14v2 and SLC6A14v3 promoters (FIG. 2A; points on box plots represent individual utricles). However, the average intensity of nuclear GFP in supporting cells with detectable levels above background was significantly greater for the SLC6A14v3 promoter compared to SLC6A14v2 (FIG. 2B; circles on scatter plots represent individual cells across all samples, black lines are the population average; p<0.0001, Student's t-test). In addition, the percentage of hair cell nuclei with detectable levels of GFP above background was significantly higher for the SLC6A14v2 promoter compared to SLC6A14v3 (FIG. 2C; p=0.001, Student's t-test), as was the percentage of all GFP+ nuclei that were not supporting cells as determined by positive Sox2 immunolabeling (FIG. 2D; p=0.022; Student's t-test).

### Example 2. Administration of a Composition Containing a Nucleic Acid Vector Containing an SLC6A14 Promoter to a Subject with Vestibular Dysfunction

According to the methods disclosed herein, a physician of skill in the art can treat a patient, such as a human patient, with vestibular dysfunction so as to improve or restore vestibular function. To this end, a physician of skill in the art can administer to the human patient a composition containing an AAV vector (e.g., AAV1, AAV2, AAV2quad(Y-F), AAV3, AAV4, AAV5, AAV6, AAV7, AAV8, AAV9, Anc80, 7m8, PHP.B, PHP.eB, or PHP.S) containing an SLC6A14 promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1) operably linked to a transgene that encodes a therapeutic protein (e.g., Atonal BHLH Transcription Factor 1 (Atoh1)). In one example, the vector has an AAV8 capsid and contains nucleotides 219-3831 of SEQ ID NO: 10. The composition containing the AAV vector may be administered to the patient, for example, by local administration to the inner ear (e.g., injection into a semicircular canal), to treat vestibular dysfunction.

Following administration of the composition to a patient, a practitioner of skill in the art can monitor the expression of the therapeutic protein encoded by the transgene, and the patient's improvement in response to the therapy, by a variety of methods. For example, a physician can monitor the patient's vestibular function by performing standard tests such as electronystagmography, video nystagmography, VOR tests (e.g., head impulse tests (Halmagyi-Curthoys test, e.g., VHIT), or caloric reflex tests), rotation tests, vestibular evoked myogenic potential, or computerized dynamic posturography. A finding that the patient exhibits improved vestibular function in one or more of the tests following administration of the composition compared to test results obtained prior to administration of the composition indicates that the patient is responding favorably to the treatment. Subsequent doses can be determined and administered as needed.

Exemplary embodiments of the invention are described in the enumerated paragraphs below.

E1. A nucleic acid vector comprising a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1.

E2. The nucleic acid vector of any one of E1, wherein the polynucleotide is operably linked to a transgene.

E3. The nucleic acid vector of E2, wherein the transgene is a heterologous transgene.

E4. The nucleic acid vector of E2 or E3, wherein the transgene encodes a protein, a short hairpin RNA (shRNA), an antisense oligonucleotide (ASO), a nuclease, or a microRNA.

E5. The nucleic acid vector of E4, wherein the polynucleotide is capable of directing vestibular supporting cell (VSC)-specific expression of the protein, shRNA, ASO, nuclease, or microRNA in a mammalian VSC.

E6. The nucleic acid vector of E5, wherein the VSC is a human VSC.

E7. The nucleic acid vector of any one of E4-E6, wherein the protein is Spalt Like Transcription Factor 2 (Sall2), Calmodulin Binding Transcription Activator 1 (Camta1), Hes Related Family BHLH Transcription Factor With YRPW Motif 2 (Hey2), Gata Binding Protein 2 (Gata2), Hes Related Family BHLH Transcription Factor With YRPW Motif 1 (Hey1), Ceramide Synthase 2 (Lass2), SRY-Box 10 (Sox10), GATA Binding Protein 3 (Gata3), Cut Like Homeobox 1 (Cux1), Nuclear Receptor Subfamily 2 Group F Member (Nr2f1), Hes Related Family BHLH Transcription Factor (Hes1), RAR Related Orphan Receptor B (Rorb), Jun Proto-Oncogene AP-1 Transcription Factor Subunit (Jun), Zinc Finger Protein 667 (Zfp667), LIM Homeobox 3 (Lhx3), Nescient Helix-Loop-Helix 1 (Nhlh1), MAX Dimerization Protein 4 (Mxd4), Zinc Finger MIZ-Type Containing 1 (Zmiz1), Myelin Transcription Factor 1 (Myt1), Signal Transducer And Activator Of Transcription 3 (Stat3), BarH Like Homeobox 1 (Barhl1), Thymocyte Selection Associated High Mobility Group Box (Tox), Prospero Homeobox 1 (Prox1), Nuclear Factor I A (Nfia), Thyroid Hormone Receptor Beta (Thrb), MYCL Proto-Oncogene BHLH Transcription Factor (Mycl1), Lysine Demethylase 5A (Kdm5a), CAMP Responsive Element Binding Protein 3 Like 4 (Creb314), ETS Variant 1 (Etv1), Paternally Expressed 3 (Peg3), BTB Domain And CNC Homolog 2 (Bach2), ISL LIM Homeobox 1 (Isl1), Zinc Finger And BTB Domain Containing 38 (Zbtb38), Limb Bud And Heart Development (Lbh), Tubby Bipartite Transcription Factor (Tub), Ubiquitin C (Hmg20), RE1 Silencing Transcription Factor (Rest), Zinc Finger Protein 827 (Zfp827), AF4/FMR2 Family Member 3 (Aff3), PBX/Knotted 1 Homeobox 2 (Pknox2), AT-Rich Interaction Domain 3B (Arid3b), MLX Interacting Protein (Mixip), Zinc Finger Protein (Zfp532), IKAROS Family Zinc Finger 2 (Ikzf2), Spalt Like Transcription Factor 1 (Sall1), SIX Homeobox 2 (Six2), Spalt Like Transcription Factor 3 (Sall3), Lin-28 Homolog B (Lin28b), Regulatory Factor X7 (Rfx7), Brain Derived Neurotrophic Factor (Bdnf), Growth Factor Independent 1 Transcriptional Repressor (Gfi1), POU Class 4 Homeobox 3 (Pou4f3), MYC Proto-Oncogene BHLH Transcription Factor (Myc), β-catenin (Ctnnb1), SRY-Box 2 (Sox2), SRY-Box 4 (Sox4), SRY-Box 11 (Sox11), TEA Domain Transcription Factor 2 (Tead2), Atonal BHLH Transcription Factor 1 (Atoh1), or an Atoh1 variant.

E8. The nucleic acid vector of E7, wherein the protein is Atoh1.

E9. The nucleic acid vector of E7, wherein the Atoh1 variant has one or more amino acid substitutions selected from the group consisting of S328A, S331A, S334A, S328A/S331A, S328A/S334A, S331A/S334A, and S328A/S331A/S334.

E10. The nucleic acid vector of any one of E2-E9, wherein the nucleic acid vector additionally comprises a first inverted terminal repeat 5′ of the polynucleotide; and, 3′ of the transgene and in 5′ to 3′ order, an optional posttranscriptional regulatory element, a polyadenylation signal, and a second inverted terminal repeat.

E11. The nucleic acid vector of E10, comprising nucleotides 219-3831 of SEQ ID NO: 10, a first inverted terminal repeat 5′ of nucleotides 219-3831 of SEQ ID NO: 10, wherein the 5′ inverted terminal repeat has at least 80% sequence identity to nucleotides 1-130 of SEQ ID NO: 10; and a second inverted terminal repeat 3′ of nucleotides 219-3831 of SEQ ID NO: 10, wherein the 3′ inverted terminal repeat has at least 80% sequence identity to nucleotides 3919-4048 of SEQ ID NO: 10.

E12. The nucleic acid vector of E10, comprising nucleotides 219-3822 of SEQ ID NO: 11, a first inverted terminal repeat 5′ of nucleotides 219-3822 of SEQ ID NO: 11, wherein the 5′ inverted terminal repeat has at least 80% sequence identity to nucleotides 1-130 of SEQ ID NO: 11; and a second inverted terminal repeat 3′ of nucleotides 219-3822 of SEQ ID NO: 11, wherein the 3′ inverted terminal repeat has at least 80% sequence identity to nucleotides 3910-4039 of SEQ ID NO: 11.

E13. The nucleic acid vector of any one of E1-E12, wherein the nucleic acid vector is a viral vector, plasmid, cosmid, or artificial chromosome.

E14. The nucleic acid vector of E13, wherein the nucleic acid vector is a viral vector selected from the group consisting of an adeno-associated virus (AAV), an adenovirus, and a lentivirus.

E15. The nucleic acid vector of E14, wherein the viral vector is an AAV vector.

E16. The nucleic acid vector of E15, wherein the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S capsid.

E17. A composition comprising the nucleic acid vector of any one of E1-E16.

E18. The composition of E17, further comprising a pharmaceutically acceptable carrier, diluent, or excipient.

E19. A polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1 operably linked to a transgene.

E20. The polynucleotide of E19, wherein the transgene is a heterologous transgene.

E21. The polynucleotide of E20, wherein the transgene encodes a protein, an shRNA, an ASO, a nuclease, or a microRNA.

E22. The polynucleotide of E21, wherein the protein is Sox9, Sall2, Camta1, Hey2, Gata2, Hey1, Lass2, Sox10, Gata3, Cux1, Nr2f1, Hes1, Rorb, Jun, Zfp667, Lhx3, Nhlh1, Mxd4, Zmiz1, Myt1, Stat3, Barhl1, Tox, Prox1, Nfia, Thrb, Mycl1, Kdm5a, Creb314, Etv1, Peg3, Bach2, Isl1, Zbtb38, Lbh, Tub, Hmg20, Rest, Zfp827, Aff3, Pknox2, Arid3b, Mixip, Zfp532, Ikzf2, Sall1, Six2, Sall3, Lin28b, Rfx7, Bdnf, Gfi1, Pou4f3, Myc, Ctnnb1, Sox2, Sox4, Sox11, Tead2, Atoh1, or an Atoh1 variant.

E23. The polynucleotide of E22, wherein the protein is Atoh1.

E24. A cell comprising the polynucleotide of any one of E19-E23 or the nucleic acid vector of any one of E1-E16.

E25. The cell of E24, wherein the cell is a mammalian VSC.

E26. The cell of E25, wherein the mammalian VSC is a human VSC.

E27. A method of expressing a transgene in a mammalian VSC, comprising contacting the mammalian VSC with the nucleic acid vector of any one of E1-E16 or the composition of E17 or E18.

E28. The method of E27, wherein the transgene is specifically expressed in VSCs.

E29. The method of E27 or E28, wherein the mammalian VSC is a human VSC.

E30. A method of treating a subject having or at risk of developing vestibular dysfunction, comprising administering to the subject an effective amount of the nucleic acid vector of any one of E1-E16 or the composition of E17 or E18.

E31. The method of E30, wherein the vestibular dysfunction comprises vertigo, dizziness, imbalance, bilateral vestibulopathy (also known as bilateral vestibular hypofunction), oscillopsia, or a balance disorder.

E32. The method of E30 or E31, wherein the vestibular dysfunction is age-related vestibular dysfunction, head trauma-related vestibular dysfunction, disease or infection-related vestibular dysfunction, or ototoxic drug-induced vestibular dysfunction.

E33. The method of any one of E30-E32, wherein the vestibular dysfunction is associated with a genetic mutation.

E34. The method of E30 or E31, wherein the vestibular dysfunction is idiopathic vestibular dysfunction.

E35. A method of inducing or increasing vestibular hair cell regeneration in a subject in need thereof, comprising administering to the subject an effective amount of the nucleic acid vector of any one of E1-E16 or the composition of E17 or E18.

E36. A method of inducing or increasing VSC proliferation in a subject in need thereof, comprising administering to the subject an effective amount of the nucleic acid vector of any one of E1-E16 or the composition of E17 or E18.

E37. A method of inducing or increasing vestibular hair cell proliferation in a subject in need thereof, comprising administering to the subject an effective amount of the nucleic acid vector of any one of E1-E16 or the composition of E17 or E18.

E38. A method of inducing or increasing vestibular hair cell maturation (e.g., the maturation of regenerated hair cells) in a subject in need thereof, the method comprising administering to the subject an effective amount of the nucleic acid vector of any one of E1-E16 or the composition of E17 or E18.

E39. A method of inducing or increasing vestibular hair cell innervation in a subject in need thereof, the method comprising administering to the subject an effective amount of the nucleic acid vector of any one of E1-E16 or the composition of E17 or E18.

E40. A method of increasing VSC and/or vestibular hair cell survival in a subject in need thereof, the method comprising administering to the subject an effective amount of the nucleic acid vector of any one of E1-E16 or the composition of E17 or E18.

E41. The method of any one of E35-E40, wherein the subject has or is at risk of developing vestibular dysfunction (e.g., vertigo, dizziness, imbalance, bilateral vestibulopathy (bilateral vestibular hypofunction), oscillopsia, or a balance disorder).

E42. A method of treating a subject having or at risk of developing bilateral vestibulopathy (also known as bilateral vestibular hypofunction), the method comprising administering to the subject an effective amount of the nucleic acid vector of any one of E1-E16 or the composition of E17 or E18.

E43. A method of treating a subject having or at risk of developing oscillopsia, the method comprising administering to the subject an effective amount of the nucleic acid vector of any one of E1-E16 or the composition of E17 or E18.

E44. The method of E42 or E43, wherein the bilateral vestibulopathy or the oscillopsia is ototoxic drug-induced bilateral vestibulopathy or ototoxic drug-induced oscillopsia.

E45. The method of E32 or E44, wherein the ototoxic drug is selected from the group consisting of aminoglycosides, antineoplastic drugs, ethacrynic acid, furosemide, salicylates, and quinine.

E46. A method of treating a subject having or at risk of developing a balance disorder, the method comprising administering to the subject an effective amount of the nucleic acid vector of any one of E1-E16 or the composition of E17 or E18.

E47. The method of any one of E30-E46, wherein the method further comprises evaluating the vestibular function of the subject prior to administering the nucleic acid vector or composition.

E48. The method of any one of E30-E47, wherein the method further comprises evaluating the vestibular function of the subject after administering the nucleic acid vector or composition.

E49. The method of any one of E30-E48, wherein the nucleic acid vector or composition is locally administered.

E50. The method of E49, wherein the nucleic acid vector or composition is administered to a semicircular canal.

E51. The method of E49, wherein the nucleic acid vector or composition is administered transtympanically or intratympanically.

E52. The method of E49, wherein the nucleic acid vector or composition is administered into the perilymph.

E53. The method of E49, wherein the nucleic acid vector or composition is administered into the endolymph.

E54. The method of E49, wherein the nucleic acid vector or composition is administered to or through the oval window.

E55. The method of E49, wherein the nucleic acid vector or composition is administered to or through the round window.

E56. The method of any one of E30-E55, wherein the nucleic acid vector or composition is administered in an amount sufficient to prevent or reduce vestibular dysfunction, delay the development of vestibular dysfunction, slow the progression of vestibular dysfunction, improve vestibular function, increase vestibular hair cell numbers, increase vestibular hair cell maturation (e.g., the maturation of regenerated hair cells), increase vestibular hair cell proliferation, increase vestibular hair cell regeneration, increase vestibular hair cell innervation, increase VSC proliferation, increase VSC numbers, increase VSC survival, increase vestibular hair cell survival, or improve VSC function.

E57. The method of any one of E30-E56, wherein the subject is a human.

E58. A kit comprising the nucleic acid vector of any one of E1-E16 or the composition of E17 or E18.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

1. A nucleic acid vector comprising a Solute Carrier Family 6 Member 14 (SLC6A14) promoter comprising a polynucleotide sequence having at least 85% sequence identity to SEQ ID NO: 1.

2. The nucleic acid vector of claim 1, wherein the SLC6A14 promoter has the sequence of SEQ ID NO: 1.

3. The nucleic acid vector of claim 1 or 2, wherein the SLC6A14 promoter is operably linked to a transgene.

4. The nucleic acid vector of claim 3, wherein the transgene is a heterologous transgene.

5. The nucleic acid vector of claim 3 or 4, wherein the transgene encodes a protein, a short hairpin RNA (shRNA), an antisense oligonucleotide (ASO), a nuclease, or a microRNA.

6. The nucleic acid vector of claim 5, wherein the transgene encodes a protein.

7. The nucleic acid vector of claim 6, wherein the protein is Atonal BHLH Transcription Factor 1 (Atoh1), Spalt Like Transcription Factor 2 (Sall2), Calmodulin Binding Transcription Activator 1 (Camta1), Hes Related Family BHLH Transcription Factor With YRPW Motif 2 (Hey2), Gata Binding Protein 2 (Gata2), Hes Related Family BHLH Transcription Factor With YRPW Motif 1 (Hey1), Ceramide Synthase 2 (Lass2), SRY-Box 10 (Sox10), GATA Binding Protein 3 (Gata3), Cut Like Homeobox 1 (Cux1), Nuclear Receptor Subfamily 2 Group F Member (Nr2f1), Hes Related Family BHLH Transcription Factor (Hes1), RAR Related Orphan Receptor B (Rorb), Jun Proto-Oncogene AP-1 Transcription Factor Subunit (Jun), Zinc Finger Protein 667 (Zfp667), LIM Homeobox 3 (Lhx3), Nescient Helix-Loop-Helix 1 (Nhlh1), MAX Dimerization Protein 4 (Mxd4), Zinc Finger MIZ-Type Containing 1 (Zmiz1), Myelin Transcription Factor 1 (Myt1), Signal Transducer And Activator Of Transcription 3 (Stat3), BarH Like Homeobox 1 (Barhl1), Thymocyte Selection Associated High Mobility Group Box (Tox), Prospero Homeobox 1 (Prox1), Nuclear Factor I A (Nfia), Thyroid Hormone Receptor Beta (Thrb), MYCL Proto-Oncogene BHLH Transcription Factor (Mycl1), Lysine Demethylase 5A (Kdm5a), CAMP Responsive Element Binding Protein 3 Like 4 (Creb314), ETS Variant 1 (Etv1), Paternally Expressed 3 (Peg3), BTB Domain And CNC Homolog 2 (Bach2), ISL LIM Homeobox 1 (Isl1), Zinc Finger And BTB Domain Containing 38 (Zbtb38), Limb Bud And Heart Development (Lbh), Tubby Bipartite Transcription Factor (Tub), Ubiquitin C (Hmg20), RE1 Silencing Transcription Factor (Rest), Zinc Finger Protein 827 (Zfp827), AF4/FMR2 Family Member 3 (Aff3), PBX/Knotted 1 Homeobox 2 (Pknox2), AT-Rich Interaction Domain 3B (Arid3b), MLX Interacting Protein (Mixip), Zinc Finger Protein (Zfp532), IKAROS Family Zinc Finger 2 (Ikzf2), Spalt Like Transcription Factor 1 (Sall1), SIX Homeobox 2 (Six2), Spalt Like Transcription Factor 3 (Sall3), Lin-28 Homolog B (Lin28b), Regulatory Factor X7 (Rfx7), Brain Derived Neurotrophic Factor (Bdnf), Growth Factor Independent 1 Transcriptional Repressor (Gfi1), POU Class 4 Homeobox 3 (Pou4f3), MYC Proto-Oncogene BHLH Transcription Factor (Myc), β-catenin (Ctnnb1), SRY-Box 2 (Sox2), SRY-Box 4 (Sox4), SRY-Box 11 (Sox11), TEA Domain Transcription Factor 2 (Tead2), or an Atoh1 variant.

8. The nucleic acid vector of claim 7, wherein the protein is Atoh1.

9. The nucleic acid vector of any one of claims 3-8, wherein the nucleic acid vector additionally comprises a first inverted terminal repeat 5′ of the SLC6A14 promoter; and, 3′ of the transgene and in 5′ to 3′ order, an optional posttranscriptional regulatory element, a polyadenylation signal, and a second inverted terminal repeat.

10. The nucleic acid vector of claim 9, comprising nucleotides 219-3831 of SEQ ID NO: 10, a first inverted terminal repeat 5′ of nucleotides 219-3831 of SEQ ID NO: 10, wherein the 5′ inverted terminal repeat has at least 80% sequence identity to nucleotides 1-130 of SEQ ID NO: 10; and a second inverted terminal repeat 3′ of nucleotides 219-3831 of SEQ ID NO: 10, wherein the 3′ inverted terminal repeat has at least 80% sequence identity to nucleotides 3919-4048 of SEQ ID NO: 10.

11. The nucleic acid vector of claim 9, comprising nucleotides 219-3822 of SEQ ID NO: 11, a first inverted terminal repeat 5′ of nucleotides 219-3822 of SEQ ID NO: 11, wherein the 5′ inverted terminal repeat has at least 80% sequence identity to nucleotides 1-130 of SEQ ID NO: 11; and a second inverted terminal repeat 3′ of nucleotides 219-3822 of SEQ ID NO: 11, wherein the 3′ inverted terminal repeat has at least 80% sequence identity to nucleotides 3910-4039 of SEQ ID NO: 11.

12. The nucleic acid vector of any one of claims 1-11, wherein the nucleic acid vector is a plasmid.

13. The nucleic acid vector of any one of claims 1-11, wherein the nucleic acid vector is an adeno-associated virus (AAV) vector.

14. The nucleic acid vector of claim 13, wherein the AAV vector has an AAV8 capsid.

15. A pharmaceutical composition comprising the nucleic acid vector of any one of claims 1-14 and a pharmaceutically acceptable carrier, diluent, or excipient.

16. A method of expressing a transgene in a mammalian vestibular supporting cell (VSC), comprising contacting the mammalian VSC with the nucleic acid vector of any one of claims 1-14 or the composition of claim 15.

17. The method of claim 16, wherein the mammalian VSC is a human VSC.

18. A method of treating a subject having or at risk of developing vestibular dysfunction, comprising administering to an inner ear of the subject an effective amount of the nucleic acid vector of any one of claims 1-14 or the composition of claim 15.

19. A method of inducing or increasing vestibular hair cell regeneration in a subject in need thereof, comprising administering to an inner ear of the subject an effective amount of the nucleic acid vector of any one of claims 1-14 or the composition of claim 15.

20. A method of treating a subject having or at risk of developing bilateral vestibulopathy, the method comprising administering to an inner ear of the subject an effective amount of the nucleic acid vector of any one of claims 1-14 or the composition of claim 15.


SEQUENCE LISTING (11 sequences)

SEQ ID NO: 1 - DNA, 1759 bp, Mus musculus
atacacttatgtatatgtgcgatgtcagtgtgtgtgcatataaagtcccaaacaagcctgtatgatattgaccaacaaggtcaaggcaaagttttgatactttcaggtcacaacctctccgcatccctctctactttgctctatctgcctgaactcctgaggacatgtttctactgcaaatggaaaatccttgtcagccagtgaggaacaaagggactatacatagatgaaaacttggctctctgctggttcctttgtttgtatgaatttatacaatttggtaaaactgccaccatgtcttacatggacagattgagtgtagattctttgaatttttgatgaagaggcgctgcactggtgatcggaattgcagtctttcctctgtaggtaacctggcttgtttccttacagtttactttctaggcctcgcctttctcacagagtgaagtcctttgttaaggttcgaatttcccataaacctgctcaataatttgtttgtgtttggcttctttgaaatactacacaaagcaatccttgtaaaaggcaaaactattccgaaggctgagaaaggagctccaggacatagattcaaagtcgctcttttcaggtagagacagctgggtaatcttatcttaactggctacatttcaaggttcccaattcaggggctttcccctctgggagcagcattctctccgggtgatgaagagctttctagtgaggagcaaaactttcagaaaaccggagggcccagagcagtctggtctgttcacaaaaattatagcaaacaaaataagcccggcggattgggtctctcctacctccagcaccaggggagatcagcacttggccccaggacagagacctgagaagtgaggtttggaagaagccaggaatccaggaaaggaggcaagattgctaaggcaccggcacagctctgagtcaaaagttgtcagtcttctttggctctggctgcggagctcaattgctcacagccctgccctttcctagggctggggcaaggaattgctacattcaggattacctgggggaaaaaccagaggcttgctttggtcccttccggtaattgaaaggactggccgtcagcgagggggaggagagagcttccctccataaatggtcccacccctgggcaaggtggctcactttggcaggtagcaaccggggagtgtgcacctgccaccagtcaagctcagccagactgtgagaagaggagaggcgaggcacaccaagggatccagtgaaccaacgacagattgaagtgcccgaacttcttcaagtgcagacagaaggaggtagggttctggaagtttctggtggtgtaggggagtcaggaagggaaaacaaggagggagagtgagtcttagttttttgctttctgtagctgttccttattttgcatatttctttctcttcaactcttttcaagtatgcctgatacgttgttctcacgaagttgacgtgaaaaacaactttcctgctggtagttaggaaacttaggagcacctcaacctgtaccttgagaacacccagagaatgctgctctttgtcgttctctataccgtgttcatatgctgcagggaaatgcaaagaatgtactgtccttatctgaccctgggagcattccatagtcaagcagcagctatcaggttgggaaagagctcctctccaaggtgtaacagaaaaggaaaatgttgatatttttcttgtttagaaagtgacagcttcatccgagaac

SEQ ID NO: 2 - DNA, 6993 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccatacacttatgtatatgtgcgatgtcagtgtgtgtgcatataaagtcccaaacaagcctgtatgatattgaccaacaaggtcaaggcaaagttttgatactttcaggtcacaacctctccgcatccctctctactttgctctatctgcctgaactcctgaggacatgtttctactgcaaatggaaaatccttgtcagccagtgaggaacaaagggactatacatagatgaaaacttggctctctgctggttcctttgtttgtatgaatttatacaatttggtaaaactgccaccatgtcttacatggacagattgagtgtagattctttgaatttttgatgaagaggcgctgcactggtgatcggaattgcagtctttcctctgtaggtaacctggcttgtttccttacagtttactttctaggcctcgcctttctcacagagtgaagtcctttgttaaggttcgaatttcccataaacctgctcaataatttgtttgtgtttggcttctttgaaatactacacaaagcaatccttgtaaaaggcaaaactattccgaaggctgagaaaggagctccaggacatagattcaaagtcgctcttttcaggtagagacagctgggtaatcttatcttaactggctacatttcaaggttcccaattcaggggctttcccctctgggagcagcattctctccgggtgatgaagagctttctagtgaggagcaaaactttcagaaaaccggagggcccagagcagtctggtctgttcacaaaaattatagcaaacaaaataagcccggcggattgggtctctcctacctccagcaccaggggagatcagcacttggccccaggacagagacctgagaagtgaggtttggaagaagccaggaatccaggaaaggaggcaagattgctaaggcaccggcacagctctgagtcaaaagttgtcagtcttctttggctctggctgcggagctcaattgctcacagccctgccctttcctagggctggggcaaggaattgctacattcaggattacctgggggaaaaaccagaggcttgctttggtcccttccggtaattgaaaggactggccgtcagcgagggggaggagagagcttccctccataaatggtcccacccctgggcaaggtggctcactttggcaggtagcaaccggggagtgtgcacctgccaccagtcaagctcagccagactgtgagaagaggagaggcgaggcacaccaagggatccagtgaaccaacgacagattgaagtgcccgaacttcttcaagtgcagacagaaggaggtagggttctggaagtttctggtggtgtaggggagtcaggaagggaaaacaaggagggagagtgagtcttagttttttgctttctgtagctgttccttattttgcatatttctttctcttcaactcttttcaagtatgcctgatacgttgttctcacgaagttgacgtgaaaaacaactttcctgctggtagttaggaaacttaggagcacctcaacctgtaccttgagaacacccagagaatgctgctctttgtcgttctctataccgtgttcatatgctgcagggaaatgcaaagaatgtactgtccttatctgaccctgggagcattccatagtcaagcagcagctatcaggttgggaaagagctcctctccaaggtgtaacagaaaaggaaaatgttgatatttttcttgtttagaaagtgacagcttcatccgagaacgcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgaacaattgcatcggacacatcttggcgttttacaacgtcgtgactgggaaaaccctggcgttacccaacttaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 3 - DNA, 834 bp, Homo sapiens
aagctgggatgtttcctcatagtttactttctaggcctcatctttcttacagagtgtgctcctttgttaaggttagaatttcccataaacctgctcaataatttgtttgtgtttggcttctttgaaatactacacaaagcaatccctgtaaaaggcaaagctgtcctgaaggctgagaaaggagcctgagacataggctccaagttgctcttttcaggcagagccagctgggtaatcttatctcagatggctgcttttcaaggtgcccaattcaggggcttttcctctgggagcagcatttgccccagggaatcaagtgctttctagtcaggggcaaaactttgggaaatctgaggacccagggtggtatggtctgttcaggagaattttggggaacagaatggcccccttctccctccagcacttgtacagatcagcacttggccccagaacagagaccagactgagaggcgaggttaggaggaaacaggggacccaggaaaggcggctagattgcaaacgtacctacacagctctgagtcaaaggctgtcagtcatctcggctcagactgctctgctctccagcagcccagccctttcccagggctggggcaggagattgctacatgtaggcttatctggggaaaaaccagagcctcactttagtcccttccggtaattgacactactggacacccaggagggggaggagagagcttctcttcataaatgttcccacccctgggcaaggtggctcactctggcaggtaggaacaggggagagtgcacctgctaccagtcaagctcagccagactgcaagaggaggcgaggcg

SEQ ID NO: 4 - protein, 354 aa, Homo sapiens
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENSKTSPRSHRSDGEFSPHSHYSDSDEAS

SEQ ID NO: 5 - DNA, 1062 bp, Homo sapiens
atgtcccgcctgctgcatgcagaagagtgggctgaagtgaaggagttgggagaccaccatcgccagccccagccgcatcatctcccgcaaccgccgccgccgccgcagccacctgcaactttgcaggcgagagagcatcccgtctacccgcctgagctgtccctcctggacagcaccgacccacgcgcctggctggctcccactttgcagggcatctgcacggcacgcgccgcccagtatttgctacattccccggagctgggtgcctcagaggccgctgcgccccgggacgaggtggacggccggggggagctggtaaggaggagcagcggcggtgccagcagcagcaagagccccgggccggtgaaagtgcgggaacagctgtgcaagctgaaaggcggggtggtggtagacgagctgggctgcagccgccaacgggccccttccagcaaacaggtgaatggggtgcagaagcagagacggctagcagccaacgccagggagcggcgcaggatgcatgggctgaaccacgccttcgaccagctgcgcaatgttatcccgtcgttcaacaacgacaagaagctgtccaaatatgagaccctgcagatggcccaaatctacatcaacgccttgtccgagctgctacaaacgcccagcggaggggaacagccaccgccgcctccagcctcctgcaaaagcgaccaccaccaccttcgcaccgcggcctcctatgaagggggcgcgggcaacgcgaccgcagctggggctcagcaggcttccggagggagccagcggccgaccccgcccgggagttgccggactcgcttctcagccccagcttctgcgggagggtactcggtgcagctggacgctctgcacttctcgactttcgaggacagcgccctgacagcgatgatggcgcaaaagaatttgtctccttctctccccgggagcatcttgcagccagtgcaggaggaaaacagcaaaacttcgcctcggtcccacagaagcgacggggaattttccccccattcccattacagtgactcggatgaggcaagt

SEQ ID NO: 6 - protein, 351 aa, Mus musculus
MSRLLHAEEWAEVKELGDHHRHPQPHHVPPLTPQPPATLQARDLPVYPAELSLLDSTDPRAWLTPTLQGLCTARAAQYLLHSPELGASEAAAPRDEADSQGELVRRSGCGGLSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPNVGEQPPPPTASCKNDHHHLRTASSYEGGAGASAVAGAQPAPGGGPRPTPPGPCRTRFSGPASSGGYSVQLDALHFPAFEDRALTAMMAQKDLSPSLPGGILQPVQEDNSKTSPRSHRSDGEFSPHSHYSDSDEAS

SEQ ID NO: 7 - DNA, 1053 bp, Mus musculus
atgtcccgcctgctgcatgcagaagagtgggctgaggtaaaagagttgggggaccaccatcgccatccccagccgcaccacgtcccgccgctgacgccacagccacctgctaccctgcaggcgagagaccttcccgtctacccggcagaactgtccctcctggatagcaccgacccacgcgcctggctgactcccactttgcagggcctctgcacggcacgcgccgcccagtatctgctgcattctcccgagctgggtgcctccgaggccgcggcgccccgggacgaggctgacagccagggtgagctggtaaggagaagcggctgtggcggcctcagcaagagccccgggcccgtcaaagtacgggaacagctgtgcaagctgaagggtggggttgtagtggacgagcttggctgcagccgccagcgagccccttccagcaaacaggtgaatggggtacagaagcaaaggaggctggcagcaaacgcaagggaacggcgcaggatgcacgggctgaaccacgccttcgaccagctgcgcaacgttatcccgtccttcaacaacgacaagaagctgtccaaatatgagaccctacagatggcccagatctacatcaacgctctgtcggagttgctgcagactcccaatgtcggagagcaaccgccgccgcccacagcttcctgcaaaaatgaccaccatcaccttcgcaccgcctcctcctatgaaggaggtgcgggcgcctctgcggtagctggggctcagccagccccgggagggggcccgagacctaccccgcccgggccttgccggactcgcttctcaggcccagcttcctctgggggttactcggtgcagctggacgctttgcacttcccagccttcgaggacagggccctaacagcgatgatggcacagaaggacctgtcgccttcgctgcccgggggcatcctgcagcctgtacaggaggacaacagcaaaacatctcccagatcccacagaagtgacggagagttttccccccactctcattacagtgactctgatgaggccagt

SEQ ID NO: 8 - DNA, 548 bp, Artificial Sequence
  Synthetic Construct
gatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcga

SEQ ID NO: 9 - DNA, 425 bp, Artificial Sequence
  Synthetic Construct
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaa

SEQ ID NO: 10 - DNA, 6818 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccatacacttatgtatatgtgcgatgtcagtgtgtgtgcatataaagtcccaaacaagcctgtatgatattgaccaacaaggtcaaggcaaagttttgatactttcaggtcacaacctctccgcatccctctctactttgctctatctgcctgaactcctgaggacatgtttctactgcaaatggaaaatccttgtcagccagtgaggaacaaagggactatacatagatgaaaacttggctctctgctggttcctttgtttgtatgaatttatacaatttggtaaaactgccaccatgtcttacatggacagattgagtgtagattctttgaatttttgatgaagaggcgctgcactggtgatcggaattgcagtctttcctctgtaggtaacctggcttgtttccttacagtttactttctaggcctcgcctttctcacagagtgaagtcctttgttaaggttcgaatttcccataaacctgctcaataatttgtttgtgtttggcttctttgaaatactacacaaagcaatccttgtaaaaggcaaaactattccgaaggctgagaaaggagctccaggacatagattcaaagtcgctcttttcaggtagagacagctgggtaatcttatcttaactggctacatttcaaggttcccaattcaggggctttcccctctgggagcagcattctctccgggtgatgaagagctttctagtgaggagcaaaactttcagaaaaccggagggcccagagcagtctggtctgttcacaaaaattatagcaaacaaaataagcccggcggattgggtctctcctacctccagcaccaggggagatcagcacttggccccaggacagagacctgagaagtgaggtttggaagaagccaggaatccaggaaaggaggcaagattgctaaggcaccggcacagctctgagtcaaaagttgtcagtcttctttggctctggctgcggagctcaattgctcacagccctgccctttcctagggctggggcaaggaattgctacattcaggattacctgggggaaaaaccagaggcttgctttggtcccttccggtaattgaaaggactggccgtcagcgagggggaggagagagcttccctccataaatggtcccacccctgggcaaggtggctcactttggcaggtagcaaccggggagtgtgcacctgccaccagtcaagctcagccagactgtgagaagaggagaggcgaggcacaccaagggatccagtgaaccaacgacagattgaagtgcccgaacttcttcaagtgcagacagaaggaggtagggttctggaagtttctggtggtgtaggggagtcaggaagggaaaacaaggagggagagtgagtcttagttttttgctttctgtagctgttccttattttgcatatttctttctcttcaactcttttcaagtatgcctgatacgttgttctcacgaagttgacgtgaaaaacaactttcctgctggtagttaggaaacttaggagcacctcaacctgtaccttgagaacacccagagaatgctgctctttgtcgttctctataccgtgttcatatgctgcagggaaatgcaaagaatgtactgtccttatctgaccctgggagcattccatagtcaagcagcagctatcaggttgggaaagagctcctctccaaggtgtaacagaaaaggaaaatgttgatatttttcttgtttagaaagtgacagcttcatccgagaacgcggccgcgccaccatgtcccgcctgctgcatgcagaagagtgggctgaagtgaaggagttgggagaccaccatcgccagccccagccgcatcatctcccgcaaccgccgccgccgccgcagccacctgcaactttgcaggcgagagagcatcccgtctacccgcctgagctgtccctcctggacagcaccgacccacgcgcctggctggctcccactttgcagggcatctgcacggcacgcgccgcccagtatttgctacattccccggagctgggtgcctcagaggccgctgcgccccgggacgaggtggacggccggggggagctggtaaggaggagcagcggcggtgccagcagcagcaagagccccgggccggtgaaagtgcgggaacagctgtgcaagctgaaaggcggggtggtggtagacgagctgggctgcagccgccaacgggccccttccagcaaacaggtgaatggggtgcagaagcagagacggctagcagccaacgccagggagcggcgcaggatgcatgggctgaaccacgccttcgaccagctgcgcaatgttatcccgtcgttcaacaacgacaagaagctgtccaaatatgagaccctgcagatggcccaaatctacatcaacgccttgtccgagctgctacaaacgcccagcggaggggaacagccaccgccgcctccagcctcctgcaaaagcgaccaccaccaccttcgcaccgcggcctcctatgaagggggcgcgggcaacgcgaccgcagctggggctcagcaggcttccggagggagccagcggccgaccccgcccgggagttgccggactcgcttctcagccccagcttctgcgggagggtactcggtgcagctggacgctctgcacttctcgactttcgaggacagcgccctgacagcgatgatggcgcaaaagaatttgtctccttctctccccgggagcatcttgcagccagtgcaggaggaaaacagcaaaacttcgcctcggtcccacagaagcgacggggaattttccccccattcccattacagtgactcggatgaggcaagttagaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 11 - DNA, 6809 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccatacacttatgtatatgtgcgatgtcagtgtgtgtgcatataaagtcccaaacaagcctgtatgatattgaccaacaaggtcaaggcaaagttttgatactttcaggtcacaacctctccgcatccctctctactttgctctatctgcctgaactcctgaggacatgtttctactgcaaatggaaaatccttgtcagccagtgaggaacaaagggactatacatagatgaaaacttggctctctgctggttcctttgtttgtatgaatttatacaatttggtaaaactgccaccatgtcttacatggacagattgagtgtagattctttgaatttttgatgaagaggcgctgcactggtgatcggaattgcagtctttcctctgtaggtaacctggcttgtttccttacagtttactttctaggcctcgcctttctcacagagtgaagtcctttgttaaggttcgaatttcccataaacctgctcaataatttgtttgtgtttggcttctttgaaatactacacaaagcaatccttgtaaaaggcaaaactattccgaaggctgagaaaggagctccaggacatagattcaaagtcgctcttttcaggtagagacagctgggtaatcttatcttaactggctacatttcaaggttcccaattcaggggctttcccctctgggagcagcattctctccgggtgatgaagagctttctagtgaggagcaaaactttcagaaaaccggagggcccagagcagtctggtctgttcacaaaaattatagcaaacaaaataagcccggcggattgggtctctcctacctccagcaccaggggagatcagcacttggccccaggacagagacctgagaagtgaggtttggaagaagccaggaatccaggaaaggaggcaagattgctaaggcaccggcacagctctgagtcaaaagttgtcagtcttctttggctctggctgcggagctcaattgctcacagccctgccctttcctagggctggggcaaggaattgctacattcaggattacctgggggaaaaaccagaggcttgctttggtcccttccggtaattgaaaggactggccgtcagcgagggggaggagagagcttccctccataaatggtcccacccctgggcaaggtggctcactttggcaggtagcaaccggggagtgtgcacctgccaccagtcaagctcagccagactgtgagaagaggagaggcgaggcacaccaagggatccagtgaaccaacgacagattgaagtgcccgaacttcttcaagtgcagacagaaggaggtagggttctggaagtttctggtggtgtaggggagtcaggaagggaaaacaaggagggagagtgagtcttagttttttgctttctgtagctgttccttattttgcatatttctttctcttcaactcttttcaagtatgcctgatacgttgttctcacgaagttgacgtgaaaaacaactttcctgctggtagttaggaaacttaggagcacctcaacctgtaccttgagaacacccagagaatgctgctctttgtcgttctctataccgtgttcatatgctgcagggaaatgcaaagaatgtactgtccttatctgaccctgggagcattccatagtcaagcagcagctatcaggttgggaaagagctcctctccaaggtgtaacagaaaaggaaaatgttgatatttttcttgtttagaaagtgacagcttcatccgagaacgcggccgcgccaccatgtcccgcctgctgcatgcagaagagtgggctgaggtaaaagagttgggggaccaccatcgccatccccagccgcaccacgtcccgccgctgacgccacagccacctgctaccctgcaggcgagagaccttcccgtctacccggcagaactgtccctcctggatagcaccgacccacgcgcctggctgactcccactttgcagggcctctgcacggcacgcgccgcccagtatctgctgcattctcccgagctgggtgcctccgaggccgcggcgccccgggacgaggctgacagccagggtgagctggtaaggagaagcggctgtggcggcctcagcaagagccccgggcccgtcaaagtacgggaacagctgtgcaagctgaagggtggggttgtagtggacgagcttggctgcagccgccagcgagccccttccagcaaacaggtgaatggggtacagaagcaaaggaggctggcagcaaacgcaagggaacggcgcaggatgcacgggctgaaccacgccttcgaccagctgcgcaacgttatcccgtccttcaacaacgacaagaagctgtccaaatatgagaccctacagatggcccagatctacatcaacgctctgtcggagttgctgcagactcccaatgtcggagagcaaccgccgccgcccacagcttcctgcaaaaatgaccaccatcaccttcgcaccgcctcctcctatgaaggaggtgcgggcgcctctgcggtagctggggctcagccagccccgggagggggcccgagacctaccccgcccgggccttgccggactcgcttctcaggcccagcttcctctgggggttactcggtgcagctggacgctttgcacttcccagccttcgaggacagggccctaacagcgatgatggcacagaaggacctgtcgccttcgctgcccgggggcatcctgcagcctgtacaggaggacaacagcaaaacatctcccagatcccacagaagtgacggagagttttccccccactctcattacagtgactctgatgaggccagttagaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg